SEQUENCE LISTING (19 sequences)

SEQ ID NO: 1 - DNA, 673 bp, synthetic construct
  misc_feature 1..673: note = hCMV-LTR
  source 1..673: mol_type = other DNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaacc

SEQ ID NO: 2 - DNA, 182 bp, synthetic construct
  misc_feature 1..182: note = RU5
  source 1..182: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcag

SEQ ID NO: 3 - DNA, 151 bp, synthetic construct
  misc_feature 1..151: note = PBS
  source 1..151: mol_type = other DNA
tggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagag

SEQ ID NO: 4 - DNA, 365 bp, synthetic construct
  misc_feature 1..365: note = SL4
  source 1..365: mol_type = other DNA
atgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgat

SEQ ID NO: 5 - DNA, 241 bp, synthetic construct
  misc_feature 1..241: note = RRE
  source 1..241: mol_type = other DNA
ggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggattt

SEQ ID NO: 6 - DNA, 124 bp, synthetic construct
  misc_feature 1..124: note = PPT
  source 1..124: mol_type = other DNA
aacttttaaaagaaaaggggggattggggggtacagtgcaggggaaagaatagtagacataatagcaacagacatacaaactaaagaattacaaaaacaaattacaaaaattcaaaattttatc

SEQ ID NO: 7 - DNA, 158 bp, synthetic construct
  misc_feature 1..158: note = SYN_EN
  source 1..158: mol_type = other DNA
cgcgagttaataattaccagcgcgggccaaataaataatccgcgaggggcaggtgacgtttgcccagcgcgcgctggtaattattaacctcgcgaatattgattcgaggccgcgattgccgcaatcgcgaggggcaggtgacctttgcccagcgcgcg

SEQ ID NO: 8 - DNA, 100 bp, synthetic construct
  misc_feature 1..100: note = mTTR_EN
  source 1..100: mol_type = other DNA
cactgggaggatgttgagtaagatggaaaactactgatgacccttgcagagacagagtattaggacatgtttgaacaggggccgggcgatcagcaggtag

SEQ ID NO: 9 - DNA, 202 bp, synthetic construct
  misc_feature 1..202: note = mTTR_PRO
  source 1..202: mol_type = other DNA
gtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtc

SEQ ID NO: 10 - DNA, 23 bp, synthetic construct
  misc_feature 1..23: note = mRNA142
  source 1..23: mol_type = other DNA
tccataaagtaggaaacactaca

SEQ ID NO: 11 - DNA, 4815 bp, synthetic construct
  misc_feature 1..4815: note = coBDDFVIII6-XTEN-3aa
  source 1..4815: mol_type = other DNA
atgcagattgagctgtccacttgtttcttcctgtgcctcctgcgcttctgtttctccgccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtgtttgatgaaaacagatcctggtacctcactgaaaacatccagaggttcctcccaaaccccgcaggagtgcaactggaggaccctgagtttcaggcctcgaatatcatgcactcgattaacggttacgtgttcgactcgctgcagctgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacacatcagagagcgccacccctgaaagtggtcccgggagcgagccagccacatctgggtcggaaacgccaggcacaagtgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgagcccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtactga

SEQ ID NO: 12 - protein, 1604 aa, synthetic construct
  REGION 1..1604: note = coBDDFVIII6-XTEN-3aa
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGASSPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 13 - protein, 19 aa, synthetic construct
  REGION 1..19: note = signal
MQIELSTCFFLCLLRFCFS

SEQ ID NO: 14 - DNA, 4374 bp, synthetic construct
  misc_feature 1..4374: note = coBDDFVIII6-3aa
  source 1..4374: mol_type = other DNA
atgcagattgagctgtccacttgtttcttcctgtgcctcctgcgcttctgtttctccgccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtgtttgatgaaaacagatcctggtacctcactgaaaacatccagaggttcctcccaaaccccgcaggagtgcaactggaggaccctgagtttcaggcctcgaatatcatgcactcgattaacggttacgtgttcgactcgctgcagctgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtactga

SEQ ID NO: 15 - protein, 1457 aa, synthetic construct
  REGION 1..1457: note = coBDDFVIII-3aa
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 16 - DNA, 7769 bp, synthetic construct
  misc_feature 1..7769: note = GEC_coBDDFVIII6-XTEN-3aa
  source 1..7769: mol_type = other DNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagcacgcgagttaataattaccagcgcgggccaaataaataatccgcgaggggcaggtgacgtttgcccagcgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctggctagcgccaccatgcagattgagctgtccacttgtttcttcctgtgcctcctgcgcttctgtttctccgccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtgtttgatgaaaacagatcctggtacctcactgaaaacatccagaggttcctcccaaaccccgcaggagtgcaactggaggaccctgagtttcaggcctcgaatatcatgcactcgattaacggttacgtgttcgactcgctgcagctgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacacatcagagagcgccacccctgaaagtggtcccgggagcgagccagccacatctgggtcggaaacgccaggcacaagtgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgagcccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtactga

SEQ ID NO: 17 - protein, 1438 aa, synthetic construct
  REGION 1..1438: note = BDDFVIII
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 18 - DNA, 7053 bp, Homo sapiens
  source 1..7053: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgacggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 19 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

(19) United States
(12) Patent Application Publication
(10) Pub. No.: US 2024/0350672 A1
(43) Pub. Date: Oct. 24, 2024

(54) NUCLEIC ACIDS ENCODING FACTOR VIII POLYPEPTIDES WITH REDUCED IMMUNOGENICITY

(71) Applicant: BIOVERATIV THERAPEUTICS INC., Waltham, MA (US)

(72) Inventors: Tongyao LIU, Waltham, MA (US); Ekta Seth CHHABRA, Waltham, MA (US); Siyuan TAN, Waltham, MA (US)

(21) Appl. No.: 18/617,239

(22) Filed: Mar. 26, 2024

Related U.S. Application Data
(63) Continuation of application No. PCT/US2022/077228, filed on Sep. 29, 2022.
(60) Provisional application No. 63/250,575, filed on Sep. 30, 2021.

(51) Int. Cl. A61K 48/00; A61K 38/00; A61P 7/04; C07K 14/755; C12N 15/85

(52) U.S. Cl. CPC A61K 48/0058; A61P 7/04; C07K 14/755; C12N 15/85; A61K 38/00

(57) ABSTRACT

The present disclosure provides codon optimized Factor VIII sequences, vectors, and host cells comprising codon optimized Factor VIII sequences, polypeptides encoded by codon optimized Factor VIII sequences, and methods of producing such polypeptides.

### RELATED APPLICATIONS

This application is a continuation of International Patent Application No. PCT/US2022/077228, filed Sep. 29, 2022, which claims priority to U.S. Provisional Patent Application No. 63/250,575, filed Sep. 30, 2021, the disclosures of which are hereby incorporated by reference in their entirety.

### REFERENCE TO SEQUENCE LISTING SUBMITTED ELECTRONICALLY

The content of the electronically submitted sequence listing in xml file (Name: 752912_SA9-486PCCON_ST26.xml; Size: 56,611 bytes; Date of Creation: Mar. 26, 2024) is incorporated herein by reference in its entirety.

### BACKGROUND

A major impediment in providing a low-cost recombinant FVIII protein to patients is the high cost of commercial production. FVIII protein expresses poorly in heterologous expression systems, two to three orders of magnitude lower than similarly sized proteins. (Lynch et al., Hum. Gene. Ther.; 4:259-72 (1993). The poor expression of FVIII is due in part to the presence of cis-acting elements in the FVIII coding sequence that inhibit FVIII expression, such as transcriptional silencer elements (Hoeben et al., Blood 85:2447-2454 (1995)), matrix attachment-like sequences (MARs) (Fallux et al., Mol. Cell. Biol. 16:4264-4272 (1996)), and transcriptional elongation inhibitory elements (Koeberl et al., Hum. Gene. Ther.; 6:469-479 (1995)). Thus, there exists a need in the art for FVIII sequences that express efficiently in heterologous systems.

### SUMMARY OF THE DISCLOSURE

Disclosed are codon optimized nucleic acid molecules encoding a polypeptide with FVIII activity.

In certain aspects, disclosed herein is an isolated nucleic acid molecule comprising a nucleotide sequence having at least 85% sequence identity to SEQ ID NO:11, wherein the nucleotide sequence encodes a polypeptide with Factor VIII activity. In some embodiments, the nucleotide sequence has at least 90% sequence identity to SEQ ID NO: 11. In some embodiments, the nucleotide sequence has at least 95%, at least 96%, at least 97%, at least 98%, at least 99%, or 100% sequence identity to SEQ ID NO: 11. Also disclosed herein is an isolated nucleic acid molecule comprising the nucleotide sequence of SEQ ID NO: 11, wherein the nucleotide sequence encodes a polypeptide with Factor VIII activity.

Also disclosed herein is isolated nucleic acid molecule comprising a nucleotide sequence having at least 90%, at least 95%, at least 96%, at least 97%, at least 98%, at least 99%, or 100% sequence identity to nucleotides 58-4815 of SEQ ID NO: 11. In some embodiments, the isolated nucleic acid molecule of any one of claims 1-5, wherein the nucleotide sequence comprises nucleotides 58-4815 of SEQ ID NO: 11.

In certain aspects, disclosed herein is an isolated nucleic acid molecule comprising a nucleotide sequence having at least 85% sequence identity to SEQ ID NO: 14, wherein the nucleotide sequence encodes a polypeptide with Factor VIII activity. In some embodiments, the nucleotide sequence is at least 90% sequence identity to SEQ ID NO: 14. In some embodiments, the nucleotide sequence has at least 95%, at least 96%, at least 97%, at least 98%, at least 99%, or 100% sequence identity to SEQ ID NO: 14. Also disclosed herein is an isolated nucleic acid molecule comprising the nucleotide sequence of SEQ ID NO: 14, wherein the nucleotide sequence encodes a polypeptide with Factor VIII activity.

In another aspect, disclosed herein is an isolated nucleic acid molecule comprising a genetic cassette expressing a Factor VIII polypeptide, wherein the genetic cassette comprises a nucleotide sequence having at least 85% sequence identity to SEQ ID NO: 16. In some embodiments, the genetic cassette comprises a nucleotide sequence having at least 90% sequence identity to SEQ ID NO: 16. In some embodiments, the genetic cassette comprises a nucleotide sequence having at least 95%, at least 96%, at least 97%, at least 98%, at least 99%, or 100% sequence identity to SEQ ID NO: 16. Also disclosed herein is an isolated nucleic acid molecule comprising a genetic cassette expressing a Factor VIII polypeptide, wherein the genetic cassette comprises the nucleotide sequence of SEQ ID NO: 16.

In another aspect, disclosed herein is an isolated nucleic acid molecule comprising a genetic cassette expressing a Factor VIII polypeptide comprising: a nucleotide sequence encoding a FVIII protein comprising a nucleic acid sequence having at least 85% sequence identity to SEQ ID NO: 11 or SEQ ID NO: 14, a promoter controlling transcription of the nucleotide sequence, and a transcription termination sequence.

In some embodiments, the promoter is a liver-specific promoter. In some embodiments, the promoter is a mouse transthyretin (mTTR) promoter. In some embodiments, the promoter is a mTTR482 promoter. In some embodiments, the promoter comprises the nucleotide sequence of SEQ ID NO: 9.

In some embodiments, the isolated nucleic acid molecule further comprises an enhancer element. In some embodiments, the enhancer element is a mTTR enhancer element. In some embodiments, the mTTR enhancer element comprises the nucleotide sequence of SEQ ID NO: 8.

In some embodiments, the isolated nucleic acid molecule further comprises an synthetic enhancer sequence. In some embodiments, the synthetic enhancer sequence comprises the nucleotide sequence of SEQ ID NO: 7.

In some embodiments, the nucleic acid molecule further comprises a polypurine track (PPT). In some embodiments, the PPT sequence comprises the nucleotide sequence of SEQ ID NO: 6.

In some embodiments, the nucleic acid molecule further comprises a human CMV promoter region sequence. In some embodiments, the CMV promoter region sequence comprises the nucleotide sequence of SEQ ID NO: 1.

In some embodiments, the nucleic acid molecule further comprises a 5′ long terminal repeat (LTR) sequence. In some embodiments, the nucleic acid molecule further comprises a 3′ LTR sequence.

In some embodiments, the nucleic acid molecule further comprises a stem loop 4 sequence. In some embodiments, the stem loop 4 sequence comprises the nucleotide sequence of SEQ ID NO: 4.

In some embodiments, the nucleic acid molecule further comprises a primer binding site for SL123. In some embodiments, the primer binding site for SL123 comprises the nucleotide sequence of SEQ ID NO: 3.

In some embodiments, the nucleic acid molecule further comprises a primer binding site for RU5 region. In some embodiments, the RU5 region sequence comprises the nucleotide sequence of SEQ ID NO: 2.

In another aspect, disclosed herein is an isolated nucleic acid molecule comprising a genetic cassette expressing a Factor VIII polypeptide, wherein the genetic cassette comprises, from 5′ to 3′: a 5′ long terminal repeat (LTR) sequence, a liver-specific modified mouse transthyretin (mTTR) promoter comprising the nucleotide sequence of SEQ ID NO: 9, a nucleotide sequence encoding a FVIII protein comprising a nucleic acid sequence having at least 85% sequence identity to SEQ ID NO: 11 or SEQ ID NO: 14; and a 3′ LTR sequence.

In another aspect, disclosed herein is a vector comprising a nucleic acid molecule disclosed herein.

In another aspect, disclosed herein is a host cell comprising the nucleic acid molecule of disclosed herein. Also disclosed herein is the polypeptide produced by the host cell.

In another aspect, disclosed herein is a method of producing a polypeptide with FVIII activity, comprising: culturing the host cell disclosed herein under conditions whereby a polypeptide with FVIII activity is produced, and recovering the polypeptide with FVIII activity.

In another aspect, disclosed herein is a pharmaceutical composition comprising a nucleic acid molecule as disclosed herein. In some embodiments, the pharmaceutical composition comprises a vector comprising a nucleic acid molecule disclosed herein. In some embodiments, the pharmaceutical composition further comprises a pharmaceutically acceptable excipient.

In another aspect, disclosed herein is a kit comprising the nucleic acid molecule disclosed herein and instructions for administering the nucleic acid molecule to a subject in need thereof.

In another aspect, disclosed herein is a method of increasing expression of a polypeptide with FVIII activity in a subject comprising administering a nucleic acid molecule comprising a nucleotide sequence having at least 80% sequence identity to SEQ ID NO: 11, SEQ ID NO: 14, or SEQ ID NO: 16.

In another aspect, disclosed herein is a method of treating a bleeding disorder in a subject comprising administering a nucleic acid molecule comprising a nucleotide sequence having at least 85% sequence identity to SEQ ID NO: 11, SEQ ID NO: 14, or SEQ ID NO: 16. In some embodiments, the method of treating a bleeding disorder in a subject comprises administering the pharmaceutical composition disclosed herein. In some embodiments, the bleeding disorder is hemophilia A.

### BRIEF DESCRIPTION OF THE DRAWINGS

FIG. 1 is a graphical representation of the coBDDFVIII6-XTEN-3aa expression plasmid.

FIGS. 2A-2B are graphical representations of the peak circulating FVIII levels in neonate (2-day-old) HemA mice administered lentivirus expressing coBDDFVIII6-XTEN-3aa at 1.5×10⁹, 3.0×10⁹, 6.0×10⁹ or 1.3×10¹⁰ TU/kg dose via temporal vein injection, as measured by FVIII plasma activity (FIG. 2A) and FVIII plasma antigen levels (FIG. 2B) for approximately 25 weeks.

FIG. 3 is a graphical representation of the peak circulating FVIII levels in adult (16-week-old) HemA mice administered lentivirus expressing coBDDFVIII6-XTEN-3aa at 1.3×10¹⁰ or 3.7×10¹⁰ TU/kg dose via tail vein injection, as measured by FVIII plasma activity for approximately 25 weeks.

FIGS. 4A-4B are graphical representations of peak plasma levels of human FVIII activity (FIG. 4A) and human FVIII antigen levels (FIG. 4B) in male pigtail macaques administered 3×10⁹ TU/kg or 6×10⁹ TU/kg lentivirus expressing coBDDFVIII6-XTEN-3aa. FVIII plasma activity (FIG. 4A) and FVIII plasma antigen levels (FIG. 4B) are presented as averages across multiple timepoints.

### DETAILED DESCRIPTION

The present disclosure is directed to codon optimized nucleic acid molecules encoding polypeptides with Factor VIII (FVIII) activity, vectors, and host cells comprising optimized nucleic acid molecules, polypeptides encoded by optimized nucleic acid molecules, and methods of producing such polypeptides. The present disclosure is also directed to methods of treating bleeding disorders such as hemophilia comprising administering to the subject an optimized FVIII nucleic acid sequence, a vector comprising the optimized nucleic acid sequence, or the polypeptide encoded thereby.

The present disclosure meets an important need in the art by providing optimized FVIII sequences that demonstrate increased expression in host cells, improved yield of FVIII protein in methods to produce recombinant FVIII, and potentially result in greater therapeutic efficacy when used in gene therapy methods. In certain embodiments, the disclosure describes an isolated nucleic acid molecule comprising a nucleotide sequence which has sequence homology to the nucleotide sequence of SEQ ID NO: 11. In certain embodiments, the disclosure describes an isolated nucleic acid molecule comprising a nucleotide sequence which has sequence homology to the nucleotide sequence of SEQ ID NO: 14. In certain embodiments, the disclosure describes an isolated nucleic acid molecule comprising a nucleotide sequence which has sequence homology to the nucleotide sequence of SEQ ID NO: 16.

In order to provide a clear understanding of the specification and claims, the following definitions are provided below.

### Definitions

It is to be noted that the term “a” or “an” entity refers to one or more of that entity: for example, “a nucleotide sequence” is understood to represent one or more nucleotide sequences. As such, the terms “a” (or “an”), “one or more,” and “at least one” can be used interchangeably herein.

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

The term “isolated” for the purposes of the present disclosure designates a biological material (cell, polypeptide, polynucleotide, or a fragment, variant, or derivative thereof) that has been removed from its original environment (the environment in which it is naturally present). For example, a polynucleotide present in the natural state in a plant or an animal is not isolated, however the same polynucleotide separated from the adjacent nucleic acids in which it is naturally present, is considered “isolated.” No particular level of purification is required. Recombinantly produced polypeptides and proteins expressed in host cells are considered isolated for the purpose of the disclosure, as are native or recombinant polypeptides which have been separated, fractionated, or partially or substantially purified by any suitable technique.

“Nucleic acids,” “nucleic acid molecules,” “oligonucleotide,” and “polynucleotide” are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein.

As used herein, a “coding region” or “coding sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide. Two or more coding regions can be present in a single polynucleotide construct, e.g., on a single vector, or in separate polynucleotide constructs, e.g., on separate (different) vectors. It follows, then, that a single vector can contain just a single coding region or comprise two or more coding regions.

Certain proteins secreted by mammalian cells are associated with a secretory signal peptide which is cleaved from the mature protein once export of the growing protein chain across the rough endoplasmic reticulum has been initiated. Those of ordinary skill in the art are aware that signal peptides are generally fused to the N-terminus of the polypeptide and are cleaved from the complete or “full-length” polypeptide to produce a secreted or “mature” form of the polypeptide. In certain embodiments, a native signal peptide or a functional derivative of that sequence that retains the ability to direct the secretion of the polypeptide that is operably associated with it. Alternatively, a heterologous mammalian signal peptide, e.g., a human tissue plasminogen activator (TPA) or mouse β-glucuronidase signal peptide, or a functional derivative thereof, can be used.

The term “downstream” refers to a nucleotide sequence that is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence. In certain embodiments, upstream nucleotide sequences relate to sequences that are located on the 5′ side of a coding region or starting point of transcription. For example, most promoters are located upstream of the start site of transcription.

As used herein, the terms “genetic cassette”, “expression cassette”, and “genetic expression cassette” are used interchangeably and refer to a DNA sequence capable of directing expression of a particular polynucleotide sequence in an appropriate host cell, comprising a promoter operably linked to a polynucleotide sequence of interest. A genetic cassette may encompass nucleotide sequences located upstream (5′ non-coding sequences), within, or downstream (3′ non-coding sequences) of a coding region, and which influence the transcription, RNA processing, stability, or translation of the associated coding region. If a coding region is intended for expression in a eukaryotic cell, a polyadenylation signal and transcription termination sequence will usually be located 3′ to the coding sequence. In some embodiments, the genetic cassette comprises a polynucleotide which encodes a gene product. In some embodiments, the genetic cassette comprises a polynucleotide which encodes a miRNA. In some embodiments, the genetic cassette comprises a heterologous polynucleotide sequence. A polynucleotide which encodes a product, e.g., a miRNA or a gene product (e.g., a polypeptide such as a therapeutic protein), can include a promoter and/or other expression (e.g., transcription or translation) control sequences operably associated with one or more coding regions. In an operable association a coding region for a gene product, e.g., a polypeptide, is associated with one or more regulatory regions in such a way as to place expression of the gene product under the influence or control of the regulatory region(s). For example, a coding region and a promoter are “operably associated” if induction of promoter function results in the transcription of mRNA encoding the gene product encoded by the coding region, and if the nature of the linkage between the promoter and the coding region does not interfere with the ability of the promoter to direct the expression of the gene product or interfere with the ability of the DNA template to be transcribed. Other expression control sequences, besides a promoter, for example enhancers, operators, repressors, and transcription termination signals, can also be operably associated with a coding region to direct gene product expression.

“Expression control sequences” refer to regulatory nucleotide sequences, such as promoters, enhancers, terminators, and the like, that provide for the expression of a coding sequence in a host cell. Expression control sequences generally encompass any regulatory nucleotide sequence which facilitates the efficient transcription and translation of the coding nucleic acid to which it is operably linked. Non-limiting examples of expression control sequences include promoters, enhancers, translation leader sequences, introns, polyadenylation recognition sequences, RNA processing sites, effector binding sites, or stem-loop structures. A variety of expression control sequences are known to those skilled in the art. These include, without limitation, expression control sequences which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other expression control sequences include those derived from vertebrate genes such as actin, heat shock protein, bovine growth hormone and rabbit β-globin, as well as other sequences capable of controlling gene expression in eukaryotic cells. Additional suitable expression control sequences include tissue-specific promoters and enhancers as well as lymphokine-inducible promoters (e.g., promoters inducible by interferons or interleukins). Other expression control sequences include intronic sequences, post-transcriptional regulatory elements, and polyadenylation signals. Additional exemplary expression control sequences are discussed elsewhere in the present disclosure.

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES).

The term “expression” as used herein refers to a process by which a polynucleotide produces a gene product, for example, an RNA or a polypeptide. It includes without limitation transcription of the polynucleotide into messenger RNA (mRNA), transfer RNA (tRNA), small hairpin RNA (shRNA), small interfering RNA (siRNA) or any other RNA product, and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage. The term “yield,” as used herein, refers to the amount of a polypeptide produced by the expression of a gene.

A “vector” refers to any vehicle for the cloning of and/or transfer of a nucleic acid into a host cell. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “vector” includes both viral and nonviral vehicles for introducing the nucleic acid into a cell in vitro, ex vivo or in vivo. A large number of vectors are known and used in the art including, for example, plasmids, modified eukaryotic viruses, or modified bacterial viruses. Insertion of a polynucleotide into a suitable vector can be accomplished by ligating the appropriate polynucleotide fragments into a chosen vector that has complementary cohesive termini.

Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), β-galactosidase (LacZ), β-glucuronidase (Gus), and the like. Selectable markers can also be considered to be reporters.

The term “selectable marker” refers to an identifying factor, usually an antibiotic or chemical resistance gene, that is able to be selected for based upon the marker gene's effect, i.e., resistance to an antibiotic, resistance to a herbicide, colorimetric markers, enzymes, fluorescent markers, and the like, wherein the effect is used to track the inheritance of a nucleic acid of interest and/or to identify a cell or organism that has inherited the nucleic acid of interest. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like.

The term “reporter gene” refers to a nucleic acid encoding an identifying factor that is able to be identified based upon the reporter gene's effect, wherein the effect is used to track the inheritance of a nucleic acid of interest, to identify a cell or organism that has inherited the nucleic acid of interest, and/or to measure gene expression induction or transcription. Examples of reporter genes known and used in the art include: luciferase (Luc), green fluorescent protein (GFP), chloramphenicol acetyltransferase (CAT), β-galactosidase (LacZ), β-glucuronidase (Gus), and the like. Selectable marker genes can also be considered reporter genes.

“Promoter” and “promoter sequence” are used interchangeably and refer to a DNA sequence capable of controlling the expression of a coding sequence or functional RNA. In general, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity. Additional exemplary promoters are discussed elsewhere in the present disclosure.

The promoter sequence is typically bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

The term “plasmid” refers to an extra-chromosomal element often carrying a gene that is not part of the central metabolism of the cell, and usually in the form of circular double-stranded DNA molecules. Such elements can be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

Eukaryotic viral vectors that can be used include, but are not limited to, adenovirus vectors, retrovirus vectors, adeno-associated virus vectors, poxvirus, e.g., vaccinia virus vectors, baculovirus vectors, or herpesvirus vectors. Non-viral vectors include plasmids, liposomes, electrically charged lipids (cytofectins), DNA-protein complexes, and biopolymers.

A “cloning vector” refers to a “replicon,” which is a unit length of a nucleic acid that replicates sequentially and which comprises an origin of replication, such as a plasmid, phage or cosmid, to which another nucleic acid segment can be attached so as to bring about the replication of the attached segment. Certain cloning vectors are capable of replication in one cell type, e.g., bacteria and expression in another, e.g., eukaryotic cells. Cloning vectors typically comprise one or more sequences that can be used for selection of cells comprising the vector and/or one or more multiple cloning sites for insertion of nucleic acid sequences of interest.

The term “expression vector” refers to a vehicle designed to enable the expression of an inserted nucleic acid sequence following insertion into a host cell. The inserted nucleic acid sequence is placed in operable association with regulatory regions as described above.

Vectors are introduced into host cells by methods well known in the art, e.g., transfection, electroporation, microinjection, transduction, cell fusion, DEAE dextran, calcium phosphate precipitation, lipofection (lysosome fusion), use of a gene gun, or a DNA vector transporter.

“Culture,” “to culture” and “culturing,” as used herein, means to incubate cells under in vitro conditions that allow for cell growth or division or to maintain cells in a living state. “Cultured cells,” as used herein, means cells that are propagated in vitro.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acctylation, phosphorylation, amidation, derivatization by known protecting/blocking groups, proteolytic cleavage, or modification by non-naturally occurring amino acids. A polypeptide can be derived from a natural biological source or produced recombinant technology, but is not necessarily translated from a designated nucleic acid sequence. It can be generated in any manner, including by chemical synthesis.

The term “amino acid” includes alanine (Ala or A); arginine (Arg or R); asparaginc (Asn or N); aspartic acid (Asp or D); cysteine (Cys or C); glutamine (Gln or Q); glutamic acid (Glu or E); glycine (Gly or G); histidine (His or H); isoleucine (Ile or I): leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr or Y); and valine (Val or V). Non-traditional amino acids are also within the scope of the disclosure and include norleucine, omithine, norvaline, homoserine, and other amino acid residue analogues such as those described in Ellman et al. Meth. Enzym. 202:301-336 (1991). To generate such non-naturally occurring amino acid residues, the procedures of Noren et al. Science 244:182 (1989) and Ellman et al., supra, can be used. Briefly, these procedures involve chemically activating a suppressor tRNA with a non-naturally occurring amino acid residue followed by in vitro transcription and translation of the RNA. Introduction of the non-traditional amino acid can also be achieved using peptide chemistries known in the art. As used herein, the term “polar amino acid” includes amino acids that have net zero charge, but have non-zero partial charges in different portions of their side chains (e.g., M, F, W, S, Y, N, Q, C). These amino acids can participate in hydrophobic interactions and electrostatic interactions. As used herein, the term “charged amino acid” includes amino acids that can have non-zero net charge on their side chains (e.g., R, K, H, E, D). These amino acids can participate in hydrophobic interactions and electrostatic interactions.

Also included in the present disclosure are fragments or variants of polypeptides, and any combination thereof. The term “fragment” or “variant” when referring to polypeptide binding domains or binding molecules of the present disclosure include any polypeptides which retain at least some of the properties (e.g., FcRn binding affinity for an FcRn binding domain or Fc variant, coagulation activity for an FVIII variant, or FVIII binding activity for the VWF fragment) of the reference polypeptide. Fragments of polypeptides include proteolytic fragments, as well as deletion fragments, in addition to specific antibody fragments discussed elsewhere herein, but do not include the naturally occurring full-length polypeptide (or mature polypeptide). Variants of polypeptide binding domains or binding molecules of the present disclosure include fragments as described above, and also polypeptides with altered amino acid sequences due to amino acid substitutions, deletions, or insertions. Variants can be naturally or non-naturally occurring. Non-naturally occurring variants can be produced using art-known mutagenesis techniques. Variant polypeptides can comprise conservative or non-conservative amino acid substitutions, deletions or additions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another embodiment, a string of amino acids can be conservatively replaced with a structurally similar string that differs in order and/or composition of side chain family members.

The term “percent identity” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case can be, as determined by the match between strings of such sequences. “Identity” can be readily calculated by known methods, including but not limited to those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991). Preferred methods to determine identity are designed to give the best match between the sequences tested. Methods to determine identity are codified in publicly available computer programs. Sequence alignments and percent identity calculations can be performed using sequence analysis software such as the Megalign program of the LASERGENE bioinformatics computing suite (DNASTAR Inc., Madison, WI), the GCG suite of programs (Wisconsin Package Version 9.0, Genetics Computer Group (GCG), Madison, WI), BLASTP, BLASTN, BLASTX (Altschul et al., J. Mol. Biol. 215:403 (1990)), and DNASTAR (DNASTAR, Inc. 1228 S. Park St. Madison, WI 53715 USA). Within the context of this application, it will be understood that where sequence analysis software is used for analysis, that the results of the analysis will be based on the “default values” of the program referenced, unless otherwise specified. As used herein “default values” will mean any set of values or parameters which originally load with the software when first initialized. For the purposes of determining percent identity between an optimized BDD FVIII sequence of the disclosure and a reference sequence, only nucleotides in the reference sequence corresponding to nucleotides in the optimized BDD FVIII sequence of the disclosure are used to calculate percent identity. For example, when comparing a full length FVIII nucleotide sequence containing the B domain to an optimized B domain deleted (BDD) FVIII nucleotide sequence of the disclosure, the portion of the alignment including the A1, A2, A3, C1, and C2 domain will be used to calculate percent identity. The nucleotides in the portion of the full length FVIII sequence encoding the B domain (which will result in a large “gap” in the alignment) will not be counted as a mismatch. In addition, in determining percent identity between an optimized BDD FVIII sequence of the disclosure, or a designated portion thereof (e.g., nucleotides 2183-4474 and 4924-7006 of SEQ ID NO:16), and a reference sequence, percent identity will be calculated by aligning dividing the number of matched nucleotides by the total number of nucleotides in the complete sequence of the optimized BDD-FVIII sequence, or a designated portion thereof, as recited herein.

As used herein, the term “insertion site” refers to a position in a FVIII polypeptide, or fragment, variant, or derivative thereof, which is immediately upstream of the position at which a heterologous moiety can be inserted. An “insertion site” is specified as a number, the number corresponding to the number of the amino acid in mature native FVIII (SEQ ID NO: 18) to which the insertion site corresponds, which is immediately N-terminal to the position of the insertion. For example, the phrase “a3 comprises a heterologous moiety at an insertion site which corresponds to amino acid 1656 of SEQ ID NO: 24” indicates that the heterologous moiety is located between two amino acids corresponding to amino acid 1656 and amino acid 1657 of SEQ ID NO: 24.

The phrase “immediately downstream of an amino acid” as used herein refers to position right next to the terminal carboxyl group of the amino acid. Similarly, the phrase “immediately upstream of an amino acid” refers to the position right next to the terminal amine group of the amino acid.

The terms “inserted,” “is inserted,” “inserted into” or grammatically related terms, as used herein refers to the position of a heterologous moiety in a recombinant FVIII polypeptide, relative to the analogous position in native mature human FVIII (SEQ ID NO: 18).

As used herein, the term “half-life” refers to a biological half-life of a particular polypeptide in vivo. Half-life can be represented by the time required for half the quantity administered to a subject to be cleared from the circulation and/or other tissues in the animal. When a clearance curve of a given polypeptide is constructed as a function of time, the curve is usually biphasic with a rapid α-phase and longer β-phase. The α-phase typically represents an equilibration of the administered Fc polypeptide between the intra- and extra-vascular space and is, in part, determined by the size of the polypeptide. The β-phase typically represents the catabolism of the polypeptide in the intravascular space. In some embodiments, FVIII and chimeric proteins comprising FVIII are monophasic, and thus do not have an alpha phase, but just the single beta phase. Therefore, in certain embodiments, the term half-life as used herein refers to the half-life of the polypeptide in the β-phase.

The term “linked” as used herein refers to a first amino acid sequence or nucleotide sequence covalently or non-covalently joined to a second amino acid sequence or nucleotide sequence, respectively. The first amino acid or nucleotide sequence can be directly joined or juxtaposed to the second amino acid or nucleotide sequence or alternatively an intervening sequence can covalently join the first sequence to the second sequence. The term “linked” means not only a fusion of a first amino acid sequence to a second amino acid sequence at the C-terminus or the N-terminus, but also includes insertion of the whole first amino acid sequence (or the second amino acid sequence) into any two amino acids in the second amino acid sequence (or the first amino acid sequence, respectively). In one embodiment, the first amino acid sequence can be linked to a second amino acid sequence by a peptide bond or a linker. The first nucleotide sequence can be linked to a second nucleotide sequence by a phosphodiester bond or a linker. The linker can be a peptide or a polypeptide (for polypeptide chains) or a nucleotide or a nucleotide chain (for nucleotide chains) or any chemical moiety (for both polypeptide and polynucleotide chains). The term “linked” is also indicated by a hyphen (-).

As used herein the term “associated with” refers to a covalent or non-covalent bond formed between a first amino acid chain and a second amino acid chain. In one embodiment, the term “associated with” means a covalent, non-peptide bond or a non-covalent bond. This association can be indicated by a colon, i.e., (:). In another embodiment, it means a covalent bond except a peptide bond. For example, the amino acid cysteine comprises a thiol group that can form a disulfide bond or bridge with a thiol group on a second cysteine residue. In most naturally occurring IgG molecules, the CHI and CL regions are associated by a disulfide bond and the two heavy chains are associated by two disulfide bonds at positions corresponding to 239 and 242 using the Kabat numbering system (position 226 or 229, EU numbering system). Examples of covalent bonds include, but are not limited to, a peptide bond, a metal bond, a hydrogen bond, a disulfide bond, a sigma bond, a pi bond, a delta bond, a glycosidic bond, an agnostic bond, a bent bond, a dipolar bond, a Pi backbond, a double bond, a triple bond, a quadruple bond, a quintuple bond, a sextuple bond, conjugation, hyperconjugation, aromaticity, hapticity, or antibonding. Non-limiting examples of non-covalent bond include an ionic bond (e.g., cation-pi bond or salt bond), a metal bond, a hydrogen bond (e.g., dihydrogen bond, dihydrogen complex, low-barrier hydrogen bond, or symmetric hydrogen bond), van der Walls force, London dispersion force, a mechanical bond, a halogen bond, aurophilicity, intercalation, stacking, entropic force, or chemical polarity.

“Hemostasis,” as used herein, means the stopping or slowing of bleeding or hemorrhage; or the stopping or slowing of blood flow through a blood vessel or body part.

“Hemostatic disorder,” as used herein, means a genetically inherited or acquired condition characterized by a tendency to hemorrhage, either spontaneously or as a result of trauma, due to an impaired ability or inability to form a fibrin clot. Examples of such disorders include the hemophilias. The three main forms are hemophilia A (factor VIII deficiency), hemophilia B (factor IX deficiency or “Christmas disease”) and hemophilia C (factor XI deficiency, mild bleeding tendency). Other hemostatic disorders include, e.g., von Willebrand disease, Factor XI deficiency (PTA deficiency), Factor XII deficiency, deficiencies or structural abnormalities in fibrinogen, prothrombin, Factor V, Factor VII, Factor X or factor XIII, Bernard-Soulier syndrome, which is a defect or deficiency in GPIb. GPIb, the receptor for VWF, can be defective and lead to lack of primary clot formation (primary hemostasis) and increased bleeding tendency), and thrombasthenia of Glanzman and Naegeli (Glanzmann thrombasthenia). In liver failure (acute and chronic forms), there is insufficient production of coagulation factors by the liver; this can increase bleeding risk.

The isolated nucleic acid molecules, isolated polypeptides, or vectors comprising the isolated nucleic acid molecule of the disclosure can be used prophylactically. As used herein the term “prophylactic treatment” refers to the administration of a molecule prior to a bleeding episode. In one embodiment, the subject in need of a general hemostatic agent is undergoing, or is about to undergo, surgery. A polynucleotide, polypeptide, or vector of the disclosure can be administered prior to or after surgery as a prophylactic. The polynucleotide, polypeptide, or vector of the disclosure can be administered during or after surgery to control an acute bleeding episode. The surgery can include, but is not limited to, liver transplantation, liver resection, dental procedures, or stem cell transplantation.

The isolated nucleic acid molecules, isolated polypeptides, or vectors of the disclosure are also used for on-demand treatment. The term “on-demand treatment” refers to the administration of an isolated nucleic acid molecule, isolated polypeptide, or vector in response to symptoms of a bleeding episode or before an activity that can cause bleeding. In one aspect, the on-demand treatment can be given to a subject when bleeding starts, such as after an injury, or when bleeding is expected, such as before surgery. In another aspect, the on-demand treatment can be given prior to activities that increase the risk of bleeding, such as contact sports.

As used herein the term “acute bleeding” refers to a bleeding episode regardless of the underlying cause. For example, a subject can have trauma, uremia, a hereditary bleeding disorder (e.g., factor VII deficiency) a platelet disorder, or resistance owing to the development of antibodies to clotting factors.

“Treat,” “treatment,” “treating,” as used herein refers to, e.g., the reduction in severity of a disease or condition; the reduction in the duration of a disease course; the amelioration of one or more symptoms associated with a disease or condition; the provision of beneficial effects to a subject with a disease or condition, without necessarily curing the disease or condition, or the prophylaxis of one or more symptoms associated with a disease or condition. In one embodiment, the term “treating” or “treatment” means maintaining a FVIII trough level at least about 1 IU/dL, 2 IU/dL, 3 IU/dL, 4 IU/dL, 5 IU/dL, 6 IU/dL, 7 IU/dL, 8 IU/dL, 9 IU/dL, 10 IU/dL, 11 IU/dL, 12 IU/dL, 13 IU/dL, 14 IU/dL, 15 IU/dL, 16 IU/dL, 17 IU/dL, 18 IU/dL, 19 IU/dL, or 20 IU/dL in a subject by administering an isolated nucleic acid molecule, isolated polypeptide or vector of the disclosure. In another embodiment, treating or treatment means maintaining a FVIII trough level between about 1 and about 20 IU/dL, about 2 and about 20 IU/dL, about 3 and about 20 IU/dL, about 4 and about 20 IU/dL, about 5 and about 20 IU/dL, about 6 and about 20 IU/dL, about 7 and about 20 IU/dL, about 8 and about 20 IU/dL, about 9 and about 20 IU/dL, or about 10 and about 20 IU/dL. Treatment or treating of a disease or condition can also include maintaining FVIII activity in a subject at a level comparable to at least about 1%, 2%, 3%, 4%, 5%, 6%, 7%, 8%, 9%, 10%, 11%, 12%, 13%, 14%, 15%, 16%, 17%, 18%, 19%, or 20% of the FVIII activity in a non-hemophiliac subject. The minimum trough level required for treatment can be measured by one or more known methods and can be adjusted (increased or decreased) for each person.

“Administering,” as used herein, means to give a pharmaceutically acceptable Factor VIII-encoding nucleic acid molecule, Factor VIII polypeptide, or vector comprising a Factor VIII-encoding nucleic acid molecule of the disclosure to a subject via a pharmaceutically acceptable route. Routes of administration can be intravenous, e.g., intravenous injection and intravenous infusion. Additional routes of administration include, e.g., subcutaneous, intraneural, intraocular, intrathecal, intramuscular, oral, nasal, and pulmonary administration. The nucleic acid molecules, polypeptides, and vectors can be administered as part of a pharmaceutical composition comprising at least one excipient.

As used herein, the phrase “subject in need thereof” includes subjects, such as mammalian subjects, that would benefit from administration of a nucleic acid molecule, a polypeptide, or vector of the disclosure, e.g., to improve hemostasis. In one embodiment, the subjects include, but are not limited to, individuals with hemophilia. In another embodiment, the subjects include, but are not limited to, the individuals who have developed a FVIII inhibitor and thus are in need of a bypass therapy. The subject can be an adult or a minor (e.g., under 12 years old).

As used herein, the term “clotting factor,” refers to molecules, or analogs thereof, naturally occurring or recombinantly produced which prevent or decrease the duration of a bleeding episode in a subject. In other words, it means molecules having pro-clotting activity, i.e., are responsible for the conversion of fibrinogen into a mesh of insoluble fibrin causing the blood to coagulate or clot. An “activatable clotting factor” is a clotting factor in an inactive form (e.g., in its zymogen form) that is capable of being converted to an active form.

“Clotting activity,” as used herein, means the ability to participate in a cascade of biochemical reactions that culminates in the formation of a fibrin clot and/or reduces the severity, duration or frequency of hemorrhage or bleeding episode.

As used herein the terms “heterologous” or “exogenous” refer to such molecules that are not normally found in a given context, e.g., in a cell or in a polypeptide. For example, an exogenous or heterologous molecule can be introduced into a cell and are only present after manipulation of the cell, e.g., by transfection or other forms of genetic engineering or a heterologous amino acid sequence can be present in a protein in which it is not naturally found.

As used herein, the term “heterologous nucleotide sequence” refers to a nucleotide sequence that does not naturally occur with a given polynucleotide sequence. In one embodiment, the heterologous nucleotide sequence encodes a polypeptide capable of extending the half-life of FVIII. In another embodiment, the heterologous nucleotide sequence encodes a polypeptide that increases the hydrodynamic radius of FVIII. In other embodiments, the heterologous nucleotide sequence encodes a polypeptide that improves one or more pharmacokinetic properties of FVIII without significantly affecting its biological activity or function (e.g., its procoagulant activity). In some embodiments, FVIII is linked or connected to the polypeptide encoded by the heterologous nucleotide sequence by a linker.

A “reference nucleotide sequence,” when used herein as a comparison to a nucleotide sequence of the disclosure, is a polynucleotide sequence essentially identical to the nucleotide sequence of the disclosure except that the portions corresponding to FVIII sequence are not optimized.

As used herein, the term “optimized,” with regard to nucleotide sequences, refers to a polynucleotide sequence that encodes a polypeptide, wherein the polynucleotide sequence has been mutated to enhance a property of that polynucleotide sequence. In some embodiments, the optimization is done to increase transcription levels, increase translation levels, increase steady-state mRNA levels, increase or decrease the binding of regulatory proteins such as general transcription factors, increase or decrease splicing, or increase the yield of the polypeptide produced by the polynucleotide sequence. Examples of changes that can be made to a polynucleotide sequence to optimize it include codon optimization, G/C content optimization, removal of repeat sequences, removal of AT rich elements, removal of cryptic splice sites, removal of cis-acting elements that repress transcription or translation, adding or removing poly-T or poly-A sequences, adding sequences around the transcription start site that enhance transcription, such as Kozak consensus sequences, removal of sequences that could form stem loop structures, removal of destabilizing sequences, removal of CpG motifs, and two or more combinations thereof.

### Polynucleotide Sequences

Certain aspects of the present disclosure are directed to a nucleic acid molecule comprising a genetic cassette, e.g., encoding a therapeutic protein and/or a miRNA. In some embodiments, the genetic cassette encodes a therapeutic protein. In some embodiments, the therapeutic protein comprises a clotting factor. In some embodiments, the genetic cassette encodes a miRNA. In some embodiments, the nucleic acid molecule further comprises at least one noncoding region. In certain embodiments, the at least one non-coding region comprises a promoter sequence, an intron, an expression control sequence, or any combination thereof.

In some embodiments, the genetic cassette comprises a nucleotide sequence encoding a FVIII polypeptide, wherein the nucleotide sequence is codon optimized. In some embodiments, the genetic cassette comprises a nucleotide sequence encoding a codon optimized FVIII driven by a mTTR promoter. In some embodiments, the genetic cassette comprises a nucleotide sequence which is disclosed in International Application No. PCT/US2017/015879, which is incorporated by reference in its entirety. In some embodiments, the genetic cassette is a “hFVIIIco6XTEN” genetic cassette as described in PCT/US2017/015879. In some embodiments, the reference nucleotide sequence corresponds to a hFVIIIco6XTEN sequence, as disclosed in PCT/US2017/015879.

In some embodiments, the genetic cassette comprises codon optimized cDNA encoding a B-domain deleted (BDD) codon-optimized human Factor VIII molecule. In some embodiments, the genetic cassette comprises a nucleotide sequence having at least 50%, at least 55%, at least 60%, at least 65%, at least 70%, at least 75%, at least 80%, at least 85%, at least 90%, at least 95%, at least 96%, at least 97%, at least 98%, at least 99%, or 100% sequence identity to SEQ ID NO: 14. In some embodiments, the genetic cassette comprises a nucleotide sequence encoding a coBDDFVIII6-3aa polypeptide.

In some embodiments, the genetic cassette further comprises a nucleotide sequence encoding an XTEN polypeptide. In some embodiments, the genetic cassette comprises a codon optimized cDNA encoding B-domain deleted (BDD) codon-optimized human Factor VIII (BDDcoFVIII) fused with a 144 amino acid XTEN polypeptide. In some embodiments, the genetic cassette comprises the nucleotide sequence set forth as SEQ ID NO: 11. In some embodiments, the genetic cassette comprises a nucleotide sequence having at least 50%, at least 55%, at least 60%, at least 65%, at least 70%, at least 75%, at least 80%, at least 85%, at least 90%, at least 95%, at least 96%, at least 97%, at least 98%, at least 99%, or 100% sequence identity to SEQ ID NO: 11. In some embodiments, the genetic cassette comprises a nucleotide sequence encoding a coBDDFVIII6-XTEN-3aa polypeptide.

In some embodiments, the genetic cassette comprises the nucleotide sequence set forth as SEQ ID NO: 16. In some embodiments, the genetic cassette comprises a nucleotide sequence having at least 50%, at least 55%, at least 60%, at least 65%, at least 70%, at least 75%, at least 80%, at least 85%, at least 90%, at least 95%, at least 96%, at least 97%, at least 98%, at least 99%, or 100% sequence identity to SEQ ID NO: 16.

In some embodiments, the present disclosure is directed to codon optimized nucleic acid molecules encoding a polypeptide with FVIII activity. In some embodiments, the polynucleotide encodes a full-length FVIII polypeptide. In other embodiments, the nucleic acid molecule encodes a B domain-deleted (BDD) FVIII polypeptide, wherein all or a portion of the B domain of FVIII is deleted. In one particular embodiment, the nucleic acid molecule encodes a polypeptide comprising an amino acid sequence having at least about 80%, at least about 85%, at least about 86%, at least about 87%, at least about 88%, at least about 89%, at least about 90%, at least about 95%, at least about 96%, at least about 97%, at least about 98%, or at least about 99% sequence identity to SEQ ID NO: 12 or a fragment thereof.

In some embodiments, the nucleic acid molecule of the disclosure encodes a FVIII polypeptide comprising a signal peptide or a fragment thereof. In other embodiments, the nucleic acid molecule encodes a FVIII polypeptide which lacks a signal peptide. In some embodiments, the signal peptide comprises the amino acid sequence of SEQ ID NO: 13.

In one embodiment, the genetic cassette is a single stranded nucleic acid. In another embodiment, the genetic cassette is a double stranded nucleic acid. In another embodiment, the genetic cassette is a closed-end double stranded nucleic acid (ceDNA).

“A polypeptide with FVIII activity” as used herein means a functional FVIII polypeptide in its normal role in coagulation, unless otherwise specified. The term a polypeptide with FVIII activity includes a functional fragment, variant, analog, or derivative thereof that retains the function of full-length wild-type Factor VIII in the coagulation pathway. “A polypeptide with FVIII activity” is used interchangeably with FVIII protein, FVIII polypeptide, or FVIII. Examples of FVIII functions include, but are not limited to, an ability to activate coagulation, an ability to act as a cofactor for factor IX, or an ability to form a tenase complex with factor IX in the presence of Ca²⁺ and phospholipids, which then converts Factor X to the activated form Xa. In one embodiment, a polypeptide having FVIII activity comprises two polypeptide chains, the first chain having the FVIII heavy chain and the second chain having the FVIII light chain. In another embodiment, the polypeptide having FVIII activity is single chain FVIII. Single chain FVIII can contain one or more mutation or substitutions at amino acid residue 1645 and/or 1648 corresponding to mature human FVIII sequence (SEQ ID NO: 19). See International Application No. PCT/US2012/045784, incorporated herein by reference in its entirety. The FVIII protein can be the human, porcine, canine, rat, or murine FVIII protein. In addition, comparisons between FVIII from humans and other species have identified conserved residues that are likely to be required for function. See, e.g., Cameron et al. (1998) Thromb. Haemost. 79:317-22; and U.S. Pat. No. 6,251,632.

A number of tests are available to assess the FVIII activity of a polypeptide: activated partial thromboplastin time (aPTT) test, chromogenic assay, ROTEM® assay, prothrombin time (PT) test (also used to determine INR), fibrinogen testing (often by the Clauss method), platelet count, platelet function testing (often by PFA-100), TCT, bleeding time, mixing test (whether an abnormality corrects if the patient's plasma is mixed with normal plasma), coagulation factor assays, antiphosholipid antibodies, D-dimer, genetic tests (e.g., factor V Leiden, prothrombin mutation G20210A), dilute Russell's viper venom time (dRVVT), miscellaneous platelet function tests, thromboclastography (TEG or Sonoclot), thromboelastometry (TEM®, e.g, ROTEM®), or euglobulin lysis time (ELT).

The aPTT test is a performance indicator measuring the efficacy of both the “intrinsic” (also referred to the contact activation pathway) and the common coagulation pathways. This test is commonly used to measure clotting activity of commercially available recombinant clotting factors, e.g., FVIII or FIX. It is used in conjunction with prothrombin time (PT), which measures the extrinsic pathway.

ROTEM® analysis provides information on the whole kinetics of haemostasis: clotting time, clot formation, clot stability and lysis. The different parameters in thromboelastometry are dependent on the activity of the plasmatic coagulation system, platelet function, fibrinolysis, or many factors which influence these interactions. This assay can provide a complete view of secondary haemostasis.

The “B domain” of FVIII, as used herein, is the same as the B domain known in the art that is defined by internal amino acid sequence identity and sites of proteolytic cleavage by thrombin, e.g., residues Ser741-Arg1648 of full length human FVIII (SEQ ID NO: 20). The other human FVIII domains are defined by the following amino acid residues: A1, residues Ala1-Arg372; A2, residues Ser373-Arg740; A3, residues Ser1690-Ile2032; C1, residues Arg2033-Asn2172; C2, residues Ser2173-Tyr2332. The A3-C1-C2 sequence includes residues Ser1690-Tyr2332. The remaining sequence, residues Glu1649-Arg1689, is usually referred to as the FVIII light chain activation peptide. The locations of the boundaries for all of the domains, including the B domains, for porcine, mouse and canine FVIII are also known in the art. An example of a BDD FVIII is REFACTOR recombinant BDD FVIII (Wyeth Pharmaceuticals, Inc.).

A “B domain deleted FVIII” can have the full or partial deletions disclosed in U.S. Pat. Nos. 6,316,226, 6,346,513, 7,041,635, 5,789,203, 6,060,447, 5,595,886, 6,228,620, 5,972,885, 6,048,720, 5,543,502, 5,610,278, 5,171,844, 5,112,950, 4,868,112, and 6,458,563, each of which is incorporated herein by reference in its entirety. Other examples of B domain deleted FVIII are disclosed in Hoeben R. C., et al. (1990) J. Biol. Chem. 265 (13): 7318-7323; Meulien et al. (1988), Protein Eng. 2 (4): 301-6; Toole et al. (1986) Proc. Natl. Acad. Sci. U.S.A. 83, 5939-5942; Eaton, et al. (1986) Biochemistry 25:8343-8347; (Sarver, et al. (1987) DNA 6:553-564; European Patent No. 295597; and International Publication Nos. WO 91/09122, WO 88/00831, and WO 87/04187, each of which is incorporated herein by reference in its entirety. Each of the foregoing deletions can be made in any FVIII sequence.

### Codon Optimization

In one embodiment, the present disclosure provides an isolated nucleic acid molecule comprising a nucleotide sequence that encodes a polypeptide with FVIII activity, wherein the nucleic acid sequence has been codon optimized. In some embodiments, the sequence that encodes a polypeptide with FVIII activity is codon optimized for human expression. In other embodiments, the sequence that encodes a polypeptide with FVIII activity is codon optimized for murine expression.

The term “codon-optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA. Such optimization includes replacing at least one, or more than one, or a significant number, of codons with one or more codons that are more frequently used in the genes of that organism.

Deviations in the nucleotide sequence that comprises the codons encoding the amino acids of any polypeptide chain allow for variations in the sequence coding for the gene. Since each codon consists of three nucleotides, and the nucleotides comprising DNA are restricted to four specific bases, there are 64 possible combinations of nucleotides, 61 of which encode amino acids (the remaining three codons encode signals ending translation). As a result, many amino acids are designated by more than one codon. For example, the amino acids alanine and proline are coded for by four triplets, serine and arginine by six, whereas tryptophan and methionine are coded by just one triplet. This degeneracy allows for DNA base composition to vary over a wide range without altering the amino acid sequence of the proteins encoded by the DNA.

Many organisms display a bias for use of particular codons to code for insertion of a particular amino acid in a growing peptide chain. Codon preference, or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

Given the large number of gene sequences available for a wide variety of animal, plant and microbial species, the relative frequencies of codon usage have been calculated. Codon usage tables are available, for example, at the “Codon Usage Database” available at www.kazusa.or.jp/codon/(visited Jun. 18, 2012). See Nakamura, Y., et al. Nucl. Acids Res. 28:292 (2000).

Randomly assigning codons at an optimized frequency to encode a given polypeptide sequence can be done manually by calculating codon frequencies for each amino acid, and then assigning the codons to the polypeptide sequence randomly. Additionally, various algorithms and computer software programs can be used to calculate an optimal sequence.

Codon optimization may also include removal of potential immunogenic sequences from the protein sequence encoded by a nucleotide sequence. In some embodiments, in silico methods can be used to identify potential immunogenic sequences in the protein or nucleotide sequence. Non-limiting examples of these methods include identification of human leukocyte antigen (HLA) alleles (e.g. DR, DP, DQ), and identification of major histocompatibility complex class II (MHCII) binding sites in a given protein sequence. In some embodiments, public databases such as the Immune Epitope Database and Analysis Resource (IEDB) (http://www.icdb.org/) can be used to identify potential immunogenic sequences (see, e.g. Zhang Q et al. Nucleic Acids Res (2008) 36: W513-8; Kim Y et al. Nucleic Acids Res (2012) 40: W525-30; Dhanda et al. Nucleic Acids Res (2019) 47: W502-W506). In some embodiments, the NetMHCIIpan 3.0 method can be used to identify potential immunogenic sequences, as described in Lamberth K, et al. Sci Transl Med. 2017; 9 (372): eaag1286. In some embodiments, the nucleotide sequence encoding potential immunogenic sequences is deleted.

### Heterologous Nucleotide Sequences

In some embodiments, the isolated nucleic acid molecules of the disclosure further comprise a heterologous nucleotide sequence. In some embodiments, the isolated nucleic acid molecules of the disclosure further comprise at least one heterologous nucleotide sequence. The heterologous nucleotide sequence can be linked with the optimized BDD-FVIII nucleotide sequences of the disclosure at the 5′ end, at the 3′ end, or inserted into the middle of the optimized BDD-FVIII nucleotide sequence. Thus, in some embodiments, the heterologous amino acid sequence encoded by the heterologous nucleotide sequence is linked to the N-terminus or the C-terminus of the FVIII amino acid sequence encoded by the nucleotide sequence or inserted between two amino acids in the FVIII amino acid sequence. In some embodiments, the heterologous amino acid sequence can be inserted between two amino acids at one or more insertion site. In some embodiments, the heterologous amino acid sequence can be inserted within the FVIII polypeptide encoded by the nucleic acid molecule of the disclosure at any site disclosed in International Publication No. WO 2013/123457 A1, WO 2015/106052 A1 or U.S. Publication No. 2015/0158929 A1, each of which are incorporated by reference in their entirety.

In some embodiments, the heterologous amino acid sequence encoded by the heterologous nucleotide sequence is inserted within the B domain or a fragment thereof. In some embodiments, the heterologous amino acid sequence is inserted within the FVIII immediately downstream of an amino acid corresponding to amino acid 745 of wild type mature human FVIII (SEQ ID NO: 19). In one particular embodiment, the FVIII comprises a deletion of amino acids 746-1637, corresponding to wild type mature human FVIII (SEQ ID NO: 19), and the heterologous amino acid sequence encoded by the heterologous nucleotide sequence is inserted immediately downstream of amino acid 745, corresponding to wild type mature human FVIII (SEQ ID NO: 19). The insertion sites of FVIII referenced herein indicate the amino acid position corresponding to the amino acid position of wild type mature human FVIII (SEQ ID NO: 19).

In some embodiments, the heterologous moiety is a peptide or a polypeptide with either unstructured or structured characteristics that are associated with the prolongation of in vivo half-life when incorporated in a protein of the disclosure. Non-limiting examples include albumin, albumin fragments, Fc fragments of immunoglobulins, the C-terminal peptide (CTP) of the β subunit of human chorionic gonadotropin, a HAP sequence, an XTEN sequence, a transferrin or a fragment thereof, a PAS polypeptide, polyglycine linkers, polyserine linkers, albumin-binding moieties, or any fragments, derivatives, variants, or combinations of these polypeptides.

In certain embodiments, a heterologous moiety improves one or more pharmacokinetic properties of the FVIII protein without significantly affecting its biological activity or function. In some embodiments, a heterologous moiety increases the in vivo and/or in vitro half-life of the FVIII protein of the disclosure. In vivo half-life of a FVIII protein can be determined by any methods known to those of skill in the art, e.g., activity assays (chromogenic assay or one stage clotting aPTT assay), ELISA, ROTEM™, etc.

In other embodiments, a heterologous moiety increases stability of the FVIII protein of the disclosure or a fragment thereof (e.g., a fragment comprising a heterologous moiety after proteolytic cleavage of the FVIII protein). As used herein, the term “stability” refers to an art-recognized measure of the maintenance of one or more physical properties of the FVIII protein in response to an environmental condition (e.g., an elevated or lowered temperature). In certain aspects, the physical property can be the maintenance of the covalent structure of the FVIII protein (e.g., the absence of proteolytic cleavage, unwanted oxidation or deamidation). In other aspects, the physical property can also be the presence of the FVIII protein in a properly folded state (e.g., the absence of soluble or insoluble aggregates or precipitates). In one aspect, the stability of the FVIII protein is measured by assaying a biophysical property of the FVIII protein, for example thermal stability, pH unfolding profile, stable removal of glycosylation, solubility, biochemical function (e.g., ability to bind to a protein, receptor or ligand), etc., and/or combinations thereof. In another aspect, biochemical function is demonstrated by the binding affinity of the interaction. In one aspect, a measure of protein stability is thermal stability, i.e., resistance to thermal challenge. Stability can be measured using methods known in the art, such as, HPLC (high performance liquid chromatography), SEC (size exclusion chromatography), DLS (dynamic light scattering), etc. Methods to measure thermal stability include, but are not limited to differential scanning calorimetry (DSC), differential scanning fluorimetry (DSF), circular dichroism (CD), and thermal challenge assay.

In some embodiments, a heterologous moiety comprises one or more XTEN sequences, fragments, variants, or derivatives thereof. As used here “XTEN sequence” refers to extended length polypeptides with non-naturally occurring, substantially non-repetitive sequences that are composed mainly of small hydrophilic amino acids, with the sequence having a low degree or no secondary or tertiary structure under physiologic conditions. As a heterologous moiety, XTENs can serve as a half-life extension moiety. In addition, XTEN can provide desirable properties including but are not limited to enhanced pharmacokinetic parameters and solubility characteristics. Other advantageous properties which may be conferred by introducing an XTEN sequence include enhanced conformational flexibility, enhanced aqueous solubility, high degree of protease resistance, low immunogenicity, low binding to mammalian receptors, or increased hydrodynamic (or Stokes) radii.

XTEN can have varying lengths for insertion into or linkage to FVIII. In some embodiments, the XTEN sequence useful for the disclosure is a peptide or a polypeptide having greater than about 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, or 2000 amino acid residues. In certain embodiments, XTEN is a peptide or a polypeptide having greater than about 20 to about 3000 amino acid residues, greater than 30 to about 2500 residues, greater than 40 to about 2000 residues, greater than 50 to about 1500 residues, greater than 60 to about 1000 residues, greater than 70 to about 900 residues, greater than 80 to about 800 residues, greater than 90 to about 700 residues, greater than 100 to about 600 residues, greater than 110 to about 500 residues, or greater than 120 to about 400 residues. In one particular embodiment, the XTEN comprises an amino acid sequence of longer than 42 amino acids and shorter than 144 amino acids in length.

The XTEN sequence of the disclosure can comprise one or more sequence motif of 5 to 14 (e.g., 9 to 14) amino acid residues or an amino acid sequence at least 80%, 90%, 91%, 92%, 93%, 94%, 95%, 96%, 97%, 98%, or 99% identical to the sequence motif, wherein the motif comprises, consists essentially of, or consists of 4 to 6 types of amino acids (e.g., 5 amino acids) selected from the group consisting of glycine (G), alanine (A), serine(S), threonine (T), glutamate (E) and proline (P). See US 2010-0239554 A1.

Examples of XTEN sequences that can be used as heterologous moieties in chimeric proteins of the disclosure are disclosed, e.g., in U.S. Patent Publication Nos. 2010/0239554 A1, 2010/0323956 A1, 2011/0046060 A1, 2011/0046061 A1, 2011/0077199 A1, or 2011/0172146 A1, or International Patent Publication Nos. WO 2010091122 A1, WO 2010144502 A2, WO 2010144508 A1, WO 2011028228 A1, WO 2011028229 A1, or WO 2011028344 A2, each of which is incorporated by reference herein in its entirety.

The one or more XTEN sequences can be inserted at the C-terminus or at the N-terminus of the amino acid sequence encoded by the nucleotide sequence or inserted between two amino acids in the amino acid sequence encoded by the nucleotide sequence. For example, the XTEN can be inserted between two amino acids at one or more insertion sites. Examples of sites within FVIII that are permissible for XTEN insertion can be found in, e.g., International Publication No. WO 2013/123457 A1 or U.S. Publication No. 2015/0158929 A1, which are herein incorporated by reference in their entirety.

In certain embodiments, the heterologous moiety is a peptide linker.

As used herein, the terms “peptide linkers” or “linker moieties” refer to a peptide or polypeptide sequence (e.g., a synthetic peptide or polypeptide sequence) which connects two domains in a linear amino acid sequence of a polypeptide chain.

In some embodiments, heterologous nucleotide sequences encoding peptide linkers can be inserted between the optimized FVIII polynucleotide sequences of the disclosure and a heterologous nucleotide sequence encoding, for example, one of the heterologous moieties described above, such as albumin. Peptide linkers can provide flexibility to the chimeric polypeptide molecule. Linkers are not typically cleaved, however such cleavage can be desirable. In one embodiment, these linkers are not removed during processing.

A type of linker which can be present in a chimeric protein of the disclosure is a protease cleavable linker which comprises a cleavage site (i.e., a protease cleavage site substrate, e.g., a factor XIa, Xa, or thrombin cleavage site) and which can include additional linkers on either the N-terminal of C-terminal or both sides of the cleavage site. These cleavable linkers when incorporated into a construct of the disclosure result in a chimeric molecule having a heterologous cleavage site.

In one embodiment, an FVIII polypeptide encoded by a nucleic acid molecule of the instant disclosure comprises two or more Fc domains or moieties linked via a cscFc linker to form an Fc region comprised in a single polypeptide chain. The cscFc linker is flanked by at least one intracellular processing site, i.e., a site cleaved by an intracellular enzyme. Cleavage of the polypeptide at the at least one intracellular processing site results in a polypeptide which comprises at least two polypeptide chains.

Other peptide linkers can optionally be used in a construct of the disclosure, e.g., to connect an FVIII protein to an Fc region. Some exemplary linkers that can be used in connection with the disclosure include, e.g., polypeptides comprising GlySer amino acids described in more detail below.

In one embodiment, the peptide linker is synthetic, i.e., non-naturally occurring. In one embodiment, a peptide linker includes peptides (or polypeptides) (which can or cannot be naturally occurring) which comprise an amino acid sequence that links or genetically fuses a first linear sequence of amino acids to a second linear sequence of amino acids to which it is not naturally linked or genetically fused in nature. For example, in one embodiment the peptide linker can comprise non-naturally occurring polypeptides which are modified forms of naturally occurring polypeptides (e.g., comprising a mutation such as an addition, substitution or deletion). In another embodiment, the peptide linker can comprise non-naturally occurring amino acids. In another embodiment, the peptide linker can comprise naturally occurring amino acids occurring in a linear sequence that does not occur in nature. In still another embodiment, the peptide linker can comprise a naturally occurring polypeptide sequence.

In another embodiment, a peptide linker comprises or consists of a gly-ser linker. As used herein, the term “gly-ser linker” refers to a peptide that consists of glycine and serine residues. In certain embodiments, said gly-ser linker can be inserted between two other sequences of the peptide linker. In other embodiments, a gly-ser linker is attached at one or both ends of another sequence of the peptide linker. In yet other embodiments, two or more gly-ser linker are incorporated in series in a peptide linker. In one embodiment, a peptide linker of the disclosure comprises at least a portion of an upper hinge region (e.g., derived from an IgG1, IgG2, IgG3, or IgG4 molecule), at least a portion of a middle hinge region (e.g., derived from an IgG1, IgG2, IgG3, or IgG4 molecule) and a series of gly/ser amino acid residues.

Peptide linkers of the disclosure are at least one amino acid in length and can be of varying lengths. In one embodiment, a peptide linker of the disclosure is from about 1 to about 50 amino acids in length. As used in this context, the term “about” indicates +/−two amino acid residues. Since linker length must be a positive integer, the length of from about 1 to about 50 amino acids in length, means a length of from 1-3 to 48-52 amino acids in length. In another embodiment, a peptide linker of the disclosure is from about 10 to about 20 amino acids in length. In another embodiment, a peptide linker of the disclosure is from about 15 to about 50 amino acids in length. In another embodiment, a peptide linker of the disclosure is from about 20 to about 45 amino acids in length. In another embodiment, a peptide linker of the disclosure is from about 15 to about 35 or about 20 to about 30 amino acids in length. In another embodiment, a peptide linker of the disclosure is from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 500, 1000, or 2000 amino acids in length. In one embodiment, a peptide linker of the disclosure is 20 or 30 amino acids in length.

In some embodiments, the peptide linker can comprise at least two, at least three, at least four, at least five, at least 10, at least 20, at least 30, at least 40, at least 50, at least 60, at least 70, at least 80, at least 90, or at least 100 amino acids. In other embodiments, the peptide linker can comprise at least 200, at least 300, at least 400, at least 500, at least 600, at least 700, at least 800, at least 900, or at least 1,000 amino acids. In some embodiments, the peptide linker can comprise at least about 10, 20, 30, 40, 50, 60, 70, 80, 90, 100, 150, 200, 300, 400, 500, 600, 700, 800, 900, 1000, 1100, 1200, 1300, 1400, 1500, 1600, 1700, 1800, 1900, or 2000 amino acids. The peptide linker can comprise 1-5 amino acids, 1-10 amino acids, 1-20 amino acids, 10-50 amino acids, 50-100 amino acids, 100-200 amino acids, 200-300 amino acids, 300-400 amino acids, 400-500 amino acids, 500-600 amino acids, 600-700 amino acids, 700-800 amino acids, 800-900 amino acids, or 900-1000 amino acids.

Peptide linkers can be introduced into polypeptide sequences using techniques known in the art. Modifications can be confirmed by DNA sequence analysis. Plasmid DNA can be used to transform host cells for stable production of the polypeptides produced.

### Expression Control Sequences

In some embodiments, the nucleic acid molecule or vector of the disclosure further comprises at least one expression control sequence. For example, the isolated nucleic acid molecule of the disclosure can be operably linked to at least one expression control sequence. The expression control sequence can, for example, be a promoter sequence or promoter-enhancer combination.

Constitutive mammalian promoters include, but are not limited to, the promoters for the following genes: hypoxanthine phosphoribosyl transferase (HPRT), adenosine deaminase, pyruvate kinase, beta-actin promoter, and other constitutive promoters. Exemplary viral promoters which function constitutively in eukaryotic cells include, for example, promoters from the cytomegalovirus (CMV), simian virus (e.g., SV40), papilloma virus, adenovirus, human immunodeficiency virus (HIV), Rous sarcoma virus, cytomegalovirus, the long terminal repeats (LTR) of Moloney leukemia virus, and other retroviruses, and the thymidine kinase promoter of herpes simplex virus. Other constitutive promoters are known to those of ordinary skill in the art. The promoters useful as gene expression sequences of the disclosure also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

In one embodiment, the disclosure includes expression of a transgene under the control of a tissue specific promoter and/or enhancer. In another embodiment, the promoter or other expression control sequence selectively enhances expression of the transgene in liver cells. In certain embodiments, the promoter or other expression control sequence selectively enhances expression of the transgene in hepatocytes, sinusoidal cells, and/or endothelial cells. In one particular embodiment, the promoter or other expression control sequence selective enhances expression of the transgene in endothelial cells. In certain embodiments, the promoter or other expression control sequence selective enhances expression of the transgene in muscle cells, the central nervous system, the eye, the liver, the heart, or any combination thereof. Examples of liver specific promoters include, but are not limited to, a mouse transthyretin promoter (mTTR), a native human factor VIII promoter, human alpha-1-antitrypsin promoter (hAAT), human albumin minimal promoter, and mouse albumin promoter. In some embodiments, the nucleic acid molecules disclosed herein comprise a mTTR promoter. The mTTR promoter is described in Costa et al. (1986) Mol. Cell. Biol. 6:4697. The FVIII promoter is described in Figueiredo and Brownlee, 1995, J. Biol. Chem. 270:11828-11838. In some embodiments, the promoter is selected from a liver specific promoter (e.g., al-antitrypsin (AAT)), a muscle specific promoter (e.g., muscle creatine kinase (MCK), myosin heavy chain alpha (aMHC), myoglobin (MB), and desmin (DES)), a synthetic promoter (e.g., SPc5-12, 2R5Sc5-12, dMCK, and tMCK), or any combination thereof.

In some embodiments, the transgene expression is targeted to the liver. In certain embodiments, the transgene expression is targeted to hepatocytes. In other embodiment, the transgene expression is targeted to endothelial cells. In one particular embodiment, the transgene expression is targeted to any tissue that naturally expressed endogenous FVIII. In some embodiments, the transgene expression is targeted to the central nervous system. In certain embodiments, the transgene expression is targeted to neurons. In some embodiments, the transgene expression is targeted to afferent neurons. In some embodiments, the transgene expression is targeted to efferent neurons. In some embodiments, the transgene expression is targeted to interneurons. In some embodiments, the transgene expression is targeted to glial cells. In some embodiments, the transgene expression is targeted to astrocytes. In some embodiments, the transgene expression is targeted to oligodendrocytes. In some embodiments, the transgene expression is targeted to microglia. In some embodiments, the transgene expression is targeted to ependymal cells. In some embodiments, the transgene expression is targeted to Schwann cells. In some embodiments, the transgene expression is targeted to satellite cells. In some embodiments, the transgene expression is targeted to muscle tissue. In some embodiments, the transgene expression is targeted to smooth muscle. In some embodiments, the transgene expression is targeted to cardiac muscle. In some embodiments, the transgene expression is targeted to skeletal muscle. In some embodiments, the transgene expression is targeted to the eye. In some embodiments, the transgene expression is targeted to a photoreceptor cell. In some embodiments, the transgene expression is targeted to retinal ganglion cell.

Other promoters useful in the nucleic acid molecules disclosed herein include a mouse transthyretin promoter (mTTR), a native human factor VIII promoter, a human alpha-1-antitrypsin promoter (hAAT), a human albumin minimal promoter, a mouse albumin promoter, a tristetraprolin (TTP; also known as ZFP36) promoter, a CASI promoter, a CAG promoter, a cytomegalovirus (CMV) promoter, an al-antitrypsin (AAT) promoter, a muscle creatine kinase (MCK) promoter, myosin heavy chain alpha (aMHC) promoter, a myoglobin (MB) promoter, desmin (DES) promoter, a SPc5-12 promoter, a 2R5Sc5-12 promoter, a dMCK promoter, and a tMCK promoter, a phosphoglycerate kinase (PGK) promoter, or any combinations thereof.

In some embodiments, the nucleic acid molecules disclosed herein comprise a transthyretin (TTR) promoter. In some embodiments, the promoter is a mouse transthyretin (mTTR) promoter. Non-limiting examples of mTTR promoters include the mTTR202 promoter, mTTR202opt promoter, and mTTR482 promoter, as disclosed in U.S. Publication No. US2019/0048362, which is incorporated by reference herein in its entirety. In some embodiments, the promoter is a liver-specific modified mouse transthyretin (mTTR) promoter. In some embodiments, the promoter is the liver-specific modified mouse transthyretin (mTTR) promoter mTTR482. Examples of mTTR482 promoters are described in Kyostio-Moore et al. (2016) Mol Ther Methods Clin Dev. 3:16006, and Nambiar B. et al. (2017) Hum Gene Ther Methods, 28 (1): 23-28. In some embodiments, the promoter is a liver-specific modified mouse transthyretin (mTTR) promoter comprising the nucleic acid sequence of SEQ ID NO: 9.

Expression levels can be further enhanced to achieve therapeutic efficacy using one or more enhancer elements. One or more enhancers can be provided either alone or together with one or more promoter elements. Typically, the expression control sequence comprises a plurality of enhancer elements and a tissue specific promoter. In one embodiment, an enhancer comprises one or more copies of the α-1-microglobulin/bikunin enhancer (Rouet et al. (1992) J. Biol. Chem. 267:20765-20773; Rouet et al. (1995), Nucleic Acids Res. 23:395-404; Rouet et al (1998) Biochem. J. 334:577-584; Ill et al. (1997) Blood Coagulation Fibrinolysis 8: S23-S30). In some embodiments, the enhancer is derived from liver specific transcription factor binding sites, such as EBP, DBP, HNF1, HNF3, HNF4, HNF6, with Enh1, comprising HNF1, (sense)-HNF3, (sense)-HNF4, (antisense)-HNF1, (antisense)-HNF6, (sense)-EBP, (antisense)-HNF4 (antisense). In some embodiments, the enhancer is the mTTR482 enhancer comprising the nucleic acid sequence of SEQ ID NO: 8.

In some embodiments, the enhancer comprises one or two modified prothrombin enhancers (pPrT2), one or two alpha 1-microbikunin enhancers (A1MB2), a modified mouse albumin enhancer (mEalb), a hepatitis B virus enhancer II (HE11), or a CRM8 enhancer. In some embodiments, the enhancer is a synthetic enhancer. In some embodiments, the enhancer is a synthetic enhancer comprising the nucleic acid sequence of SEQ ID NO: 7.

In some embodiments, the nucleic acid molecules disclosed herein comprise an intron or intronic sequence. In some embodiments, the intronic sequence is a naturally occurring intronic sequence. In some embodiments, the intronic sequence is a synthetic sequence. In some embodiments, the intronic sequence is derived from a naturally occurring intronic sequence. In some embodiments, the intronic sequence is a hybrid synthetic intron or chimeric intron. In some embodiments, the intronic sequence is a chimeric intron that consists of chicken beta-actin/rabbit beta-globin intron and has been modified to eliminate five existing ATG sequences to reduce false translation starts. In certain embodiments, the intronic sequence comprises the SV40 small T intron.

In some embodiments, the nucleic acid molecule disclosed herein comprises one or more DNA nuclear targeting sequences (DTSs). A DTS promotes translocation of DNA molecules containing such sequences into the nucleus. In certain embodiments, the DTS comprises an SV40 enhancer sequence. In certain embodiments, the DTS comprises a c-Myc enhancer sequence. In some embodiments, the nucleic acid molecule comprises DTSs that are located between the first ITR and the second ITR. In some embodiments, the nucleic acid molecule comprises a DTS located 3′ to the first ITR and 5′ to the transgene (e.g. FVIII protein). In some embodiments, the nucleic acid molecule comprises a DTS located 3′ to the transgene and 5′ to the second ITR on the nucleic acid molecule.

In some embodiments, the nucleic acid molecule disclosed herein comprises a toll-like receptor 9 (TLR9) inhibition sequence. Exemplary TLR9 inhibition sequences are described in, e.g., Trieu et al. (2006) Crit Rev Immunol. 26 (6): 527-44; Ashman et al. Int'l Immunology 23 (3): 203-14.

### Vector Systems

Some embodiments of the present disclosure are directed to vectors comprising one or more codon optimized nucleic acid molecules encoding a polypeptide with FVIII activity described herein, host cells comprising the vectors, and methods of treating a bleeding disorder using the vectors. The present disclosure meets an important need in the art by providing a vector comprising an optimized FVIII sequence that demonstrates increased expression in a subject and potentially result in greater therapeutic efficacy when used in gene therapy methods.

Suitable vectors for the disclosure include expression vectors, viral vectors, and plasmid vectors. In one embodiment, the vector is a viral vector.

As used herein, an expression vector refers to any nucleic acid construct which contains the necessary elements for the transcription and translation of an inserted coding sequence, or in the case of an RNA viral vector, the necessary elements for replication and translation, when introduced into an appropriate host cell. Expression vectors can include plasmids, phagemids, viruses, and derivatives thereof.

Expression vectors of the disclosure will include optimized polynucleotides encoding the BDD FVIII protein described herein. In one embodiment, the optimized coding sequences for the BDD FVIII protein is operably linked to an expression control sequence. As used herein, two nucleic acid sequences are operably linked when they are covalently linked in such a way as to permit each component nucleic acid sequence to retain its functionality. A coding sequence and a gene expression control sequence are said to be operably linked when they are covalently linked in such a way as to place the expression or transcription and/or translation of the coding sequence under the influence or control of the gene expression control sequence. Two DNA sequences are said to be operably linked if induction of a promoter in the 5′ gene expression sequence results in the transcription of the coding sequence and if the nature of the linkage between the two DNA sequences does not (1) result in the introduction of a frame-shift mutation, (2) interfere with the ability of the promoter region to direct the transcription of the coding sequence, or (3) interfere with the ability of the corresponding RNA transcript to be translated into a protein. Thus, a gene expression sequence would be operably linked to a coding nucleic acid sequence if the gene expression sequence were capable of effecting transcription of that coding nucleic acid sequence such that the resulting transcript is translated into the desired protein or polypeptide.

Viral vectors include, but are not limited to, nucleic acid sequences from the following viruses: retrovirus, such as Moloney murine leukemia virus, Harvey murine sarcoma virus, murine mammary tumor virus, and Rous sarcoma virus; lentivirus; adenovirus; adeno-associated virus; SV40-type viruses; polyomaviruses; Epstein-Barr viruses; papilloma viruses; herpes virus; vaccinia virus; polio virus; and RNA virus such as a retrovirus. One can readily employ other vectors well-known in the art. Certain viral vectors are based on non-cytopathic eukaryotic viruses in which non-essential genes have been replaced with the gene of interest. In one embodiment, the virus is an adeno-associated virus, a double-stranded DNA virus. The adeno-associated virus can be engineered to be replication-deficient and is capable of infecting a wide range of cell types and species.

One or more of different AAV vector sequences derived from nearly any serotype can be used in accord with the present disclosure. Choice of a particular AAV vector sequence will be guided by known parameters such as tropism of interest, required vector yields, etc. Generally, the AAV serotypes have genomic sequences of significant homology at the amino acid and the nucleic acid levels, provide a related set of genetic functions, produce virions which are related, and replicate and assemble similarly. For the genomic sequence of the various AAV serotypes and an overview of the genomic similarities see, e.g., GenBank Accession number U89790; GenBank Accession number J01901; GenBank Accession number AF043303; GenBank Accession number AF085716; Chlorini et al. (1997) J. Vir. 71:6823-33; Srivastava et al. (1983) J. Vir. 45:555-64; Chlorini et al. (1999) J. Vir. 73:1309-1319; Rutledge et al. (1998), J. Vir. 72:309-319; or Wu et al. (2000) J. Vir. 74:8635-47. AAV serotypes 1, 2, 3, 4 and 5 are an illustrative source of AAV nucleotide sequences for use in the context of the present disclosure. AAV6, AAV7, AAV8 or AAV9 or newly developed AAV-like particles obtained by e.g. capsid shuffling techniques and AAV capsid libraries, or from newly designed, developed or evolved ITR's are also suitable for certain disclosure applications. See Dalkara et al. (2013), Sci. Transl. Med. 5 (189): 189ra76; Kotterman M A (2014) Nat. Rev. Genet. 15 (7): 455.

Other vectors include plasmid vectors. Plasmid vectors have been extensively described in the art and are well-known to those of skill in the art. See, e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operably encoded within the plasmid. Some commonly used plasmids available from commercial suppliers include pBR322, pUC18, pUC19, various pcDNA plasmids, pRC/CMV, various pCMV plasmids, pSV40, and pBlueScript. Additional examples of specific plasmids include pcDNA3.1, catalog number V79020; pcDNA3.1/hygro, catalog number V87020; pcDNA4/myc-His, catalog number V86320; and pBudCE4.1, catalog number V53220, all from Invitrogen (Carlsbad, CA.). Other plasmids are well-known to those of ordinary skill in the art. Additionally, plasmids can be custom designed using standard molecular biology techniques to remove and/or add specific fragments of DNA.

In certain embodiments, it will be useful to include within the vector one or more miRNA target sequences which, for example, are operably linked to the optimized FVIII transgene. More than one copy of a miRNA target sequence included in the vector can increase the effectiveness of the system. For example, vectors which express more than one transgene can have the transgene under control of more than one miRNA target sequence, which can be the same or different. The miRNA target sequences can be in tandem, but other arrangements are also included. The transgene genetic cassette, containing miRNA target sequences, can also be inserted within the vector in antisense orientation. Examples of the miRNA target sequences are described at WO2007/000668, WO2004/094642, WO2010/055413, or WO2010/125471, which are incorporated herein by reference in their entireties. However in certain other embodiments, the vector will not include any miRNA target sequence. Choice of whether or not to include an miRNA target sequence (and how many) will be guided by known parameters such as the intended tissue target, the level of expression required, etc.

### Lentiviral Vectors

Lentiviruses include members of the bovine lentivirus group, equine lentivirus group, feline lentivirus group, ovinecaprine lentivirus group, and primate lentivirus group. The development of lentivirus vectors for gene therapy has been reviewed in Klimatcheva et al. (1999) Frontiers in Bioscience 4:481-496. The design and use of lentiviral vectors suitable for gene therapy is described for example in U.S. Pat. Nos. 6,207,455 and 6,615,782. Examples of lentivirus include, but are not limited to, HIV-1, HIV-2, HIV-1/HIV-2 pseudotype, HIV-1/SIV, FIV, caprine arthritis encephalitis virus (CAEV), equine infectious anemia virus, and bovine immunodeficiency virus.

In some embodiments, the lentiviral vector of the present disclosure is “third-generation” lentiviral vector. As used herein, the term “third-generation” lentiviral vector refers to a lentiviral packaging system that has the characteristics of a second-generation vector system, and that further lacks a functional tat gene, such as one from which the tat gene has been deleted or inactivated. Typically, the gene encoding rev is provided on a separate expression construct. Sec, e.g., Dull et al. (1998) J. Virol. 72:8463-8471. As used herein, a “second-generation” lentiviral vector system refers to a lentiviral packaging system that lacks functional accessory genes, such as one from which the accessory genes vif, vpr, vpu, and nef have been deleted or inactivated. Sec, e.g., Zufferey et al. (1997) Nat. Biotechnol. 15:871-875. As used herein, “packaging system” refers to a set of viral constructs comprising genes that encode viral proteins involved in packaging a recombinant virus. Typically, the constructs of the packaging system will ultimately be incorporated into a packaging cell.

In some embodiments, the third-generation lentiviral vector of the present disclosure is a self-inactivating lentiviral vector. In some embodiments, the lentiviral vector is a VSV.G pseudo type lentiviral vector. In some embodiments, the lentiviral vector comprises a hepatocyte-specific promoter for transgene expression. In some embodiments, the hepatocyte-specific promoter is an enhanced transthyretin promoter. In some embodiments, the lentiviral vector comprises one or more target sequences for miR-142 to reduce immune response to the transgene product. In some embodiments, incorporating one or more target sequences for miR-142 into a lentiviral vector of the present disclosure allows for a desired transgene expression profile. For example, incorporating one or more target sequences for miR-142 may suppress transgene expression in intravascular and extravascular hematopoietic lineages, whereas transgene expression is maintained in nonhematopoietic cells. No oncogenesis has been detected in tumor prone mice treated with the lentivirus vector system of the present disclosure. See Brown et al. (2007) Blood 110:4144-52, Brown at al. (2006) Nat. Ned. 12:585-91, and Cantore et al. (2015) Sci. Transl. Med. 7 (277): 277ra28.

Lentiviral vectors of the disclosure include codon optimized polynucleotides encoding the BDD FVIII protein described herein. In one embodiment, the optimized coding sequences for the BDD FVIII protein is operably linked to an expression control sequence. As used herein, two nucleic acid sequences are operably linked when they are covalently linked in such a way as to permit each component nucleic acid sequence to retain its functionality. A coding sequence and a gene expression control sequence are said to be operably linked when they are covalently linked in such a way as to place the expression or transcription and/or translation of the coding sequence under the influence or control of the gene expression control sequence. Two DNA sequences are said to be operably linked if induction of a promoter in the 5′ gene expression sequence results in the transcription of the coding sequence and if the nature of the linkage between the two DNA sequences does not (1) result in the introduction of a frame-shift mutation, (2) interfere with the ability of the promoter region to direct the transcription of the coding sequence, or (3) interfere with the ability of the corresponding RNA transcript to be translated into a protein. Thus, a gene expression sequence would be operably linked to a coding nucleic acid sequence if the gene expression sequence were capable of effecting transcription of that coding nucleic acid sequence such that the resulting transcript is translated into the desired protein or polypeptide.

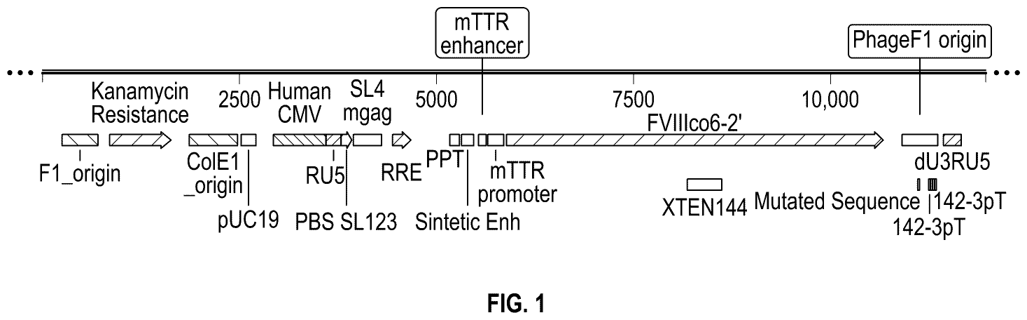

A schematic representation of an exemplary lentiviral vector embodiment disclosed herein is presented as FIG. 1. Additional information on the generation of exemplary lentiviral vector embodiments can be found in Example 2. Further discussion of the design of retroviral vectors for gene therapy is provided in Poletti & Mavilio, Viruses. 2021 (13): 1526.

In certain embodiments, the lentiviral vector is a vector of a recombinant lentivirus capable of infecting non-dividing cells. In certain embodiments, the lentiviral vector is a vector of a recombinant lentivirus capable of infecting liver cells (e.g., hepatocytes). The lentiviral genome and the proviral DNA typically have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The κ′ and 3′ LTR's serve to promote transcription and polyadenylation of the virion RNA's. The LTR contains all other cis-acting sequences necessary for viral replication. Lentiviruses have additional genes including vif, vpr, tat, rev, vpu, nef and vpx (in HIV-1, HIV-2 and/or SIV).

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA.

In some embodiments, the lentiviral vector comprises the primer binding site (PBS) for stem loop 123 (SL123). In some embodiments, the PBS comprises the nucleotide sequence of SEQ ID NO: 3. In some embodiments, the lentiviral vector comprises a Psi stem-loop 4 (SL4) sequence. In some embodiments, the lentiviral vector comprises the nucleotide sequence of SEQ ID NO: 4. Further discussion of Psi and related sequences can be found in Kim et al. PLOS ONE 2012.7 (11): c50148.

However, the resulting mutant remains capable of directing the synthesis of all virion proteins. The disclosure provides a method of producing a recombinant lentivirus capable of infecting a non-dividing cell comprising transfecting a suitable host cell with two or more vectors carrying the packaging functions, namely gag, pol and env, as well as rev and tat. As will be disclosed herein below, vectors lacking a functional tat gene are desirable for certain applications. Thus, for example, a first vector can provide a nucleic acid encoding a viral gag and a viral pol and another vector can provide a nucleic acid encoding a viral env to produce a packaging cell. Introducing a vector providing a heterologous gene, herein identified as a transfer vector, into that packaging cell yields a producer cell which releases infectious viral particles carrying the foreign gene of interest.

According to the above-indicated configuration of vectors and foreign genes, the second vector can provide a nucleic acid encoding a viral envelope (env) gene. The env gene can be derived from nearly any suitable virus, including retroviruses. In some embodiments, the env protein is an amphotropic envelope protein which allows transduction of cells of human and other species.

Examples of retroviral-derived env genes include, but are not limited to: Moloney murine leukemia virus (MoMuLV or MMLV), Harvey murine sarcoma virus (HaMuSV or HSV), murine mammary tumor virus (MuMTV or MMTV), gibbon ape leukemia virus (GaLV or GALV), human immunodeficiency virus (HIV) and Rous sarcoma virus (RSV). Other env genes such as Vesicular stomatitis virus (VSV) protein G (VSV G), that of hepatitis viruses and of influenza also can be used. In some embodiments, the viral env nucleic acid sequence is associated operably with regulatory sequences described elsewhere herein.

In certain embodiments, the lentiviral vector has the HIV virulence genes env, vif, vpr, vpu and nef deleted without compromising the ability of the vector to transduce non-dividing cells. In some embodiments, the lentiviral vector comprises a deletion of the U3 region of the 3′ LTR. The deletion of the U3 region can be the complete deletion or a partial deletion.

In some embodiments, the lentiviral vector of the disclosure comprising the FVIII nucleotide sequence described herein can be transfected in a cell with (a) a first nucleotide sequence comprising a gag, a pol, or gag and pol genes and (b) a second nucleotide sequence comprising a heterologous env gene; wherein the lentiviral vector lacks a functional tat gene. In other embodiments, the cell is further transfected with a fourth nucleotide sequence comprising a rev gene. In certain embodiments, the lentiviral vector lacks functional genes selected from vif, vpr, vpu, vpx and nef, or a combination thereof.

In certain embodiments, a lentiviral vector of the instant disclosure comprises one or more nucleotide sequences encoding a gag protein, a Rev-response element, a central polypurine track (cPPT), or any combination thereof.

In some embodiments, the lentiviral vector expresses on its surface one or more polypeptides that improve the targeting and/or activity of the lentiviral vector or the encoded FVIII polypeptide. The one or more polypeptides can be encoded by the lentiviral vector or can be incorporated during budding of the lentiviral vector from a host cell. During lentiviral production, viral particles bud off from a producing host cell. During the budding process, the viral particle takes on a lipid coat, which is derived from the lipid membrane of the host cell. As a result, the lipid coat of the viral particle can include membrane bound polypeptides that were previously present on the surface of the host cell.

In some embodiments, the lentiviral vector expresses one or more polypeptides on its surface that inhibit an immune response to the lentiviral vector following administration to a human subject. In some embodiments, the surface of the lentiviral vector comprises one or more CD47 molecules. CD47 is a “marker of self” protein, which is ubiquitously expressed on human cells. Surface expression of CD47 inhibits macrophage-induced phagocytosis of endogenous cells through the interaction of CD47 and macrophage expressed-SIRPα. Cells expressing high levels of CD47 are less likely to be targeted and destroyed by human macrophages in vivo.

In some embodiments, the lentiviral vector comprises a high concentration of CD47 polypeptide molecules on its surface. In some embodiments, the lentiviral vector is produced in a cell line that has a high expression level of CD47. In certain embodiments, the lentiviral vector is produced in a CD47high cell, wherein the cell has high expression of CD47 on the cell membrane. In particular embodiments, the lentiviral vector is produced in a CD47high HEK 293T cell, wherein the HEK 293T is has high expression of CD47 on the cell membrane. In some embodiments, the HEK 293T cell is modified to have increased expression of CD47 relative to unmodified HEK 293T cells. In certain embodiments, the CD47 is human CD47.

In some embodiments, the lentiviral vector has little or no surface expression of major histocompatibility complex class I (MHC-I). Surface expressed MHC-I displays peptide fragments of “non-self” proteins from within a cell, such as protein fragments indicative of an infection, facilitating an immune response against the cell. In some embodiments, the lentiviral vector is produced in a MHC-I^{low} cell, wherein the cell has reduced expression of MHC-I on the cell membrane. In some embodiments, the lentiviral vector is produced in an MHC-I- (or “MHC-I^{free}”, “MHC-1^{neg}” or “MHC-negative”) cell, wherein the cell lacks expression of MHC-I.

In particular embodiments, the lentiviral vector comprises a lipid coat comprising a high concentration of CD47 polypeptides and lacking MHC-I polypeptides. In certain embodiments, the lentiviral vector is produced in a CD47high/MHC-I^{low} cell line, e.g., a CD47high/MHC-I^{low} HEK 293T cell line. In some embodiments, the lentiviral vector is produced in a CD47high/MHC-Ifree cell line, e.g., a CD47high/MHC-I^{free} HEK 293T cell line.

Examples of lentiviral vectors are disclosed in U.S. Pat. No. 9,050,269 and International Publication Nos. WO9931251, WO9712622, WO9817815, WO9817816, and WO9818934, which are incorporated herein by reference in their entireties.

### Inverted Terminal Repeat (ITR) Sequences

In some embodiments, the nucleic acid sequences disclosed herein comprise inverted terminal repeat (ITR) sequences. As used herein, an “inverted terminal repeat” (or “ITR”) refers to a nucleic acid subsequence located at either the 5′ or 3′ end of a single stranded nucleic acid sequence, which comprises a set of nucleotides (initial sequence) followed downstream by its reverse complement, i.e., palindromic sequence. The intervening sequence of nucleotides between the initial sequence and the reverse complement can be any length including zero. In one embodiment, the ITR useful for the present disclosure comprises one or more “palindromic sequences.” An ITR can have any number of functions. In some embodiments, an ITR described herein forms a hairpin structure. In some embodiments, the ITR forms a T-shaped hairpin structure. In some embodiments, the ITR forms a non-T-shaped hairpin structure, e.g., a U-shaped hairpin structure. In some embodiments, the ITR promotes the long-term survival of the nucleic acid molecule in the nucleus of a cell. In some embodiments, the ITR promotes the permanent survival of the nucleic acid molecule in the nucleus of a cell (e.g., for the entire life-span of the cell). In some embodiments, the ITR promotes the stability of the nucleic acid molecule in the nucleus of a cell. In some embodiments, the ITR promotes the retention of the nucleic acid molecule in the nucleus of a cell. In some embodiments, the ITR promotes the persistence of the nucleic acid molecule in the nucleus of a cell. In some embodiments, the ITR inhibits or prevents the degradation of the nucleic acid molecule in the nucleus of a cell.

Therefore, an “ITR” as used herein can fold back on itself and form a double stranded segment. For example, the sequence GATCXXXXGATC comprises an initial sequence of GATC and its complement (3′CTAG5′) when folded to form a double helix. In some embodiments, the ITR comprises a continuous palindromic sequence (e.g., GATCGATC) between the initial sequence and the reverse complement. In some embodiments, the ITR comprises an interrupted palindromic sequence (e.g., GATCXXXXGATC) between the initial sequence and the reverse complement. In some embodiments, the complementary sections of the continuous or interrupted palindromic sequence interact with each other to form a “hairpin loop” structure. As used herein, a “hairpin loop” structure results when at least two complimentary sequences on a single-stranded nucleotide molecule base-pair to form a double stranded section. In some embodiments, only a portion of the ITR forms a hairpin loop. In other embodiments, the entire ITR forms a hairpin loop.

In the present disclosure, at least one ITR is an ITR of a non-adenovirus associated virus (non-AAV). In certain embodiments, the ITR is an ITR of a non-AAV member of the viral family Parvoviridae. In some embodiments, the ITR is an ITR of a non-AAV member of the genus

### Dependovirus or the Genus Erythrovirus.

In some embodiments, an ITR in a nucleic acid molecule described herein may be a transcriptionally activated ITR. A transcriptionally-activated ITR can comprise all or a portion of a wild-type ITR that has been transcriptionally activated by inclusion of at least one transcriptionally active element. Various types of transcriptionally active elements are suitable for use in this context. In some embodiments, the transcriptionally active element is a constitutive transcriptionally active element. Constitutive transcriptionally active elements provide an ongoing level of gene transcription, and are preferred when it is desired that the transgene be expressed on an ongoing basis. In other embodiments, the transcriptionally active element is an inducible transcriptionally active element. Inducible transcriptionally active elements generally exhibit low activity in the absence of an inducer (or inducing condition), and are up-regulated in the presence of the inducer (or switch to an inducing condition). Inducible transcriptionally active elements may be preferred when expression is desired only at certain times or at certain locations, or when it is desirable to titrate the level of expression using an inducing agent. Transcriptionally active elements can also be tissue-specific; that is, they exhibit activity only in certain tissues or cell types.

Transcriptionally active elements, can be incorporated into an ITR in a variety of ways. In some embodiments, a transcriptionally active element is incorporated 5′ to any portion of an ITR or 3′ to any portion of an ITR. In other embodiments, a transcriptionally active element of a transcriptionally-activated ITR lies between two ITR sequences. If the transcriptionally active element comprises two or more elements which must be spaced apart, those elements may alternate with portions of the ITR. In some embodiments, a hairpin structure of an ITR is deleted and replaced with inverted repeats of a transcriptional element. This latter arrangement would create a hairpin mimicking the deleted portion in structure. Multiple tandem transcriptionally active elements can also be present in a transcriptionally-activated ITR, and these may be adjacent or spaced apart. In addition, protein binding sites (e.g., Rep binding sites) can be introduced into transcriptionally active elements of the transcriptionally-activated ITRs. A transcriptionally active element can comprise any sequence enabling the controlled transcription of DNA by RNA polymerase to form RNA, and can comprise, for example, a transcriptionally active element, as defined below.

Transcriptionally-activated ITRs provide both transcriptional activation and ITR functions to the nucleic acid molecule in a relatively limited nucleotide sequence length which effectively maximizes the length of a transgene which can be carried and expressed from the nucleic acid molecule. Incorporation of a transcriptionally active element into an ITR can be accomplished in a variety of ways. A comparison of the ITR sequence and the sequence requirements of the transcriptionally active element can provide insight into ways to encode the element within an ITR. For example, transcriptional activity can be added to an ITR through the introduction of specific changes in the ITR sequence that replicates the functional elements of the transcriptionally active element. A number of techniques exist in the art to efficiently add, delete, and/or change particular nucleotide sequences at specific sites (see, for example, Deng and Nickoloff (1992) Anal. Biochem. 200:81-88). Another way to create transcriptionally-activated ITRs involves the introduction of a restriction site at a desired location in the ITR. In addition, multiple transcriptionally activate elements can be incorporated into a transcriptionally-activated ITR, using methods known in the art.

By way of illustration, transcriptionally-activated ITRs can be generated by inclusion of one or more transcriptionally active elements such as: TATA box, GC box, CCAAT box, Sp1 site, Inr region, CRE (cAMP regulatory element) site, ATF-1/CRE site, APBβ box, APBα box, CArG box, CCAC box, or any other element involved in transcription as known in the art.

### Host Cells

The disclosure also provides a host cell comprising a nucleic acid molecule or vector of the disclosure. As used herein, the term “transformation” shall be used in a broad sense to refer to the introduction of DNA into a recipient host cell that changes the genotype and consequently results in a change in the recipient cell.

“Host cells” refers to cells that have been transformed with vectors constructed using recombinant DNA techniques and encoding at least one heterologous gene. The host cells of the present disclosure are preferably of mammalian origin; most preferably of human or mouse origin. Those skilled in the art are credited with ability to preferentially determine particular host cell lines which are best suited for their purpose. Exemplary host cell lines include, but are not limited to, CHO, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), P3.times.63-Ag3.653 (mouse myeloma), BFA-1c1BPT (bovine endothelial cells), RAJI (human lymphocyte), PER.C6®, NS0, CAP, BHK21, and HEK 293 (human kidney). In one particular embodiment, the host cell is selected from the group consisting of: a CHO cell, a HEK293 cell, a BHK21 cell, a PER.C6® cell, a NS0 cell, and a CAP cell. Host cell lines are typically available from commercial services, the American Tissue Culture Collection, or from published literature.

Introduction of the isolated nucleic acid molecules or vectors of the disclosure into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. Sec, Ridgway, A. A. G. “Mammalian Expression Vectors” Chapter 24.2, pp. 470-472 Vectors, Rodriguez and Denhardt, Eds. (Butterworths, Boston, Mass. 1988). Plasmids can be introduced into the host via electroporation. The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or flourescence-activated cell sorter analysis (FACS), immunohistochemistry and the like.

Host cells comprising the isolated nucleic acid molecules or vectors of the disclosure are grown in an appropriate growth medium. As used herein, the term “appropriate growth medium” means a medium containing nutrients required for the growth of cells. Nutrients required for cell growth can include a carbon source, a nitrogen source, essential amino acids, vitamins, minerals, and growth factors. Optionally, the media can contain one or more selection factors. Optionally the media can contain bovine calf serum or fetal calf serum (FCS). In one embodiment, the media contains substantially no IgG. The growth medium will generally select for cells containing the DNA construct by, for example, drug selection or deficiency in an essential nutrient which is complemented by the selectable marker on the DNA construct or co-transfected with the DNA construct. Cultured mammalian cells are generally grown in commercially available serum-containing or serum-free media (e.g., MEM, DMEM, DMEM/F12). In one embodiment, the medium is CDoptiCHO (Invitrogen, Carlsbad, CA.). In another embodiment, the medium is CD17 (Invitrogen, Carlsbad, CA.). Selection of a medium appropriate for the particular cell line used is within the level of those ordinary skilled in the art.

In some embodiments, host cells suitable for use in the present invention are of insect origin. In some embodiments, a suitable insect host cell includes, for example, a cell line isolated from Spodoptera frugiperda (Sf) or a cell line isolated from Trichoplusia ni (Tni). Those of skill in the art will readily be able to determine the suitability of any Sf or Tni cell line. Exemplary insect host cells include, without limitation, Sf9 cells, Sf21 cells, and High Five™ cells. Exemplary insect host cells also include, without limitation, any Sf or Tni cell line that is free from adventitious virus contamination, e.g., Sf-rhabdovirus-negative (Sf-RVN) and Tn-nodavirus-negative (Tn-NVN) cells. Other suitable host insect cells are known to those of skill in the art. In one particular embodiment, the insect host cells are Sf9 cells.

Aspects of the present disclosure provide a method of cloning a nucleic acid molecule described herein, comprising inserting a nucleic acid molecule capable of complex secondary structures into a suitable vector, and introducing the resulting vector into a suitable bacterial host strain. As known in the art, complex secondary structures (e.g., long palindromic regions) of nucleic acids may be unstable and difficult to clone in bacterial host strains. For example, nucleic acid molecules comprising a first ITR and a second ITR (e.g., non-AAV parvoviral ITRs, e.g., HBOV1 ITRs) of the present disclosure may be difficult to clone using conventional methodologies. Long DNA palindromes inhibit DNA replication and are unstable in the genomes of E. coli, Bacillus, Streptococcus, Streptomyces, S. cerevisiae, mice, and humans. These effects result from the formation of hairpin or cruciform structures by intrastrand base pairing. In E. coli the inhibition of DNA replication can be significantly overcome in SbcC or SbcD mutants. SbcD is the nuclease subunit, and SbcC is the ATPase subunit of the SbcCD complex. The E. coli SbcCD complex is an exonuclease complex responsible for preventing the replication of long palindromes. The SbcCD complex is a nuclear with ATP-dependent double-stranded DNA exonuclease activity and ATP-independent single-stranded DNA endonuclease activity. SbcCD may recognize DNA palindromes and collapse replication forks by attacking hairpin structures that arise.

In certain embodiments, a suitable bacterial host strain is incapable of resolving cruciform DNA structures. In certain embodiments, a suitable bacterial host strain comprises a disruption in the SbcCD complex. In some embodiments, the disruption in the SbcCD complex comprises a genetic disruption in the SbcC gene and/or SbcD gene. In certain embodiments, the disruption in the SbcCD complex comprises a genetic disruption in the SbcC gene. Various bacterial host strains that comprise a genetic disruption in the SbcC gene are known in the art. For example, without limitation, the bacterial host strain PMC103 comprises the genotype sbcC, recD, mcrA, ΔmcrBCF; the bacterial host strain PMC107 comprises the genotype recBC, recJ, sbcBC, mcrA, ΔmcrBCF; and the bacterial host strain SURE comprises the genotype recB, recJ, sbcC, mcrA, ΔmcrBCF, umuC, uvrC. Accordingly, in some embodiments a method of cloning a nucleic acid molecule described herein comprises inserting a nucleic acid molecule capable of complex secondary structures into a suitable vector, and introducing the resulting vector into host strain PMC103, PMC107, or SURE. In certain embodiments, the method of cloning a nucleic acid molecule described herein comprises inserting a nucleic acid molecule capable of complex secondary structures into a suitable vector, and introducing the resulting vector into host strain PMC103.

Suitable vectors are known in the art and described elsewhere herein. In certain embodiments, a suitable vector for use in a cloning methodology of the present disclosure is a low copy vector. In certain embodiments, a suitable vector for use in a cloning methodology of the present disclosure is pBR322.

### Production of Polypeptides

The disclosure also provides a polypeptide encoded by a nucleic acid molecule of the disclosure. In other embodiments, the polypeptide of the disclosure is encoded by a vector comprising the isolated nucleic molecules of the disclosure. In yet other embodiments, the polypeptide of the disclosure is produced by a host cell comprising the isolated nucleic molecules of the disclosure.

A variety of methods are available for recombinantly producing a FVIII protein from the optimized nucleic acid molecule of the disclosure. A polynucleotide of the desired sequence can be produced by de novo solid-phase DNA synthesis or by PCR mutagenesis of an earlier prepared polynucleotide. Oligonucleotide-mediated mutagenesis is one method for preparing a substitution, insertion, deletion, or alteration (e.g., altered codon) in a nucleotide sequence. For example, the starting DNA is altered by hybridizing an oligonucleotide encoding the desired mutation to a single-stranded DNA template. After hybridization, a DNA polymerase is used to synthesize an entire second complementary strand of the template that incorporates the oligonucleotide primer. In one embodiment, genetic engineering, e.g., primer-based PCR mutagenesis, is sufficient to incorporate an alteration, as defined herein, for producing a polynucleotide of the disclosure.

For recombinant protein production, an optimized polynucleotide sequence of the disclosure encoding the FVIII protein is inserted into an appropriate expression vehicle, i.e., a vector which contains the necessary elements for the transcription and translation of the inserted coding sequence, or in the case of an RNA viral vector, the necessary elements for replication and translation.

The polynucleotide sequence of the disclosure is inserted into the vector in proper reading frame. The expression vector is then transfected into a suitable target cell which will express the polypeptide. Transfection techniques known in the art include, but are not limited to, calcium phosphate precipitation (Wigler et al. 1978, Cell 14:725) and electroporation (Neumann et al. 1982, EMBO, J. 1:841). A variety of host-expression vector systems can be utilized to express the FVIII proteins described herein in eukaryotic cells. In one embodiment, the eukaryotic cell is an animal cell, including mammalian cells (e.g. HEK293 cells, PER.C6®, CHO, BHK, Cos, HeLa cells). A polynucleotide sequence of the disclosure can also code for a signal sequence that will permit the FVIII protein to be secreted. One skilled in the art will understand that while the FVIII protein is translated the signal sequence is cleaved by the cell to form the mature protein. Various signal sequences are known in the art, e.g., native factor Vll signal sequence, native factor IX signal sequence and the mouse IgK light chain signal sequence. Alternatively, where a signal sequence is not included the FVIII protein can be recovered by lysing the cells.

The FVIII protein of the disclosure can be synthesized in a transgenic animal, such as a rodent, goat, sheep, pig, or cow. The term “transgenic animals” refers to non-human animals that have incorporated a foreign gene into their genome. Because this gene is present in germline tissues, it is passed from parent to offspring. Exogenous genes are introduced into single-celled embryos (Brinster et al. 1985, Proc. Natl. Acad. Sci. USA 82:4438). Methods of producing transgenic animals are known in the art including transgenics that produce immunoglobulin molecules (Wagner et al. 1981, Proc. Natl. Acad. Sci. USA 78:6376; Mcknight et al. 1983, Cell 34:335; Brinster et al. 1983, Nature 306:332; Ritchie et al. 1984, Nature 312:517; Baldassarre et al. 2003, Theriogenology 59:831; Robl et al. 2003, Theriogenology 59:107; Malassagne et al. 2003, Xenotransplantation 10 (3): 267).

The expression vectors can encode for tags that permit for easy purification or identification of the recombinantly produced protein. Examples include, but are not limited to, vector pUR278 (Ruther et al. 1983, EMBO J. 2:1791) in which the FVIII protein described herein coding sequence can be ligated into the vector in frame with the lac Z coding region so that a hybrid protein is produced; pGEX vectors can be used to express proteins with a glutathione S-transferase (GST) tag. These proteins are usually soluble and can easily be purified from cells by adsorption to glutathione-agarose beads followed by elution in the presence of free glutathione. The vectors include cleavage sites (e.g., PreCission Protease (Pharmacia, Peapack, N. J.)) for easy removal of the tag after purification.

For the purposes of this disclosure, numerous expression vector systems can be employed. These expression vectors are typically replicable in the host organisms either as episomes or as an integral part of the host chromosomal DNA. Expression vectors can include expression control sequences including, but not limited to, promoters (e.g., naturally-associated or heterologous promoters), enhancers, signal sequences, splice signals, enhancer elements, and transcription termination sequences. Preferably, the expression control sequences are eukaryotic promoter systems in vectors capable of transforming or transfecting eukaryotic host cells. Expression vectors can also utilize DNA elements which are derived from animal viruses such as bovine papilloma virus, polyoma virus, adenovirus, vaccinia virus, baculovirus, retroviruses (RSV, MMTV or MOMLV), cytomegalovirus (CMV), or SV40 virus. Others involve the use of polycistronic systems with internal ribosome binding sites.

Commonly, expression vectors contain selection markers (e.g., ampicillin-resistance, hygromycin-resistance, tetracycline resistance or neomycin resistance) to permit detection of those cells transformed with the desired DNA sequences (see, e.g., Itakura et al., U.S. Pat. No. 4,704,362). Cells which have integrated the DNA into their chromosomes can be selected by introducing one or more markers which allow selection of transfected host cells. The marker can provide for prototrophy to an auxotrophic host, biocide resistance (e.g., antibiotics) or resistance to heavy metals such as copper. The selectable marker gene can either be directly linked to the DNA sequences to be expressed, or introduced into the same cell by co-transformation.

An example of a vector useful for expressing an optimized FVIII sequence is NEOSPLA (U.S. Pat. No. 6,159,730). This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. This vector has been found to result in very high level expression of antibodies upon incorporation of variable and constant region genes, transfection in cells, followed by selection in G418 containing medium and methotrexate amplification. Vector systems are also taught in U.S. Pat. Nos. 5,736,137 and 5,658,570, each of which is incorporated by reference in its entirety herein. This system provides for high expression levels, e.g., >30 pg/cell/day. Other exemplary vector systems are disclosed e.g., in U.S. Pat. No. 6,413,777.

In other embodiments the polypeptides of the disclosure of the instant disclosure can be expressed using polycistronic constructs. In these expression systems, multiple gene products of interest such as multiple polypeptides of multimer binding protein can be produced from a single polycistronic construct. These systems advantageously use an internal ribosome entry site (IRES) to provide relatively high levels of polypeptides in eukaryotic host cells. Compatible IRES sequences are disclosed in U.S. Pat. No. 6,193,980 which is also incorporated herein.

More generally, once the vector or DNA sequence encoding a polypeptide has been prepared, the expression vector can be introduced into an appropriate host cell. That is, the host cells can be transformed. Introduction of the plasmid into the host cell can be accomplished by various techniques well known to those of skill in the art, as discussed above. The transformed cells are grown under conditions appropriate to the production of the FVIII polypeptide, and assayed for FVIII polypeptide synthesis. Exemplary assay techniques include enzyme-linked immunosorbent assay (ELISA), radioimmunoassay (RIA), or fluorescence-activated cell sorter analysis (FACS), immunohistochemistry and the like.

In descriptions of processes for isolation of polypeptides from recombinant hosts, the terms “cell” and “cell culture” are used interchangeably to denote the source of polypeptide unless it is clearly specified otherwise. In other words, recovery of polypeptide from the “cells” can mean either from spun down whole cells, or from the cell culture containing both the medium and the suspended cells.

The host cell line used for protein expression is preferably of mammalian origin; most preferably of human or mouse origin, as the isolated nucleic acids of the disclosure have been optimized for expression in human cells. Exemplary host cell lines have been described above. In one embodiment of the method to produce a polypeptide with FVIII activity, the host cell is a HEK293 cell. In another embodiment of the method to produce a polypeptide with FVIII activity, the host cell is a CHO cell.

Genes encoding the polypeptides of the disclosure can also be expressed in non-mammalian cells such as bacteria or yeast or plant cells. In this regard it will be appreciated that various unicellular non-mammalian microorganisms such as bacteria can also be transformed; i.e., those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the polypeptides typically become part of inclusion bodies. The polypeptides must be isolated, purified and then assembled into functional molecules.

Alternatively, optimized nucleotide sequences of the disclosure can be incorporated in transgenes for introduction into the genome of a transgenic animal and subsequent expression in the milk of the transgenic animal (see, e.g., Deboer et al., U.S. Pat. No. 5,741,957, Rosen, U.S. Pat. No. 5,304,489, and Meade et al., U.S. Pat. No. 5,849,992). Suitable transgenes include coding sequences for polypeptides in operable linkage with a promoter and enhancer from a mammary gland specific gene, such as casein or beta lactoglobulin.

In vitro production allows scale-up to give large amounts of the desired polypeptides. Techniques for mammalian cell cultivation under tissue culture conditions are known in the art and include homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose or (immuno-) affinity chromatography, e.g., after preferential biosynthesis of a synthetic hinge region polypeptide or prior to or subsequent to the HIC chromatography step described herein. An affinity tag sequence (e.g. a His (6) tag) can optionally be attached or included within the polypeptide sequence to facilitate downstream purification.

Once expressed, the FVIII protein can be purified according to standard procedures of the art, including ammonium sulfate precipitation, affinity column chromatography, HPLC purification, gel electrophoresis and the like (see generally Scopes, Protein Purification (Springer-Verlag, N.Y., (1982)). Substantially pure proteins of at least about 90 to 95% homogeneity are preferred for pharmaceutical uses, with 98 to 99% or more homogeneity being most preferred.

### Pharmaceutical Compositions

Compositions containing an isolated nucleic acid molecule, a polypeptide having FVIII activity encoded by the nucleic acid molecule, a vector, or a host cell of the present disclosure can contain a suitable pharmaceutically acceptable carrier. For example, they can contain excipients and/or auxiliaries that facilitate processing of the active compounds into preparations designed for delivery to the site of action.

The pharmaceutical composition can be formulated for parenteral administration (i.e. intravenous, subcutaneous, or intramuscular) by bolus injection. Formulations for injection can be presented in unit dosage form, e.g., in ampoules or in multidose containers with an added preservative. The compositions can take such forms as suspensions, solutions, or emulsions in oily or aqueous vehicles, and contain formulatory agents such as suspending, stabilizing and/or dispersing agents. Alternatively, the active ingredient can be in powder form for constitution with a suitable vehicle, e.g., pyrogen free water.

Suitable formulations for parenteral administration also include aqueous solutions of the active compounds in water-soluble form, for example, water-soluble salts. In addition, suspensions of the active compounds as appropriate oily injection suspensions can be administered. Suitable lipophilic solvents or vehicles include fatty oils, for example, sesame oil, or synthetic fatty acid esters, for example, ethyl oleate or triglycerides. Aqueous injection suspensions can contain substances, which increase the viscosity of the suspension, including, for example, sodium carboxymethyl cellulose, sorbitol and dextran. Optionally, the suspension can also contain stabilizers. Liposomes also can be used to encapsulate the molecules of the disclosure for delivery into cells or interstitial spaces. Exemplary pharmaceutically acceptable carriers are physiologically compatible solvents, dispersion media, coatings, antibacterial and antifungal agents, isotonic and absorption delaying agents, water, saline, phosphate buffered saline, dextrose, glycerol, ethanol and the like. In some embodiments, the composition comprises isotonic agents, for example, sugars, polyalcohols such as mannitol, sorbitol, or sodium chloride. In other embodiments, the compositions comprise pharmaceutically acceptable substances such as wetting agents or minor amounts of auxiliary substances such as wetting or emulsifying agents, preservatives or buffers, which enhance the shelf life or effectiveness of the active ingredients.

Compositions of the disclosure can be in a variety of forms, including, for example, liquid (e.g., injectable and infusible solutions), dispersions, suspensions, semi-solid and solid dosage forms. The preferred form depends on the mode of administration and therapeutic application.

The composition can be formulated as a solution, micro emulsion, dispersion, liposome, or other ordered structure suitable to high drug concentration. Sterile injectable solutions can be prepared by incorporating the active ingredient in the required amount in an appropriate solvent with one or a combination of ingredients enumerated above, as required, followed by filtered sterilization. Generally, dispersions are prepared by incorporating the active ingredient into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The active ingredient can be formulated with a controlled-release formulation or device. Examples of such formulations and devices include implants, transdermal patches, and microencapsulated delivery systems. Biodegradable, biocompatible polymers can be used, for example, ethylene vinyl acetate, polyanhydrides, polyglycolic acid, collagen, polyorthoesters, and polylactic acid. Methods for the preparation of such formulations and devices are known in the art. See, e.g., Sustained and Controlled Release Drug Delivery Systems, J. R. Robinson, ed., Marcel Dekker, Inc., New York, 1978.

Injectable depot formulations can be made by forming microencapsulated matrices of the drug in biodegradable polymers such as polylactide-polyglycolide. Depending on the ratio of drug to polymer, and the nature of the polymer employed, the rate of drug release can be controlled. Other exemplary biodegradable polymers are polyorthoesters and polyanhydrides. Depot injectable formulations also can be prepared by entrapping the drug in liposomes or microemulsions.

Supplementary active compounds can be incorporated into the compositions. In one embodiment, the chimeric protein of the disclosure is formulated with another clotting factor, or a variant, fragment, analogue, or derivative thereof. For example, the clotting factor includes, but is not limited to, factor V, factor VII, factor VIII, factor IX, factor X, factor XI, factor XII, factor XIII, prothrombin, fibrinogen, von Willebrand factor or recombinant soluble tissue factor (rsTF) or activated forms of any of the preceding. The clotting factor of hemostatic agent can also include anti-fibrinolytic drugs, e.g., epsilon-amino-caproic acid, tranexamic acid.

Dosage regimens can be adjusted to provide the optimum desired response. For example, a single bolus can be administered, several divided doses can be administered over time, or the dose can be proportionally reduced or increased as indicated by the exigencies of the therapeutic situation. It is advantageous to formulate parenteral compositions in dosage unit form for case of administration and uniformity of dosage. Sec, e.g., Remington's Pharmaceutical Sciences (Mack Pub. Co., Easton, Pa. 1980).

In addition to the active compound, the liquid dosage form can contain inert ingredients such as water, ethyl alcohol, ethyl carbonate, ethyl acetate, benzyl alcohol, benzyl benzoate, propylene glycol, 1,3-butylene glycol, dimethylformamide, oils, glycerol, tetrahydrofurfuryl alcohol, polyethylene glycols, and fatty acid esters of sorbitan.

Non-limiting examples of suitable pharmaceutical carriers are also described in Remington's Pharmaceutical Sciences by E. W. Martin. Some examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition can also contain pH buffering reagents, and wetting or emulsifying agents.

For oral administration, the pharmaceutical composition can take the form of tablets or capsules prepared by conventional means. The composition can also be prepared as a liquid for example a syrup or a suspension. The liquid can include suspending agents (e.g., sorbitol syrup, cellulose derivatives or hydrogenated edible fats), emulsifying agents (lecithin or acacia), non-aqueous vehicles (e.g., almond oil, oily esters, ethyl alcohol, or fractionated vegetable oils), and preservatives (e.g., methyl or propyl-p-hydroxybenzoates or sorbic acid). The preparations can also include flavoring, coloring and sweetening agents. Alternatively, the composition can be presented as a dry product for constitution with water or another suitable vehicle.

For buccal administration, the composition can take the form of tablets or lozenges according to conventional protocols.

For administration by inhalation, the compounds for use according to the present disclosure are conveniently delivered in the form of a nebulized aerosol with or without excipients or in the form of an aerosol spray from a pressurized pack or nebulizer, with optionally a propellant, e.g., dichlorodifluoromethane, trichlorofluoromethane, dichlorotetrafluoromethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The pharmaceutical composition can also be formulated for rectal administration as a suppository or retention enema, e.g., containing conventional suppository bases such as cocoa butter or other glycerides.

In one embodiment, a pharmaceutical composition comprises a polypeptide having Factor VIII activity, an optimized nucleic acid molecule encoding the polypeptide having Factor VIII activity, the vector comprising the nucleic acid molecule, or the host cell comprising the vector, and a pharmaceutically acceptable carrier. In some embodiments, the composition is administered by a route selected from the group consisting of topical administration, intraocular administration, parenteral administration, intrathecal administration, subdural administration and oral administration. The parenteral administration can be intravenous or subcutaneous administration.

### Methods of Treatment

In some aspects, the present disclosure is directed to methods of treating a disease or condition in a subject in need thereof, comprising administering a nucleic acid molecule, a vector, a polypeptide, or a pharmaceutical composition disclosed herein.

In some embodiments, the present disclosure is directed to methods for increasing expression of a polypeptide with FVIII activity in a subject. In some embodiments, the method comprises administering a nucleic acid molecule comprising a nucleotide sequence having at least 80% sequence identity to SEQ ID NO: 11, SEQ ID NO: 14, or SEQ ID NO: 16.

In some embodiments, the disclosure is directed to methods of treating a bleeding disorder. In some embodiments, the disclosure is directed to methods of treating hemophilia A.

The isolated nucleic acid molecule, vector, or polypeptide can be administered intravenously, subcutaneously, intramuscularly, or via any mucosal surface, e.g., orally, sublingually, buccally, sublingually, nasally, rectally, vaginally or via pulmonary route. The isolated nucleic acid molecule, vector, or polypeptide can also be administered intraneurally, intraocularly, and intrathecally. The clotting factor protein can be implanted within or linked to a biopolymer solid support that allows for the slow release of the chimeric protein to the desired site.

In one embodiment, the route of administration of the isolated nucleic acid molecule, vector, or polypeptide is parenteral. The term parenteral as used herein includes intravenous, intraarterial, intraperitoneal, intramuscular, subcutaneous, rectal or vaginal administration. In some embodiments, the isolated nucleic acid molecule, vector, or polypeptide is administered intravenously. While all these forms of administration are clearly contemplated as being within the scope of the disclosure, a form for administration would be a solution for injection, in particular for intravenous or intraarterial injection or drip.

Effective doses of the compositions of the present disclosure, for the treatment of conditions vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages can be titrated using routine methods known to those of skill in the art to optimize safety and efficacy.

The nucleic acid molecule, vector, or polypeptides of the disclosure can optionally be administered in combination with other agents that are effective in treating the disorder or condition in need of treatment (e.g., prophylactic or therapeutic).

As used herein, the administration of isolated nucleic acid molecules, vectors, or polypeptides of the disclosure in conjunction or combination with an adjunct therapy means the sequential, simultaneous, coextensive, concurrent, concomitant or contemporaneous administration or application of the therapy and the disclosed polypeptides. Those skilled in the art will appreciate that the administration or application of the various components of the combined therapeutic regimen can be timed to enhance the overall effectiveness of the treatment. A skilled artisan (e.g., a physician) would be readily be able to discern effective combined therapeutic regimens without undue experimentation based on the selected adjunct therapy and the teachings of the instant specification.

It will further be appreciated that the isolated nucleic acid molecule, vector, or polypeptide of the instant disclosure can be used in conjunction or combination with an agent or agents (e.g., to provide a combined therapeutic regimen). Exemplary agents with which a polypeptide or polynucleotide of the disclosure can be combined include agents that represent the current standard of care for a particular disorder being treated. Such agents can be chemical or biologic in nature. The term “biologic” or “biologic agent” refers to any pharmaceutically active agent made from living organisms and/or their products which is intended for use as a therapeutic.

The amount of agent to be used in combination with the polynucleotides or polypeptides of the instant disclosure can vary by subject or can be administered according to what is known in the art. See, e.g., Bruce A Chabner et al., Antineoplastic Agents, in GOODMAN & GILMAN'S THE PHARMACOLOGICAL BASIS OF THERAPEUTICS 1233-1287 ((Joel G. Hardman et al., eds., 9th ed. 1996). In another embodiment, an amount of such an agent consistent with the standard of care is administered.

In one embodiment, also disclosed herein is a kit, comprising the nucleic acid molecule disclosed herein and instructions for administering the nucleic acid molecule to a subject in need thereof. In another embodiment, disclosed herein is a baculovirus system for production of the nucleic acid molecule provided herein. The nucleic acid molecule is produced in insect cells. In another embodiment, a nanoparticle delivery system for expression constructs is provided. The expression construct comprises the nucleic acid molecule disclosed herein.

### Gene Therapy

Somatic gene therapy has been explored as a possible treatment for bleeding disorders, and in particular, hemophilia A. Gene therapy is a particularly appealing treatment for hemophilia because of its potential to cure the disease through continuous endogenous production of FVIII following a single administration of a vector encoding FVIII. Hemophilia A is well suited for a gene replacement approach because its clinical manifestations are entirely attributable to the lack of a single gene product (FVIII) that circulates in minute amounts (200 ng/ml) in the plasma.

Lentiviral vectors are gaining prominence as gene delivery vehicles due to their large capacity and ability to sustain transgene expression via integration. Lentiviral vectors have been evaluated in numerous ex-vivo cell therapy clinical programs with promising efficacy and safety profiles.

The present disclosure meets an important need in the art by providing lentiviral vectors comprising a codon optimized FVIII sequence that demonstrates increased expression in a subject and potentially results in greater therapeutic efficacy when used in gene therapy methods. Embodiments of the present disclosure are directed to lentiviral vectors comprising one or more codon optimized nucleic acid molecules encoding a polypeptide with FVIII activity described herein, host cells (e.g., hepatocytes) comprising the lentiviral vectors, and methods of use of the disclosed lentiviral vectors (e.g., treatments for bleeding disorders using the lentiviral vectors disclosed herein).

In general, the methods of treatment disclosed herein involve administration of a lentiviral vector comprising a nucleic acid molecule comprising at least one codon optimized nucleic acid sequence encoding a FVIII clotting factor, wherein the nucleic acid sequence encoding a FVIII clotting factor is operably linked to suitable expression control sequences, which in some embodiments are incorporated into the lentiviral vector (e.g., a replication-defective lentiviral viral vector).

The present disclosure provides methods of treating a bleeding disorder (e.g., hemophilia A) in a subject in need thereof comprising administering to the subject at least one dose of 5×10¹⁰ or less transducing units/kg (TU/kg) (or 10⁹ or less TU/kg, or 10⁸ or less TU/kg) of a lentiviral vector comprising an isolated nucleic acid molecule comprising a nucleotide sequence encoding a polypeptide with FVIII activity. In some embodiments, the nucleotide sequence has at least 50%, at least 55%, at least 60%, at least 65%, at least 70%, at least 75%, at least 80%, at least 85%, at least 90%, at least 95%, at least 96%, at least 97%, at least 98%, at least 99%, or 100% sequence identity to SEQ ID NO: 11. In some embodiments, the nucleotide sequence has at least 50%, at least 55%, at least 60%, at least 65%, at least 70%, at least 75%, at least 80%, at least 85%, at least 90%, at least 95%, at least 96%, at least 97%, at least 98%, at least 99%, or 100% sequence identity to SEQ ID NO: 14.

In some embodiments, the lentiviral vector is administered as a single dose or multiple doses. In some embodiments, the lentiviral vector dose is administered at once or divided into multiple sub-dose, e.g., two sub-doses, three sub-doses, four sub-doses, five sub-doses, six sub-doses, or more than six sub-doses. In some embodiments, more than one lentiviral vector is administered.

In some embodiments, the dose of lentiviral vector is administered repeated at least twice, at least three times, at least four times, at least five times, at least six times, at least seven times, at least eight times, at least nine times, or at least ten times. In some embodiments, the lentiviral vector is administered via intravenous injection.

In some embodiments, the subject is a pediatric subject, whereas in other aspects, the subject is an adult subject.

In some embodiments, the lentiviral vector comprises at least one tissue specific promoter, i.e., a promoter that would regulate the expression of the polypeptide with FVIII activity in a particular tissue or cell type. In some embodiments, a tissue specific promoter in the lentiviral vector selectively enhances expression of the polypeptide with FVIII activity in a target liver cell. In some embodiments, the tissue specific promoter that selectively enhances expression of the polypeptide with FVIII activity in a target liver cell comprises an mTTR promoter. In some embodiments, the target liver cell is a hepatocyte.

Since the lentiviral vector can transduce all liver cell types, the expression of the transgene (e.g., FVIII) in different cell types can be controlled by using different promoters in the lentiviral vector. Thus, the lentiviral vector can comprise specific promoters which would control expression of the FVIII transgene in different tissues or cells types, such as different hepatic tissues or cell types. Thus, in some embodiments, the lentiviral vector can comprise an endothelial specific promoter which would control expression of the FVIII transgene in hepatic endothelial tissue, or a hepatocyte specific promoter which would control expression of the FVIII transgene in hepatocytes, or both.

In some embodiments, the lentiviral vector comprises a tissue-specific promoter or tissue-specific promoters that control the expression of the FVIII transgene in tissues other than liver. In some embodiments, the isolated nucleic acid molecule is stably integrated into the genome of the target cell or target tissue, for example, in the genome of a hepatocyte or in the genome of a hepatic endothelial cell.

In some embodiments, the nucleotide sequence encoding a polypeptide with FVIII activity in the lentivirus vector of the present disclosure comprises, consists, or consists essentially of coBDDFVIII-3aa (SEQ ID NO:14).

In other embodiments, the nucleotide sequence encoding a polypeptide with FVIII activity in the lentivirus vector of the present disclosure comprises, consists, or consists essentially of coBDDFVIII6-XTEN-3aa (SEQ ID NO:11).

In other embodiments, the nucleotide sequence encoding a polypeptide with FVIII activity in the lentivirus vector of the present disclosure comprises, consists, or consists essentially of SEQ ID NO:16.

The lentiviral vectors disclosed herein can be used at low dosages (e.g., 10¹⁰ TU/kg or lower, 10⁹ TU/kg or lower, or 10⁸ TU/kg or lower) in vivo in a mammal, e.g., a human patient, using a gene therapy approach to treatment of a bleeding disease or disorder selected from the group consisting of a bleeding coagulation disorder, hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, and bleeding in the illiopsoas sheath would be therapeutically beneficial. In one embodiment, the bleeding disease or disorder is hemophilia. In another embodiment, the bleeding disease or disorder is hemophilia A.

In some embodiments, target cells (e.g., hepatocytes) are treated in vitro with low doses (e.g., 10¹⁰ TU/kg or lower, 10⁹ TU/kg or lower, or 10⁸ TU/kg or lower) of the lentiviral vectors disclosed herein before being administered to the patient. In certain embodiments, target cells (e.g., hepatocytes) are treated in vitro with about 3.0×10⁹ TU/kg of the lentiviral vectors disclosed herein before being administered to the patient. In yet another embodiment, cells from the patient (e.g., hepatocytes) are treated ex vivo with low doses (e.g., 10¹⁰ TU/kg or lower, 10⁹ TU/kg or lower, or 10⁸ TU/kg or lower) of the lentiviral vectors disclosed herein before being administered to the patient.

In some embodiments, plasma FVIII activity post administration of a lentiviral vectors disclosed herein (administered, e.g., at 10¹⁰ TU/kg or lower, 10⁹ TU/kg or lower, or 10⁸ TU/kg or lower) is increased by at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300%, relative to physiologically normal circulating FVIII levels.

The present disclosure also provides methods of treating, preventing, r ameliorating a hemostatic disorder (e.g., a bleeding disorder such as hemophilia A) in a subject in need thereof comprising administering to the subject a therapeutically effective amount of a lentiviral vector comprising an isolated nucleic acid molecule comprising a nucleotide sequence encoding a polypeptide with FVIII activity, wherein the lentiviral vector is administered as at least one dose of 5×10¹⁰ or less TU/kg, 10⁹ or less TU/kg, or 10⁸ or less TU/kg.

The treatment, amelioration, and prevention by the lentiviral vector of the present disclosure can be a bypass therapy. The subject receiving bypass therapy can have already developed an inhibitor to a clotting factor, e.g., FVIII, or is subject to developing a clotting factor inhibitor.

The lentiviral vectors of the present disclosure treat or prevent a hemostatic disorder by promoting the formation of a fibrin clot. The polypeptide having FVIII activity encoded by the nucleic acid molecule of the disclosure can activate a member of a coagulation cascade. The clotting factor can be a participant in the extrinsic pathway, the intrinsic pathway or both.

The lentiviral vectors of the present disclosure can be used to treat hemostatic disorders known to be treatable with FVIII. The hemostatic disorders that can be treated using methods of the disclosure include, but are not limited to, hemophilia A, hemophilia B, von Willebrand's disease, Factor XI deficiency (PTA deficiency), Factor XII deficiency, as well as deficiencies or structural abnormalities in fibrinogen, prothrombin, Factor V, Factor VII, Factor X, or Factor XIII, hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, and bleeding in the illiopsoas sheath.

Compositions for administration to a subject include lentiviral vectors comprising nucleic acid molecules which comprise an optimized nucleotide sequence of the disclosure encoding a FVIII clotting factor (for gene therapy applications) as well as FVIII polypeptide molecules. In some embodiments, the composition for administration is a cell contacted with a lentiviral vector of the present disclosure, either in vivo, in vitro, or ex vivo.

In some embodiments, the hemostatic disorder is an inherited disorder. In one embodiment, the subject has hemophilia A. In other embodiments, the hemostatic disorder is the result of a deficiency in FVIII. In other embodiments, the hemostatic disorder can be the result of a defective FVIII clotting factor.

In another embodiment, the hemostatic disorder can be an acquired disorder. The acquired disorder can result from an underlying secondary disease or condition. The unrelated condition can be, as an example, but not as a limitation, cancer, an autoimmune disease, or pregnancy. The acquired disorder can result from old age or from medication to treat an underlying secondary disorder (e.g., cancer chemotherapy).

The disclosure also relates to methods of treating a subject that does not have a hemostatic disorder or a secondary disease or condition resulting in acquisition of a hemostatic disorder. The disclosure thus relates to a method of treating a subject in need of a general hemostatic agent comprising administering a therapeutically effective amount of a lentiviral vector of the present disclosure. For example, in one embodiment, the subject in need of a general hemostatic agent is undergoing, or is about to undergo, surgery. The lentiviral vector of the disclosure can be administered prior to or after surgery as a prophylactic.

The lentiviral vector of the disclosure can be administered during or after surgery to control an acute bleeding episode. The surgery can include, but is not limited to, liver transplantation, liver resection, or stem cell transplantation.

In another embodiment, the lentiviral vector of the disclosure can be used to treat a subject having an acute bleeding episode who does not have a hemostatic disorder. The acute bleeding episode can result from severe trauma, e.g., surgery, an automobile accident, wound, laceration gun shot, or any other traumatic event resulting in uncontrolled bleeding.

The lentiviral vector can be used to prophylactically treat a subject with a hemostatic disorder. The lentiviral vector can also be used to treat an acute bleeding episode in a subject with a hemostatic disorder.

In another embodiment, the administration of a lentiviral vector disclosed herein and/or subsequent expression of FVIII protein transgene does not induce an immune response in a subject. In some embodiments, the immune response comprises development of antibodies against FVIII. In some embodiments, the immune response comprises cytokine secretion. In some embodiments, the immune response comprises activation of B cells, T cells, or both B cells and T cells. In some embodiments, the immune response is an inhibitory immune response, wherein the immune response in the subject reduces the activity of the FVIII protein relative to the activity of the FVIII in a subject that has not developed an immune response. In certain embodiments, expression of FVIII protein by administering the lentiviral vector of the disclosure prevents an inhibitory immune response against the FVIII protein or the FVIII protein expressed from the isolated nucleic acid molecule or the lentiviral vector.

In some embodiments, a lentiviral vector of the disclosure is administered in combination with at least one other agent that promotes hemostasis. Said other agent that promotes hemostasis in a therapeutic with demonstrated clotting activity. As an example, but not as a limitation, the hemostatic agent can include Factor V, Factor VII, Factor IX, Factor X, Factor XI, Factor XII, Factor XIII, prothrombin, or fibrinogen or activated forms of any of the preceding. The clotting factor or hemostatic agent can also include anti-fibrinolytic drugs, e.g., epsilon-amino-caproic acid, tranexamic acid.

In one embodiment of the disclosure, the composition (e.g., the lentiviral vector) is one in which the FVIII is present in activatable form when administered to a subject. Such an activatable molecule can be activated in vivo at the site of clotting after administration to a subject.

The lentiviral vector of the disclosure can be administered intravenously, subcutaneously, intramuscularly, or via any mucosal surface, e.g., orally, sublingually, buccally, sublingually, nasally, rectally, vaginally or via pulmonary route. The lentiviral vector can be implanted within or linked to a biopolymer solid support that allows for the slow release of the vector to the desired site.

In one embodiment, the route of administration of the lentiviral vectors is parenteral. The term parenteral as used herein includes intravenous, intraarterial, intraperitoneal, intramuscular, subcutaneous, rectal or vaginal administration. The intravenous form of parenteral administration is preferred. While all these forms of administration are clearly contemplated as being within the scope of the disclosure, a form for administration would be a solution for injection, in particular for intravenous or intraarterial injection or drip. Usually, a suitable pharmaceutical composition for injection can comprise a buffer (e.g. acetate, phosphate or citrate buffer), a surfactant (e.g. polysorbate), optionally a stabilizer agent (e.g. human albumin), etc. However, in other methods compatible with the teachings herein, the lentiviral vector can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject disclosure, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a polypeptide by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations can be packaged and sold in the form of a kit. Such articles of manufacture will preferably have labels or package inserts indicating that the associated compositions are useful for treating a subject suffering from, or predisposed to clotting disorders.

The pharmaceutical composition can also be formulated for rectal administration as a suppository or retention enema, e.g., containing conventional suppository bases such as cocoa butter or other glycerides.

Effective doses of the compositions of the present disclosure, for the treatment of conditions vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages can be titrated using routine methods known to those of skill in the art to optimize safety and efficacy.

The lentiviral vector can be administered as a single dose or as multiple doses, wherein the multiple doses can be administered continuously or at specific timed intervals. In vitro assays can be employed to determine optimal dose ranges and/or schedules for administration. In vitro assays that measure clotting factor activity are known in the art. Additionally, effective doses can be extrapolated from dose-response curves obtained from animal models, e.g., a hemophiliac dog (Mount et al. 2002, Blood 99 (8): 2670).

Doses intermediate in the above ranges are also intended to be within the scope of the disclosure. Subjects can be administered such doses daily, on alternative days, weekly or according to any other schedule determined by empirical analysis. An exemplary treatment entails administration in multiple dosages over a prolonged period, for example, of at least six months.

The lentiviral vector of the disclosure can be administered on multiple occasions. Intervals between single dosages can be daily, weekly, monthly or yearly. Intervals can also be irregular as indicated by measuring blood levels of modified polypeptide or antigen in the patient. Dosage and frequency of the lentiviral vectors of the disclosure vary depending on the half-life of the FVIII polypeptide encoded by the transgene in the patient.

The dosage and frequency of administration of the lentiviral vectors of the disclosure can vary depending on whether the treatment is prophylactic or therapeutic. In prophylactic applications, compositions containing the lentiviral vector of the disclosure are administered to a patient not already in the disease state to enhance the patient's resistance or minimize effects of disease. Such an amount is defined to be a “prophylactic effective dose.” A relatively low dosage is administered at relatively infrequent intervals over a long period of time. Some patients continue to receive treatment for the rest of their lives.

The lentiviral vector of the disclosure can optionally be administered in combination with other agents that are effective in treating the disorder or condition in need of treatment (e.g., prophylactic or therapeutic).

As used herein, the administration of lentiviral vectors of the disclosure in conjunction or combination with an adjunct therapy means the sequential, simultaneous, coextensive, concurrent, concomitant or contemporaneous administration or application of the therapy and the disclosed polypeptides. Those skilled in the art will appreciate that the administration or application of the various components of the combined therapeutic regimen can be timed to enhance the overall effectiveness of the treatment. A skilled artisan (e.g., a physician) would be readily be able to discern effective combined therapeutic regimens without undue experimentation based on the selected adjunct therapy and the teachings of the instant specification.

It will further be appreciated that the lentiviral vectors of the disclosure can be used in conjunction or combination with an agent or agents (e.g., to provide a combined therapeutic regimen). Exemplary agents with which a lentiviral vector of the instant disclosure can be combined include agents that represent the current standard of care for a particular disorder being treated. Such agents can be chemical or biologic in nature. The term “biologic” or “biologic agent” refers to any pharmaceutically active agent made from living organisms and/or their products which is intended for use as a therapeutic.

The amount of agent to be used in combination with the lentiviral vectors of the instant disclosure can vary by subject or can be administered according to what is known in the art. Sec, e.g., Chabner et al., Pharmacological Basis of Therapeutics 1233-1287 (Joel G. Hardman et al., eds., 9th ed. 1996). In another embodiment, an amount of such an agent consistent with the standard of care is administered.

In certain embodiments, the lentiviral vectors of the present disclosure are administered in conjunction with an immunosuppressive, anti-allergic, or anti-inflammatory agent. These agents generally refer to substances that act to suppress or mask the immune system of the subject being treated herein. These agents include substances that suppress cytokine production, downregulate or suppress self-antigen expression, or mask the MHC antigens. Examples of such agents include 2-amino-6-aryl-5-substituted pyrimidines; azathioprine; cyclophosphamide; bromocryptinc; danazol; dapsone; glutaraldehyde; anti-idiotypic antibodies for MHC antigens and MHC fragments; cyclosporin A; steroids such as glucocorticosteroids, e.g., prednisone, methylprednisolone, and dexamethasone; cytokine or cytokine receptor antagonists including anti-interferon-γ, -β, or -α antibodies, anti-tumor necrosis factor-α antibodies, anti-tumor necrosis factor-β antibodies, anti-interleukin-2 antibodies and anti-IL-2 receptor antibodies; anti-LFA-1 antibodies, including anti-CD11a and anti-CD18 antibodies; anti-L3T4 antibodies; heterologous anti-lymphocyte globulin; pan-T antibodies; soluble peptide containing a LFA-3 binding domain; streptokinase; TGF-β; streptodornase; FK506; RS-61443; deoxyspergualin; and rapamycin. In certain embodiments, the agent is an antihistamine. An “antihistamine” as used herein is an agent that antagonizes the physiological effect of histamine. Examples of antihistamines are chlorpheniramine, diphenhydramine, promethazine, cromolyn sodium, astemizole, azatadine maleate, bropheniramine maleate, carbinoxamine maleate, cetirizine hydrochloride, clemastine fumarate, cyproheptadine hydrochloride, dexbrompheniramine maleate, dexchlorpheniramine maleate, dimenhydrinate, diphenhydramine hydrochloride, doxylamine succinate, fexofendadine hydrochloride, terphenadine hydrochloride, hydroxyzine hydrochloride, loratidine, meclizine hydrochloride, tripelannamine citrate, tripelennamine hydrochloride, and triprolidine hydrochloride.

Immunosuppressive, anti-allergic, or anti-inflammatory agents may be incorporated into the lentiviral vector administration regimen. For example, administration of immunosuppressive or anti-inflammatory agents may commence prior to administration of the disclosed lentiviral vectors, and may continue with one or more doses thereafter. In certain embodiments, the immunosuppressive or anti-inflammatory agents are administered as premedication to the lentiviral vectors.

As previously discussed, the lentiviral vectors of the present disclosure, can be administered in a pharmaceutically effective amount for the in vivo treatment of clotting disorders. In this regard, it will be appreciated that the lentiviral vectors of the disclosure can be formulated to facilitate administration and promote stability of the active agent. Preferably, pharmaceutical compositions in accordance with the present disclosure comprise a pharmaceutically acceptable, non-toxic, sterile carrier such as physiological saline, non-toxic buffers, preservatives and the like. Of course, the pharmaceutical compositions of the present disclosure can be administered in single or multiple doses to provide for a pharmaceutically effective amount of the polypeptide.

A number of tests are available to assess the function of the coagulation system: activated partial thromboplastin time (aPTT) test, chromogenic assay, ROTEM® assay, prothrombin time (PT) test (also used to determine INR), fibrinogen testing (often by the Clauss method), platelet count, platelet function testing (often by PFA-100), TCT, bleeding time, mixing test (whether an abnormality corrects if the patient's plasma is mixed with normal plasma), coagulation factor assays, antiphosholipid antibodies, D-dimer, genetic tests (e.g., factor V Leiden, prothrombin mutation G20210A), dilute Russell's viper venom time (dRVVT), miscellaneous platelet function tests, thromboclastography (TEG or Sonoclot), thromboelastometry (TEM®, e.g, ROTEM®), or euglobulin lysis time (ELT).

The aPTT test is a performance indicator measuring the efficacy of both the “intrinsic” (also referred to the contact activation pathway) and the common coagulation pathways. This test is commonly used to measure clotting activity of commercially available recombinant clotting factors, e.g., FVIII or FIX. It is used in conjunction with prothrombin time (PT), which measures the extrinsic pathway.

ROTEM® analysis provides information on the whole kinetics of haemostasis: clotting time, clot formation, clot stability and lysis. The different parameters in thromboelastometry are dependent on the activity of the plasmatic coagulation system, platelet function, fibrinolysis, or many factors which influence these interactions. This assay can provide a complete view of secondary haemostasis.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference.

Having generally described this disclosure, a further understanding can be obtained by reference to the examples provided herein. These examples are for purposes of illustration only and are not intended to be limiting.

### EXAMPLES

### Example 1: Generation of the Optimized coBDDFVIII6-XTEN-3Aa Transgene

It was hypothesized that transgene expression level can be increased by codon-optimizing the coding sequence for the targeted hosts. Higher level of FVIII expression has been demonstrated using a codon-optimized FVIIIco6XTEN genetic cassette in previous studies. This genetic cassette comprises a codon optimized cDNA encoding B-domain deleted human Factor VIII (BDDcoFVIII) fused with XTEN 144 peptide in the B-domain of FVIII.

To further improve the target specificity and reduce immunogenicity, an in silico antigenicity analysis was used to evaluate and minimize the risk of introducing neo-epitopes into the FVIIIco6XTEN protein. Various representative human leukocyte antigen (HLA) alleles (DR, DP, DQ) were evaluated using the open source Immune Epitope Database and Analysis Resource (IEDB) and recommended prediction method to determine major histocompatibility complex class II (MHCII) binding to the chimeric protein. For additional analysis, the NetMHCIIpan 3.0 method with HLA-DR alleles (representative of North American or Japanese populations) was also used (see Lamberth K, et al. Sci Transl Med. 2017; 9 (372): caag1286,).

Peptides with half maximal inhibitory concentration (IC₅₀) values <50 nM, <500 nM, <5000 nM were considered to have high (high immunogenic risk), intermediate, and low affinity for MHCII, respectively. An IC₅₀ cutoff of 500 nM was used to evaluate HLA-DR alleles. IC₅₀ values >500 nM were not considered to have significant immunogenic potential.

The GAP residues located at the FVIII-XTEN junction of the chimeric protein was identified as having immunogenic potential. Applicant identified these GAP residues as encoded by a nucleotide sequence corresponding to a XhoI restriction enzyme site, originally introduced to facilitate cloning. The 9 nucleotides encoding the GAP residues were deleted from the coding sequence of the FVIII protein. Deletion of these nucleotides and the corresponding GAP residues in the translated protein was confirmed to eliminate the immunogenic potential at the FVIII-XTEN junction.

This resulted in the final FVIII nucleotide sequence encoding the chimeric FVIII protein tested herein, which was referred to as “coBDDFVIII6-XTEN-3aa”. The nucleotide sequence encoding coBDDFVIII6-XTEN-3aa is disclosed as SEQ ID NO: 11. The amino acid sequence of coBDDFVIII6-XTEN-3aa is disclosed as SEQ ID NO: 12 (see Table 1 for additional sequence information).

### Example 2: Generation of Genetic Expression Cassette Encoding coBDDFVIII-XTEN-3Aa

A genetic expression cassette was designed to carry the coBDDFVIII-XTEN-3aa transgene under the control of a hepatocyte-specific promoter for in vivo expression. The genetic expression cassette is flanked by 5′ and 3′ long terminal repeat (LTR) sequences that facilitate integration of the transfer plasmid sequence into the host genome.

LTR encoding elements include a chimeric 5′LTR fused to a heterologous human cytomegalovirus (CMV) early-gene promoter region (SEQ ID NO: 1), a self-inactivating (SIN) deletion of the enhancer/promoter sequence in the U3 region of the 3′ LTR (SEQ ID NO: X_annotated as “dU3RU5”), and a R region that allows for Tat binding and U5 region (SEQ ID NO: 2_RU5 region).

The transfer plasmid maintains the cis-acting viral sequences necessary for encapsidation, reverse transcription and integration in the host cell genome. The cis-acting viral sequences are the packaging signal (Psi, Y′), the primer binding site for SL123 (PBS) (SEQ ID NO: 3), the stem-loop 4 (SL4) (SEQ ID NO: 4), the polypurine tract (PPT) (SEQ ID NO: 6) required for reverse transcription, intron with donor and acceptor splice sites, and the Rev responsive element (RRE) (SEQ ID NO: 5) required for the Rev-mediated nuclear export of the unspliced, full genomic transcript. Additionally, the plasmid also encoded four tandem copies of the complementary sequence of hematopoietic-specific microRNA, miR-142-3pT (SEQ ID NO: 10), incorporated at the 3′ UTR to prevent transgene expression in hematopoietic-lineage antigen presenting cells while being maintained in non-hematopoietic cells (Brown et al. Nature 12:585-591 (2006).

The genetic cassette comprises a codon optimized cDNA encoding B-domain deleted human Factor VIII (BDDcoFVIII) fused with XTEN 144 peptide where three amino acid residues (Gly-Ala-Pro) at the FVIII/XTEN junction were removed to avoid potential MHCII-binding sites (XTEN-3aa) (see Example 1). This transgene, referred to as coBDDFVIII6-XTEN-3aa, is regulated by a liver-specific modified mouse transthyretin (mTTR) promoter (SEQ ID NO: 9) with two upstream enhancer sequences, mTTR enhancer element (SEQ ID NO: 8) and a synthetic enhancer (SEQ ID NO: 7).

A graphic representation of plasmid comprising the genetic expression cassette encoding coBDDFVIII6-XTEN-3aa is shown in FIG. 1.

The following examples test the functionality of the coBDDFVIII-XTEN-3aa transgene in vivo in multiple animal models.

### Example 3: Long-Term Dose Response of LV-coBDDFVIII6-XTEN-3Aa Treatment in HemA Neonatal Mice

To assess the efficacy of using a lentiviral system to express coBDDFVIII6-XTEN-3aa and produce FVIII activity in a pediatric HemA model, neonate (2-day-old) HemA mice were administered by temporal vein injection at about 1.5×10⁹, 3.0×10⁹, 6×10⁹, or 1.3×10¹⁰ TU/kg of LV-coBDDFVIII6-XTEN-3aa. Circulating FVIII activity was measured by FVIII chromogenic assay. Circulating FVIII protein was measured by human FVIII specific ELISA assay.

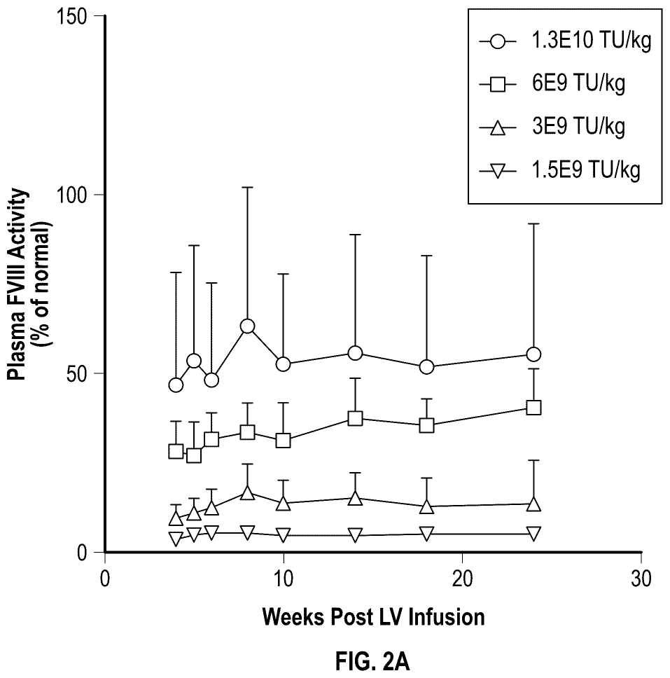

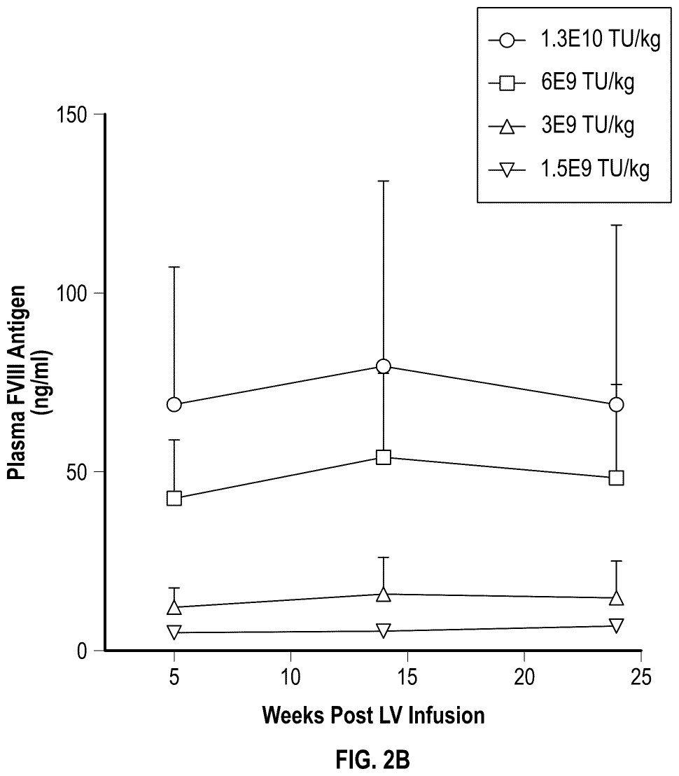

Persistent long-term FVIII expression was observed in a dose-dependent manner for all mice administered LV-coBDDFVIII6-XTEN-3aa. Post-lentiviral vector treatment, the FVIII activity levels for mice receiving LV-coBDDFVIII6-XTEN-3aa remained relatively stable through the end of the study at 25 weeks (FIG. 2A). The highest FVIII activity of about 50% was observed in mice receiving the 1.3×10¹⁰ TU/kg of LV-coBDDFVIII6-XTEN-3aa dose. Consistent with the FVIII activity data, levels of circulating FVIII protein remained relatively stable for all lentiviral doses through the end of the study (FIG. 2B).

These data demonstrate that coBDDFVIII6-XTEN-3aa delivered using a lentiviral system can produce therapeutic FVIII levels in neonatal HemA mice.

These data support the potential therapeutic benefit of using LV-coBDDFVIII6-XTEN-3aa to treat pediatric HemA patients.

### Example 4: Long-Term Dose Response of LV-coBDDFVIII6-XTEN-3Aa Treatment in HemA Adult Mice

To assess the efficacy of using a lentiviral system to express coBDDFVIII6-XTEN-3aa and produce FVIII activity in an adult HemA model, adult (16-week-old) HemA mice were administered by temporal vein injection at about 1.3×10¹⁰ or 3.7×10¹⁰ TU/kg of LV-coBDDFVIII6-XTEN-3aa. Circulating FVIII activity was measured by FVIII chromogenic assay. Circulating FVIII protein was measured by human FVIII specific ELISA assay.

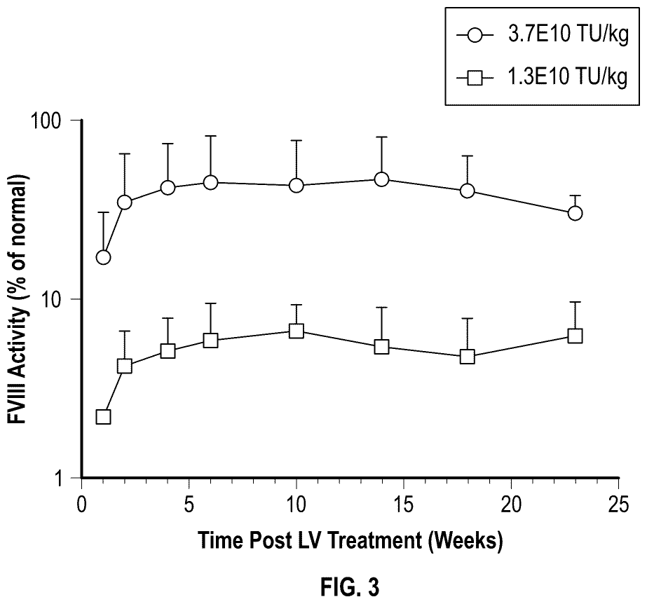

Persistent long-term FVIII expression was observed for both dosing groups in a dose-dependent manner. Post-lentiviral vector treatment, the FVIII activity levels for all mice receiving LV-coBDDFVIII6-XTEN-3aa remained relatively stable through the end of the study at at least 20 weeks (FIG. 3). Mice receiving 3.7×10¹⁰ TU/kg of LV-coBDDFVIII6-XTEN-3aa had a FVIII activity around 50% of normal for the duration of the study. Mice receiving the lower dose of 1.3×10¹⁰ TU/kg of LV-coBDDFVIII6-XTEN-3aa had a FVIII activity that was ≥5-7% of normal.

These data demonstrate that coBDDFVIII6-XTEN-3aa delivered using a lentiviral system can produce therapeutic FVIII levels in adult HemA mice.

These data support the potential therapeutic benefit of using LV-coBDDFVIII6-XTEN-3aa to treat adult HemA patients. These data also suggest that therapeutic benefits of using LV-coBDDFVIII6-XTEN-3aa may be achieved at relatively low doses of LV.

### Example 5: Long-Term Dose Response of LV-coBDDFVIII6-XTEN-3Aa Treatment in Non-Human Primates

To assess the efficacy of using a lentiviral system to express coBDDFVIII6-XTEN-3aa and produce FVIII activity in non-human primates, ten male pigtail macaques (3.5-4.3 kg body were treated with LV-coFVIII-6 or LV-coFVIII-6-XTEN produced from CD47high/MHC-I^{free} 293T cells via intravenous (IV) infusion at an infusion rate of 1.5 mL/minute. The dose for LV-coBDDFVIII6-XTEN-3aa was 1×10⁹ or 3×10⁹ TU/kg. To control anti-human FVIII antibody formation, animals were treated with daily intramuscular injection of SOLU-MEDROL® (methylprednisolone) from day-1 to day 7 of LV treatment at a dose of 10 mg/kg. Thirty (30) minutes before LV treatment, animals were also treated with IV injection of Polaraminc (dexchlorpheniramine) at a dose of 4 mg/kg to control potential allergic reactions.

Plasma samples were collected at 0, 1, 3, 7, 14, 21, 28, 45 and 60 days post-LV treatment and analyzed for human FVIII activity and FVIII antigen level. Circulating FVIII activity was measured by FVIII chromogenic assay. Circulating FVIII protein was measured by human FVIII specific ELISA assay. FVIII activity levels and FVIII antigen levels for each of the LV dosing groups were averaged across the post-treatment time points.

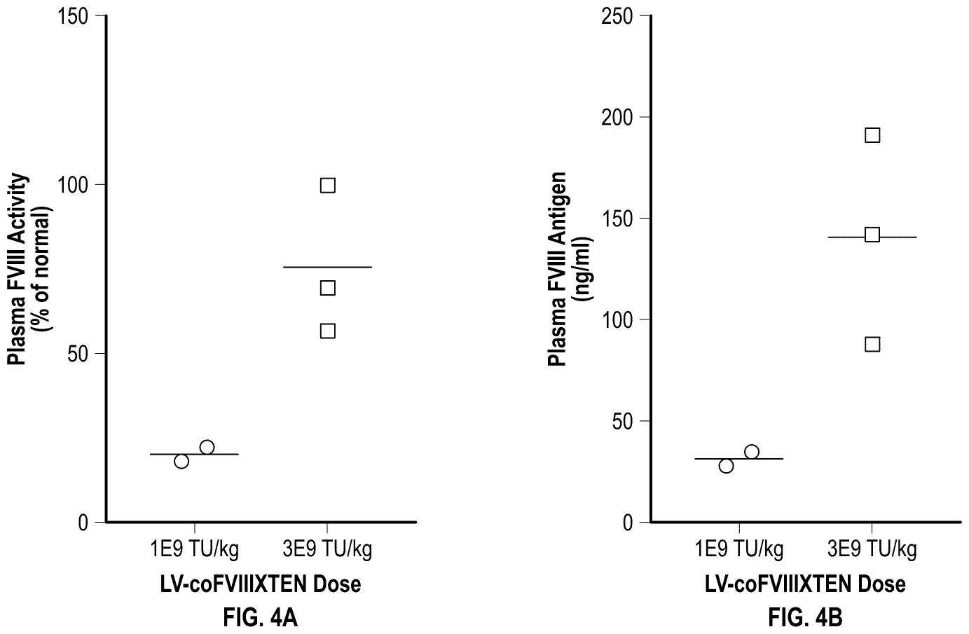

Average FVIII activity levels for the 1×10⁹ and 3×10⁹ TU/kg treatment group were about 20% and about 75% of normal, respectively (FIG. 4A). Average FVIII antigen levels for the 1×10⁹ or 3×10⁹ TU/kg treatment group were about 31 ng/ml or about 140 ng/ml, respectively (FIG. 4B).

These data demonstrate that LV-coBDDFVIII6-XTEN-3aa can produce therapeutic levels of human FVIII in non-human primates.

### SEQUENCES

TABLE 1
Nucleotide and Amino Acid Sequences

| SEQ ID NO/ | |
|---|---|
| Description | Nucleotide or amino acid sequence |
| SEQ ID | TGGCCATTGCATACGTTGTATCCATATCATAATATGTACATTTATATTG |
| NO. 1: | GCTCATGTCCAACATTACCGCCATGTTGACATTGATTATTGACTAGTTA |
| Human | TTAATAGTAATCAATTACGGGGTCATTAGTTCATAGCCCATATATGGA |
| CMV | GTTCCGCGTTACATAACTTACGGTAAATGGCCCGCCTGGCTGACCGCC |
| promoter | CAACGACCCCCGCCCATTGACGTCAATAATGACGTATGTTCCCATAGT |
| region- | AACGCCAATAGGGACTTTCCATTGACGTCAATGGGTGGAGTATTTACG |
| 5′LTR | GTAAACTGCCCACTTGGCAGTACATCAAGTGTATCATATGCCAAGTAC |
| | GCCCCCTATTGACGTCAATGACGGTAAATGGCCCGCCTGGCATTATGC |
| | CCAGTACATGACCTTATGGGACTTTCCTACTTGGCAGTACATCTACGT |
| | ATTAGTCATCGCTATTACCATGGTGATGCGGTTTTGGCAGTACATCAA |
| | TGGGCGTGGATAGCGGTTTGACTCACGGGGATTTCCAAGTCTCCACCC |
| | CATTGACGTCAATGGGAGTTTGTTTTGGCACCAAAATCAACGGGACTT |
| | TCCAAAATGTCGTAACAACTCCGCCCCATTGACGCAAATGGGCGGTAG |
| | GCGTGTACGGTGGGAGGTCTATATAAGCAGAGCTCGTTTAGTGAACC |
| SEQ ID | GGGTCTCTCTGGTTAGACCAGATCTGAGCCTGGGAGCTCTCTGGCTAA |
| NO. 2: | CTAGGGAACCCACTGCTTAAGCCTCAATAAAGCTTGCCTTGAGTGCTT |
| RU5 region | CAAGTAGTGTGTGCCCGTCTGTTGTGTGACTCTGGTAACTAGAGATCC |
| | CTCAGACCCTTTTAGTCAGTGTGGAAAATCTCTAGCAG |
| SEQ ID | TGGCGCCCGAACAGGGACCTGAAAGCGAAAGGGAAACCAGAGCTCTC |
| NO. 3: | TCGACGCAGGACTCGGCTTGCTGAAGCGCGCACGGCAAGAGGCGAGG |
| Primer | GGCGGCGACTGGTGAGTACGCCAAAAATTTTGACTAGCGGAGGCTAG |
| binding site | AAGGAGAGAG |
| (PBS) for | |
| SL123 | |
| SEQ ID | ATGGGTGCGAGAGCGTCAGTATTAAGCGGGGGAGAATTAGATCGCGA |
| NO. 4: | TGGGAAAAAATTCGGTTAAGGCCAGGGGGAAAGAAAAAATATAAATT |
| Stem-loop | AAAACATATAGTATGGGCAAGCAGGGAGCTAGAACGATTCGCAGTTA |
| 4 (SL4) | ATCCTGGCCTGTTAGAAACATCAGAAGGCTGTAGACAAATACTGGGA |
| mgag | CAGCTACAACCATCCCTTCAGACAGGATCAGAAGAACTTAGATCATTA |
| | TATAATACAGTAGCAACCCTCTATTGTGTGCATCAAAGGATAGAGATA |
| | AAAGACACCAAGGAAGCTTTAGACAAGATAGAGGAAGAGCAAAACA |
| | AAAGTAAGACCACCGCACAGCAAGCGGCCGCTGAT |
| SEQ ID | GGAGCTTTGTTCCTTGGGTTCTTGGGAGCAGCAGGAAGCACTATGGGC |
| NO. 5: | GCAGCCTCAATGACGCTGACGGTACAGGCCAGACAATTATTGTCTGGT |
| REV | ATAGTGCAGCAGCAGAACAATTTGCTGAGGGCTATTGAGGCGCAACA |
| Response | GCATCTGTTGCAACTCACAGTCTGGGGCATCAAGCAGCTCCAGGCAAG |
| Element | AATCCTGGCTGTGGAAAGATACCTAAAGGATCAACAGCTCCTGGGGA |
| (RRE) | TTT |
| SEQ ID | AACTTTTAAAAGAAAAGGGGGGATTGGGGGGTACAGTGCAGGGGAAA |
| NO. 6: | GAATAGTAGACATAATAGCAACAGACATACAAACTAAAGAATTACAA |
| Polypurine | AAACAAATTACAAAAATTCAAAATTTTATC |
| Tract (PPT) | |
| SEQ ID | CGCGAGTTAATAATTACCAGCGCGGGCCAAATAAATAATCCGCGAGG |
| NO. 7: | GGCAGGTGACGTTTGCCCAGCGCGCGCTGGTAATTATTAACCTCGCGA |
| Synthetic | ATATTGATTCGAGGCCGCGATTGCCGCAATCGCGAGGGGCAGGTGAC |
| enhancer | CTTTGCCCAGCGCGCG |
| SEQ ID | CACTGGGAGGATGTTGAGTAAGATGGAAAACTACTGATGACCCTTGC |
| NO. 8: | AGAGACAGAGTATTAGGACATGTTTGAACAGGGGCCGGGCGATCAGC |
| mTTR | AGGTAG |
| enhancer | |
| SEQ ID | GTCTGTCTGCACATTTCGTAGAGCGAGTGTTCCGATACTCTAATCTCCC |
| NO.9: | TAGGCAAGGTTCATATTTGTGTAGGTTACTTATTCTCCTTTTGTTGACT |
| mTTR | AAGTCAATAATCAGAATCAGCAGGTTTGGAGTCAGCTTGGCAGGGAT |
| promoter | CAGCAGCCTGGGTTGGAAGGAGGGGGTATAAAAGCCCCTTCACCAGG |
| | AGAAGCCGTC |
| SEQ ID | TCCATAAAGTAGGAAACACTACA |
| NO. 10: | |
| MicroRNA | |
| 142-3pT | |
| SEQ ID | ATGCAGATTGAGCTGTCCACTTGTTTCTTCCTGTGCCTCCTGCGCTTCT |
| NO. 11: | GTTTCTCCGCCACTCGCCGGTACTACCTTGGAGCCGTGGAGCTTTCAT |
| Nucleotide | GGGACTACATGCAGAGCGACCTGGGCGAACTCCCCGTGGATGCCAGA |
| sequence | TTCCCCCCCCGCGTGCCAAAGTCCTTCCCCTTTAACACCTCCGTGGTGT |
| encoding | ACAAGAAAACCCTCTTTGTCGAGTTCACTGACCACCTGTTCAACATCG |
| coBDDFVI | CCAAGCCGCGCCCACCTTGGATGGGCCTCCTGGGACCGACCATTCAAG |
| II6-XTEN- | CTGAAGTGTACGACACCGTGGTGATCACCCTGAAGAACATGGCGTCCC |
| 3aa | ACCCCGTGTCCCTGCATGCGGTCGGAGTGTCCTACTGGAAGGCCTCCG |
| | AAGGAGCTGAGTACGACGACCAGACTAGCCAGCGGGAAAAGGAGGA |
| | CGATAAAGTGTTCCCGGGCGGCTCGCATACTTACGTGTGGCAAGTCCT |
| | GAAGGAAAACGGACCTATGGCATCCGATCCTCTGTGCCTGACTTACTC |
| | CTACCTTTCCCATGTGGACCTCGTGAAGGACCTGAACAGCGGGCTGAT |
| | TGGTGCACTTCTCGTGTGCCGCGAAGGTTCGCTCGCTAAGGAAAAGAC |
| | CCAGACCCTCCATAAGTTCATCCTTTTGTTCGCTGTGTTCGATGAAGGA |
| | AAGTCATGGCATTCCGAAACTAAGAACTCGCTGATGCAGGACCGGGA |
| | TGCCGCCTCAGCCCGCGCCTGGCCTAAAATGCATACAGTCAACGGATA |
| | CGTGAATCGGTCACTGCCCGGGCTCATCGGTTGTCACAGAAAGTCCGT |
| | GTACTGGCACGTCATCGGCATGGGCACTACGCCTGAAGTGCACTCCAT |
| | CTTCCTGGAAGGGCACACCTTCCTCGTGCGCAACCACCGCCAGGCCTC |
| | TCTGGAAATCTCCCCGATTACCTTTCTGACCGCCCAGACTCTGCTCATG |
| | GACCTGGGGCAGTTCCTTCTCTTCTGCCACATCTCCAGCCATCAGCAC |
| | GACGGAATGGAGGCCTACGTGAAGGTGGACTCATGCCCGGAAGAACC |
| | TCAGTTGCGGATGAAGAACAACGAGGAGGCCGAGGACTATGACGACG |
| | ATTTGACTGACTCCGAGATGGACGTCGTGCGGTTCGATGACGACAACA |
| | GCCCCAGCTTCATCCAGATTCGCAGCGTGGCCAAGAAGCACCCCAAA |
| | ACCTGGGTGCACTACATCGCGGCCGAGGAAGAAGATTGGGACTACGC |
| | CCCGTTGGTGCTGGCACCCGATGACCGGTCGTACAAGTCCCAGTATCT |
| | GAACAATGGTCCGCAGCGGATTGGCAGAAAGTACAAGAAAGTGCGGT |
| | TCATGGCGTACACTGACGAAACGTTTAAGACCCGGGAGGCCATTCAA |
| | CATGAGAGCGGCATTCTGGGACCACTGCTGTACGGAGAGGTCGGCGA |
| | TACCCTGCTCATCATCTTCAAAAACCAGGCCTCCCGGCCTTACAACAT |
| | CTACCCTCACGGAATCACCGACGTGCGGCCACTCTACTCGCGGCGCCT |
| | GCCGAAGGGCGTCAAGCACCTGAAAGACTTCCCTATCCTGCCGGGCG |
| | AAATCTTCAAGTATAAGTGGACCGTCACCGTGGAGGACGGGCCCACC |
| | AAGAGCGATCCTAGGTGTCTGACTCGGTACTACTCCAGCTTCGTGAAC |
| | ATGGAACGGGACCTGGCATCGGGACTCATTGGACCGCTGCTGATCTGC |
| | TACAAAGAGTCGGTGGATCAACGCGGCAACCAGATCATGTCCGACAA |
| | GCGCAACGTGATCCTGTTCTCCGTGTTTGATGAAAACAGATCCTGGTA |
| | CCTCACTGAAAACATCCAGAGGTTCCTCCCAAACCCCGCAGGAGTGCA |
| | ACTGGAGGACCCTGAGTTTCAGGCCTCGAATATCATGCACTCGATTAA |
| | CGGTTACGTGTTCGACTCGCTGCAGCTGAGCGTGTGCCTCCATGAAGT |
| | CGCTTACTGGTACATTCTGTCCATCGGCGCCCAGACTGACTTCCTGAG |
| | CGTGTTCTTTTCCGGTTACACCTTTAAGCACAAGATGGTGTACGAAGA |
| | TACCCTGACCCTGTTCCCTTTCTCCGGCGAAACGGTGTTCATGTCGATG |
| | GAGAACCCGGGTCTGTGGATTCTGGGATGCCACAACAGCGACTTTCGG |
| | AACCGCGGAATGACTGCCCTGCTGAAGGTGTCCTCATGCGACAAGAA |
| | CACCGGAGACTACTACGAGGACTCCTACGAGGATATCTCAGCCTACCT |
| | CCTGTCCAAGAACAACGCGATCGAGCCGCGCAGCTTCAGCCAGAACA |
| | CATCAGAGAGCGCCACCCCTGAAAGTGGTCCCGGGAGCGAGCCAGCC |
| | ACATCTGGGTCGGAAACGCCAGGCACAAGTGAGTCTGCAACTCCCGA |
| | GTCCGGACCTGGCTCCGAGCCTGCCACTAGCGGCTCCGAGACTCCGGG |
| | AACTTCCGAGAGCGCTACACCAGAAAGCGGACCCGGAACCAGTACCG |
| | AACCTAGCGAGGGCTCTGCTCCGGGCAGCCCAGCCGGCTCTCCTACAT |
| | CCACGGAGGAGGGCACTTCCGAATCCGCCACCCCGGAGTCAGGGCCA |
| | GGATCTGAACCCGCTACCTCAGGCAGTGAGACGCCAGGAACGAGCGA |
| | GTCCGCTACACCGGAGAGTGGGCCAGGGAGCCCTGCTGGATCTCCTAC |
| | GTCCACTGAGGAAGGGTCACCAGCGGGCTCGCCCACCAGCACTGAAG |
| | AAGGTGCCTCGAGCCCGCCTGTGCTGAAGAGGCACCAGCGAGAAATT |
| | ACCCGGACCACCCTCCAATCGGATCAGGAGGAAATCGACTACGACGA |
| | CACCATCTCGGTGGAAATGAAGAAGGAAGATTTCGATATCTACGACG |
| | AGGACGAAAATCAGTCCCCTCGCTCATTCCAAAAGAAAACTAGACAC |
| | TACTTTATCGCCGCGGTGGAAAGACTGTGGGACTATGGAATGTCATCC |
| | AGCCCTCACGTCCTTCGGAACCGGGCCCAGAGCGGATCGGTGCCTCAG |
| | TTCAAGAAAGTGGTGTTCCAGGAGTTCACCGACGGCAGCTTCACCCAG |
| | CCGCTGTACCGGGGAGAACTGAACGAACACCTGGGCCTGCTCGGTCC |
| | CTACATCCGCGCGGAAGTGGAGGATAACATCATGGTGACCTTCCGTAA |
| | CCAAGCATCCAGACCTTACTCCTTCTATTCCTCCCTGATCTCATACGAG |
| | GAGGACCAGCGCCAAGGCGCCGAGCCCCGCAAGAACTTCGTCAAGCC |
| | CAACGAGACTAAGACCTACTTCTGGAAGGTCCAACACCATATGGCCCC |
| | GACCAAGGATGAGTTTGACTGCAAGGCCTGGGCCTACTTCTCCGACGT |
| | GGACCTTGAGAAGGATGTCCATTCCGGCCTGATCGGGCCGCTGCTCGT |
| | GTGTCACACCAACACCCTGAACCCAGCGCATGGACGCCAGGTCACCG |
| | TCCAGGAGTTTGCTCTGTTCTTCACCATTTTTGACGAAACTAAGTCCTG |
| | GTACTTCACCGAGAATATGGAGCGAAACTGTAGAGCGCCCTGCAATA |
| | TCCAGATGGAAGATCCGACTTTCAAGGAGAACTATAGATTCCACGCCA |
| | TCAACGGGTACATCATGGATACTCTGCCGGGGCTGGTCATGGCCCAGG |
| | ATCAGAGGATTCGGTGGTACTTGCTGTCAATGGGATCGAACGAAAAC |
| | ATTCACTCCATTCACTTCTCCGGTCACGTGTTCACTGTGCGCAAGAAG |
| | GAGGAGTACAAGATGGCGCTGTACAATCTGTACCCCGGGGTGTTCGA |
| | AACTGTGGAGATGCTGCCGTCCAAGGCCGGCATCTGGAGAGTGGAGT |
| | GCCTGATCGGAGAGCACCTCCACGCGGGGATGTCCACCCTCTTCCTGG |
| | TGTACTCGAATAAGTGCCAGACCCCGCTGGGCATGGCCTCGGGCCACA |
| | TCAGAGACTTCCAGATCACAGCAAGCGGACAATACGGCCAATGGGCG |
| | CCGAAGCTGGCCCGCTTGCACTACTCCGGATCGATCAACGCATGGTCC |
| | ACCAAGGAACCGTTCTCGTGGATTAAGGTGGACCTCCTGGCCCCTATG |
| | ATTATCCACGGAATTAAGACCCAGGGCGCCAGGCAGAAGTTCTCCTCC |
| | CTGTACATCTCGCAATTCATCATCATGTACAGCCTGGACGGGAAGAAG |
| | TGGCAGACTTACAGGGGAAACTCCACCGGCACCCTGATGGTCTTTTTC |
| | GGCAACGTGGATTCCTCCGGCATTAAGCACAACATCTTCAACCCACCG |
| | ATCATAGCCAGATATATTAGGCTCCACCCCACTCACTACTCAATCCGC |
| | TCAACTCTTCGGATGGAACTCATGGGGTGCGACCTGAACTCCTGCTCC |
| | ATGCCGTTGGGGATGGAATCAAAGGCTATTAGCGACGCCCAGATCAC |
| | CGCGAGCTCCTACTTCACTAACATGTTCGCCACCTGGAGCCCCTCCAA |
| | GGCCAGGCTGCACTTGCAGGGACGGTCAAATGCCTGGCGGCCGCAAG |
| | TGAACAATCCGAAGGAATGGCTTCAAGTGGATTTCCAAAAGACCATG |
| | AAAGTGACCGGAGTCACCACCCAGGGAGTGAAGTCCCTTCTGACCTC |
| | GATGTATGTGAAGGAGTTCCTGATTAGCAGCAGCCAGGACGGGCACC |
| | AGTGGACCCTGTTCTTCCAAAACGGAAAGGTCAAGGTGTTCCAGGGG |
| | AACCAGGACTCGTTCACACCCGTGGTGAACTCCCTGGACCCCCCACTG |
| | CTGACGCGGTACTTGAGGATTCATCCTCAGTCCTGGGTCCATCAGATT |
| | GCATTGCGAATGGAAGTCCTGGGCTGCGAGGCCCAGGACCTGTACTG |
| | A |
| SEQ ID | MQIELSTCFFLCLLRFCFSATRRYYLGAVELSWDYMQSDLGELPVDARFP |
| NO. 12: | PRVPKSFPFNTSVVYKKTLFVEFTDHLFNIAKPRPPWMGLLGPTIQAEVY |
| Amino | DTVVITLKNMASHPVSLHAVGVSYWKASEGAEYDDQTSQREKEDDKVF |
| Acid | PGGSHTYVWQVLKENGPMASDPLCLTYSYLSHVDLVKDLNSGLIGALLV |
| sequence of | CREGSLAKEKTQTLHKFILLFAVFDEGKSWHSETKNSLMQDRDAASARA |
| coBDDFVI | WPKMHTVNGYVNRSLPGLIGCHRKSVYWHVIGMGTTPEVHSIFLEGHTF |
| II6-XTEN- | LVRNHRQASLEISPITFLTAQTLLMDLGQFLLFCHISSHQHDGMEAYVKV |
| 3aa | DSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYK |
| | KVRFMAYTDETFKTREAIQHESGILGPLLYGEVGDTLLIIFKNQASRPYNI |
| | YPHGITDVRPLYSRRLPKGVKHLKDFPILPGEIFKYKWTVTVEDGPTKSDP |
| | RCLTRYYSSFVNMERDLASGLIGPLLICYKESVDQRGNQIMSDKRNVILFS |
| | VFDENRSWYLTENIQRFLPNPAGVQLEDPEFQASNIMHSINGYVFDSLQLS |
| | VCLHEVAYWYILSIGAQTDFLSVFFSGYTFKHKMVYEDTLTLFPFSGETV |
| | FMSMENPGLWILGCHNSDFRNRGMTALLKVSSCDKNTGDYYEDSYEDIS |
| | AYLLSKNNAIEPRSFSQNTSESATPESGPGSEPATSGSETPGTSESATPESG |
| | PGSEPATSGSETPGTSESATPESGPGTSTEPSEGSAPGSPAGSPTSTEEGTSE |
| | SATPESGPGSEPATSGSETPGTSESATPESGPGSPAGSPTSTEEGSPAGSPTS |
| | TEEGASSPPVLKRHQREITRTTLQSDQEEIDYDDTISVEMKKEDFDIYDED |
| | ENQSPRSFQKKTRHYFIAAVERLWDYGMSSSPHVLRNRAQSGSVPQFKK |
| | VVFQEFTDGSFTQPLYRGELNEHLGLLGPYIRAEVEDNIMVTFRNQASRP |
| | YSFYSSLISYEEDQRQGAEPRKNFVKPNETKTYFWKVQHHMAPTKDEFD |
| | CKAWAYFSDVDLEKDVHSGLIGPLLVCHTNTLNPAHGRQVTVQEFALFF |
| | TIFDETKSWYFTENMERNCRAPCNIQMEDPTFKENYRFHAINGYIMDTLP |
| | GLVMAQDQRIRWYLLSMGSNENIHSIHFSGHVFTVRKKEEYKMALYNLY |
| | PGVFETVEMLPSKAGIWRVECLIGEHLHAGMSTLFLVYSNKCQTPLGMA |
| | SGHIRDFQITASGQYGQWAPKLARLHYSGSINAWSTKEPFSWIKVDLLAP |
| | MIIHGIKTQGARQKFSSLYISQFIIMYSLDGKKWQTYRGNSTGTLMVFFGN |
| | VDSSGIKHNIFNPPIIARYIRLHPTHYSIRSTLRMELMGCDLNSCSMPLGME |
| | SKAISDAQITASSYFTNMFATWSPSKARLHLQGRSNAWRPQVNNPKEWL |
| | QVDFQKTMKVTGVTTQGVKSLLTSMYVKEFLISSSQDGHQWTLFFQNGK |
| | VKVFQGNQDSFTPVVNSLDPPLLTRYLRIHPQSWVHQIALRMEVLGCEA |
| | QDLY- |
| SEQ ID | MQIELSTCFFLCLLRFCFS |
| NO. 13: | |
| Signal | |
| peptide of | |
| coBDDFVI | |
| II6-XTEN- | |
| 3aa | |
| SEQ ID | ATGCAGATTGAGCTGTCCACTTGTTTCTTCCTGTGCCTCCTGCGCTTCT |
| NO. 14: | GTTTCTCCGCCACTCGCCGGTACTACCTTGGAGCCGTGGAGCTTTCAT |
| Nucleotide | GGGACTACATGCAGAGCGACCTGGGCGAACTCCCCGTGGATGCCAGA |
| sequence | TTCCCCCCCCGCGTGCCAAAGTCCTTCCCCTTTAACACCTCCGTGGTGT |
| encoding | ACAAGAAAACCCTCTTTGTCGAGTTCACTGACCACCTGTTCAACATCG |
| coBDDFVI | CCAAGCCGCGCCCACCTTGGATGGGCCTCCTGGGACCGACCATTCAAG |
| II6-3aa | CTGAAGTGTACGACACCGTGGTGATCACCCTGAAGAACATGGCGTCCC |
| | ACCCCGTGTCCCTGCATGCGGTCGGAGTGTCCTACTGGAAGGCCTCCG |
| | AAGGAGCTGAGTACGACGACCAGACTAGCCAGCGGGAAAAGGAGGA |
| | CGATAAAGTGTTCCCGGGCGGCTCGCATACTTACGTGTGGCAAGTCCT |
| | GAAGGAAAACGGACCTATGGCATCCGATCCTCTGTGCCTGACTTACTC |
| | CTACCTTTCCCATGTGGACCTCGTGAAGGACCTGAACAGCGGGCTGAT |
| | TGGTGCACTTCTCGTGTGCCGCGAAGGTTCGCTCGCTAAGGAAAAGAC |
| | CCAGACCCTCCATAAGTTCATCCTTTTGTTCGCTGTGTTCGATGAAGGA |
| | AAGTCATGGCATTCCGAAACTAAGAACTCGCTGATGCAGGACCGGGA |
| | TGCCGCCTCAGCCCGCGCCTGGCCTAAAATGCATACAGTCAACGGATA |
| | CGTGAATCGGTCACTGCCCGGGCTCATCGGTTGTCACAGAAAGTCCGT |
| | GTACTGGCACGTCATCGGCATGGGCACTACGCCTGAAGTGCACTCCAT |
| | CTTCCTGGAAGGGCACACCTTCCTCGTGCGCAACCACCGCCAGGCCTC |
| | TCTGGAAATCTCCCCGATTACCTTTCTGACCGCCCAGACTCTGCTCATG |
| | GACCTGGGGCAGTTCCTTCTCTTCTGCCACATCTCCAGCCATCAGCAC |
| | GACGGAATGGAGGCCTACGTGAAGGTGGACTCATGCCCGGAAGAACC |
| | TCAGTTGCGGATGAAGAACAACGAGGAGGCCGAGGACTATGACGACG |
| | ATTTGACTGACTCCGAGATGGACGTCGTGCGGTTCGATGACGACAACA |
| | GCCCCAGCTTCATCCAGATTCGCAGCGTGGCCAAGAAGCACCCCAAA |
| | ACCTGGGTGCACTACATCGCGGCCGAGGAAGAAGATTGGGACTACGC |
| | CCCGTTGGTGCTGGCACCCGATGACCGGTCGTACAAGTCCCAGTATCT |
| | GAACAATGGTCCGCAGCGGATTGGCAGAAAGTACAAGAAAGTGCGGT |
| | TCATGGCGTACACTGACGAAACGTTTAAGACCCGGGAGGCCATTCAA |
| | CATGAGAGCGGCATTCTGGGACCACTGCTGTACGGAGAGGTCGGCGA |
| | TACCCTGCTCATCATCTTCAAAAACCAGGCCTCCCGGCCTTACAACAT |
| | CTACCCTCACGGAATCACCGACGTGCGGCCACTCTACTCGCGGCGCCT |
| | GCCGAAGGGCGTCAAGCACCTGAAAGACTTCCCTATCCTGCCGGGCG |
| | AAATCTTCAAGTATAAGTGGACCGTCACCGTGGAGGACGGGCCCACC |
| | AAGAGCGATCCTAGGTGTCTGACTCGGTACTACTCCAGCTTCGTGAAC |
| | ATGGAACGGGACCTGGCATCGGGACTCATTGGACCGCTGCTGATCTGC |
| | TACAAAGAGTCGGTGGATCAACGCGGCAACCAGATCATGTCCGACAA |
| | GCGCAACGTGATCCTGTTCTCCGTGTTTGATGAAAACAGATCCTGGTA |
| | CCTCACTGAAAACATCCAGAGGTTCCTCCCAAACCCCGCAGGAGTGCA |
| | ACTGGAGGACCCTGAGTTTCAGGCCTCGAATATCATGCACTCGATTAA |
| | CGGTTACGTGTTCGACTCGCTGCAGCTGAGCGTGTGCCTCCATGAAGT |
| | CGCTTACTGGTACATTCTGTCCATCGGCGCCCAGACTGACTTCCTGAG |
| | CGTGTTCTTTTCCGGTTACACCTTTAAGCACAAGATGGTGTACGAAGA |
| | TACCCTGACCCTGTTCCCTTTCTCCGGCGAAACGGTGTTCATGTCGATG |
| | GAGAACCCGGGTCTGTGGATTCTGGGATGCCACAACAGCGACTTTCGG |
| | AACCGCGGAATGACTGCCCTGCTGAAGGTGTCCTCATGCGACAAGAA |
| | CACCGGAGACTACTACGAGGACTCCTACGAGGATATCTCAGCCTACCT |
| | CCTGTCCAAGAACAACGCGATCGAGCCGCGCAGCTTCAGCCAGAACC |
| | CGCCTGTGCTGAAGAGGCACCAGCGAGAAATTACCCGGACCACCCTC |
| | CAATCGGATCAGGAGGAAATCGACTACGACGACACCATCTCGGTGGA |
| | AATGAAGAAGGAAGATTTCGATATCTACGACGAGGACGAAAATCAGT |
| | CCCCTCGCTCATTCCAAAAGAAAACTAGACACTACTTTATCGCCGCGG |
| | TGGAAAGACTGTGGGACTATGGAATGTCATCCAGCCCTCACGTCCTTC |
| | GGAACCGGGCCCAGAGCGGATCGGTGCCTCAGTTCAAGAAAGTGGTG |
| | TTCCAGGAGTTCACCGACGGCAGCTTCACCCAGCCGCTGTACCGGGGA |
| | GAACTGAACGAACACCTGGGCCTGCTCGGTCCCTACATCCGCGCGGA |
| | AGTGGAGGATAACATCATGGTGACCTTCCGTAACCAAGCATCCAGAC |
| | CTTACTCCTTCTATTCCTCCCTGATCTCATACGAGGAGGACCAGCGCC |
| | AAGGCGCCGAGCCCCGCAAGAACTTCGTCAAGCCCAACGAGACTAAG |
| | ACCTACTTCTGGAAGGTCCAACACCATATGGCCCCGACCAAGGATGA |
| | GTTTGACTGCAAGGCCTGGGCCTACTTCTCCGACGTGGACCTTGAGAA |
| | GGATGTCCATTCCGGCCTGATCGGGCCGCTGCTCGTGTGTCACACCAA |
| | CACCCTGAACCCAGCGCATGGACGCCAGGTCACCGTCCAGGAGTTTGC |
| | TCTGTTCTTCACCATTTTTGACGAAACTAAGTCCTGGTACTTCACCGAG |
| | AATATGGAGCGAAACTGTAGAGCGCCCTGCAATATCCAGATGGAAGA |
| | TCCGACTTTCAAGGAGAACTATAGATTCCACGCCATCAACGGGTACAT |
| | CATGGATACTCTGCCGGGGCTGGTCATGGCCCAGGATCAGAGGATTCG |
| | GTGGTACTTGCTGTCAATGGGATCGAACGAAAACATTCACTCCATTCA |
| | CTTCTCCGGTCACGTGTTCACTGTGCGCAAGAAGGAGGAGTACAAGAT |
| | GGCGCTGTACAATCTGTACCCCGGGGTGTTCGAAACTGTGGAGATGCT |
| | GCCGTCCAAGGCCGGCATCTGGAGAGTGGAGTGCCTGATCGGAGAGC |
| | ACCTCCACGCGGGGATGTCCACCCTCTTCCTGGTGTACTCGAATAAGT |
| | GCCAGACCCCGCTGGGCATGGCCTCGGGCCACATCAGAGACTTCCAG |
| | ATCACAGCAAGCGGACAATACGGCCAATGGGCGCCGAAGCTGGCCCG |
| | CTTGCACTACTCCGGATCGATCAACGCATGGTCCACCAAGGAACCGTT |
| | CTCGTGGATTAAGGTGGACCTCCTGGCCCCTATGATTATCCACGGAAT |
| | TAAGACCCAGGGCGCCAGGCAGAAGTTCTCCTCCCTGTACATCTCGCA |
| | ATTCATCATCATGTACAGCCTGGACGGGAAGAAGTGGCAGACTTACA |
| | GGGGAAACTCCACCGGCACCCTGATGGTCTTTTTCGGCAACGTGGATT |
| | CCTCCGGCATTAAGCACAACATCTTCAACCCACCGATCATAGCCAGAT |
| | ATATTAGGCTCCACCCCACTCACTACTCAATCCGCTCAACTCTTCGGAT |
| | GGAACTCATGGGGTGCGACCTGAACTCCTGCTCCATGCCGTTGGGGAT |
| | GGAATCAAAGGCTATTAGCGACGCCCAGATCACCGCGAGCTCCTACTT |
| | CACTAACATGTTCGCCACCTGGAGCCCCTCCAAGGCCAGGCTGCACTT |
| | GCAGGGACGGTCAAATGCCTGGCGGCCGCAAGTGAACAATCCGAAGG |
| | AATGGCTTCAAGTGGATTTCCAAAAGACCATGAAAGTGACCGGAGTC |
| | ACCACCCAGGGAGTGAAGTCCCTTCTGACCTCGATGTATGTGAAGGAG |
| | TTCCTGATTAGCAGCAGCCAGGACGGGCACCAGTGGACCCTGTTCTTC |
| | CAAAACGGAAAGGTCAAGGTGTTCCAGGGGAACCAGGACTCGTTCAC |
| | ACCCGTGGTGAACTCCCTGGACCCCCCACTGCTGACGCGGTACTTGAG |
| | GATTCATCCTCAGTCCTGGGTCCATCAGATTGCATTGCGAATGGAAGT |
| | CCTGGGCTGCGAGGCCCAGGACCTGTACTGA |
| SEQ ID | MQIELSTCFFLCLLRFCFSATRRYYLGAVELSWDYMQSDLGELPVDARFP |
| NO. 15: | PRVPKSFPFNTSVVYKKTLFVEFTDHLFNIAKPRPPWMGLLGPTIQAEVY |
| Amino acid | DTVVITLKNMASHPVSLHAVGVSYWKASEGAEYDDQTSQREKEDDKVF |
| sequence of | PGGSHTYVWQVLKENGPMASDPLCLTYSYLSHVDLVKDLNSGLIGALLV |
| coBDDFVI | CREGSLAKEKTQTLHKFILLFAVFDEGKSWHSETKNSLMQDRDAASARA |
| II-3aa | WPKMHTVNGYVNRSLPGLIGCHRKSVYWHVIGMGTTPEVHSIFLEGHTF |
| | LVRNHRQASLEISPITFLTAQTLLMDLGQFLLFCHISSHQHDGMEAYVKV |
| | DSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYK |
| | KVRFMAYTDETFKTREAIQHESGILGPLLYGEVGDTLLIIFKNQASRPYNI |
| | YPHGITDVRPLYSRRLPKGVKHLKDFPILPGEIFKYKWTVTVEDGPTKSDP |
| | RCLTRYYSSFVNMERDLASGLIGPLLICYKESVDQRGNQIMSDKRNVILFS |
| | VFDENRSWYLTENIQRFLPNPAGVQLEDPEFQASNIMHSINGYVFDSLQLS |
| | VCLHEVAYWYILSIGAQTDFLSVFFSGYTFKHKMVYEDTLTLFPFSGETV |
| | FMSMENPGLWILGCHNSDFRNRGMTALLKVSSCDKNTGDYYEDSYEDIS |
| | AYLLSKNNAIEPRSFSQNPPVLKRHQREITRTTLQSDQEEIDYDDTISVEM |
| | KKEDFDIYDEDENQSPRSFQKKTRHYFIAAVERLWDYGMSSSPHVLRNR |
| | AQSGSVPQFKKVVFQEFTDGSFTQPLYRGELNEHLGLLGPYIRAEVEDNI |
| | MVTFRNQASRPYSFYSSLISYEEDQRQGAEPRKNFVKPNETKTYFWKVQ |
| | HHMAPTKDEFDCKAWAYFSDVDLEKDVHSGLIGPLLVCHTNTLNPAHG |
| | RQVTVQEFALFFTIFDETKSWYFTENMERNCRAPCNIQMEDPTFKENYRF |
| | HAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVY |
| | SNKCQTPLGMASGHIRDFQITASGQYGQWAPKLARLHYSGSINAWSTKE |
| | PFSWIKVDLLAPMIIHGIKTQGARQKFSSLYISQFIIMYSLDGKKWQTYRG |
| | NSTGTLMVFFGNVDSSGIKHNIFNPPIIARYIRLHPTHYSIRSTLRMELMGC |
| | DLNSCSMPLGMESKAISDAQITASSYFTNMFATWSPSKARLHLQGRSNA |
| | WRPQVNNPKEWLQVDFQKTMKVTGVTTQGVKSLLTSMYVKEFLISSSQ |
| | DGHQWTLFFQNGKVKVFQGNQDSFTPVVNSLDPPLLTRYLRIHPQSWVH |
| | QIALRMEVLGCEAQDLY- |
| SEQ ID | TGGCCATTGCATACGTTGTATCCATATCATAATATGTACATTTATATTG |
| NO: 16 | GCTCATGTCCAACATTACCGCCATGTTGACATTGATTATTGACTAGTTA |
| Nucleotide | TTAATAGTAATCAATTACGGGGTCATTAGTTCATAGCCCATATATGGA |
| sequence of | GTTCCGCGTTACATAACTTACGGTAAATGGCCCGCCTGGCTGACCGCC |
| genetic | CAACGACCCCCGCCCATTGACGTCAATAATGACGTATGTTCCCATAGT |
| expression | AACGCCAATAGGGACTTTCCATTGACGTCAATGGGTGGAGTATTTACG |
| cassette for | GTAAACTGCCCACTTGGCAGTACATCAAGTGTATCATATGCCAAGTAC |
| coBDDFVI | GCCCCCTATTGACGTCAATGACGGTAAATGGCCCGCCTGGCATTATGC |
| II6-XTEN- | CCAGTACATGACCTTATGGGACTTTCCTACTTGGCAGTACATCTACGT |
| 3aa | ATTAGTCATCGCTATTACCATGGTGATGCGGTTTTGGCAGTACATCAA |
| | TGGGCGTGGATAGCGGTTTGACTCACGGGGATTTCCAAGTCTCCACCC |
| | CATTGACGTCAATGGGAGTTTGTTTTGGCACCAAAATCAACGGGACTT |
| | TCCAAAATGTCGTAACAACTCCGCCCCATTGACGCAAATGGGCGGTAG |
| | GCGTGTACGGTGGGAGGTCTATATAAGCAGAGCTCGTTTAGTGAACCG |
| | GGGTCTCTCTGGTTAGACCAGATCTGAGCCTGGGAGCTCTCTGGCTAA |
| | CTAGGGAACCCACTGCTTAAGCCTCAATAAAGCTTGCCTTGAGTGCTT |
| | CAAGTAGTGTGTGCCCGTCTGTTGTGTGACTCTGGTAACTAGAGATCC |
| | CTCAGACCCTTTTAGTCAGTGTGGAAAATCTCTAGCAGTGGCGCCCGA |
| | ACAGGGACCTGAAAGCGAAAGGGAAACCAGAGCTCTCTCGACGCAGG |
| | ACTCGGCTTGCTGAAGCGCGCACGGCAAGAGGCGAGGGGCGGCGACT |
| | GGTGAGTACGCCAAAAATTTTGACTAGCGGAGGCTAGAAGGAGAGAG |
| | ATGGGTGCGAGAGCGTCAGTATTAAGCGGGGGAGAATTAGATCGCGA |
| | TGGGAAAAAATTCGGTTAAGGCCAGGGGGAAAGAAAAAATATAAATT |
| | AAAACATATAGTATGGGCAAGCAGGGAGCTAGAACGATTCGCAGTTA |
| | ATCCTGGCCTGTTAGAAACATCAGAAGGCTGTAGACAAATACTGGGA |
| | CAGCTACAACCATCCCTTCAGACAGGATCAGAAGAACTTAGATCATTA |
| | TATAATACAGTAGCAACCCTCTATTGTGTGCATCAAAGGATAGAGATA |
| | AAAGACACCAAGGAAGCTTTAGACAAGATAGAGGAAGAGCAAAACA |
| | AAAGTAAGACCACCGCACAGCAAGCGGCCGCTGATCTTCAGACCTGG |
| | AGGAGGAGATATGAGGGACAATTGGAGAAGTGAATTATATAAATATA |
| | AAGTAGTAAAAATTGAACCATTAGGAGTAGCACCCACCAAGGCAAAG |
| | AGAAGAGTGGTGCAGAGAGAAAAAAGAGCAGTGGGAATAGGAGCTT |
| | TGTTCCTTGGGTTCTTGGGAGCAGCAGGAAGCACTATGGGCGCAGCCT |
| | CAATGACGCTGACGGTACAGGCCAGACAATTATTGTCTGGTATAGTGC |
| | AGCAGCAGAACAATTTGCTGAGGGCTATTGAGGCGCAACAGCATCTG |
| | TTGCAACTCACAGTCTGGGGCATCAAGCAGCTCCAGGCAAGAATCCTG |
| | GCTGTGGAAAGATACCTAAAGGATCAACAGCTCCTGGGGATTTGGGG |
| | TTGCTCTGGAAAACTCATTTGCACCACTGCTGTGCCTTGGAATGCTAG |
| | TTGGAGTAATAAATCTCTGGAACAGATTTGGAATCACACGACCTGGAT |
| | GGAGTGGGACAGAGAAATTAACAATTACACAAGCTTAATACACTCCT |
| | TAATTGAAGAATCGCAAAACCAGCAAGAAAAGAATGAACAAGAATTA |
| | TTGGAATTAGATAAATGGGCAAGTTTGTGGAATTGGTTTAACATAACA |
| | AATTGGCTGTGGTATATAAAATTATTCATAATGATAGTAGGAGGCTTG |
| | GTAGGTTTAAGAATAGTTTTTGCTGTACTTTCTATAGTGAATAGAGTTA |
| | GGCAGGGATATTCACCATTATCGTTTCAGACCCACCTCCCAACCCCGA |
| | GGGGACCCGACAGGCCCGAAGGAATAGAAGAAGAAGGTGGAGAGAG |
| | AGACAGAGACAGATCCATTCGATTAGTGAACGGATCTCGACGGTATC |
| | GGTTAACTTTTAAAAGAAAAGGGGGGATTGGGGGGTACAGTGCAGGG |
| | GAAAGAATAGTAGACATAATAGCAACAGACATACAAACTAAAGAATT |
| | ACAAAAACAAATTACAAAAATTCAAAATTTTATCGATCACGAGACTA |
| | GCCTCGAGCACGCGAGTTAATAATTACCAGCGCGGGCCAAATAAATA |
| | ATCCGCGAGGGGCAGGTGACGTTTGCCCAGCGCGCGCTGGTAATTATT |
| | AACCTCGCGAATATTGATTCGAGGCCGCGATTGCCGCAATCGCGAGG |
| | GGCAGGTGACCTTTGCCCAGCGCGCGTTCGCCCCGCCCCGGACGGTAT |
| | CGATAAGCTTAGGAGCTTGGGCTGCAGGTCGAGGGCACTGGGAGGAT |
| | GTTGAGTAAGATGGAAAACTACTGATGACCCTTGCAGAGACAGAGTA |
| | TTAGGACATGTTTGAACAGGGGCCGGGCGATCAGCAGGTAGCTCTAG |
| | AGGATCCCCGTCTGTCTGCACATTTCGTAGAGCGAGTGTTCCGATACT |
| | CTAATCTCCCTAGGCAAGGTTCATATTTGTGTAGGTTACTTATTCTCCT |
| | TTTGTTGACTAAGTCAATAATCAGAATCAGCAGGTTTGGAGTCAGCTT |
| | GGCAGGGATCAGCAGCCTGGGTTGGAAGGAGGGGGTATAAAAGCCCC |
| | TTCACCAGGAGAAGCCGTCACACAGATCCACAAGCTCCTGGCTAGCG |
| | CCACCATGCAGATTGAGCTGTCCACTTGTTTCTTCCTGTGCCTCCTGCG |
| | CTTCTGTTTCTCCGCCACTCGCCGGTACTACCTTGGAGCCGTGGAGCTT |
| | TCATGGGACTACATGCAGAGCGACCTGGGCGAACTCCCCGTGGATGC |
| | CAGATTCCCCCCCCGCGTGCCAAAGTCCTTCCCCTTTAACACCTCCGTG |
| | GTGTACAAGAAAACCCTCTTTGTCGAGTTCACTGACCACCTGTTCAAC |
| | ATCGCCAAGCCGCGCCCACCTTGGATGGGCCTCCTGGGACCGACCATT |
| | CAAGCTGAAGTGTACGACACCGTGGTGATCACCCTGAAGAACATGGC |
| | GTCCCACCCCGTGTCCCTGCATGCGGTCGGAGTGTCCTACTGGAAGGC |
| | CTCCGAAGGAGCTGAGTACGACGACCAGACTAGCCAGCGGGAAAAGG |
| | AGGACGATAAAGTGTTCCCGGGCGGCTCGCATACTTACGTGTGGCAA |
| | GTCCTGAAGGAAAACGGACCTATGGCATCCGATCCTCTGTGCCTGACT |
| | TACTCCTACCTTTCCCATGTGGACCTCGTGAAGGACCTGAACAGCGGG |
| | CTGATTGGTGCACTTCTCGTGTGCCGCGAAGGTTCGCTCGCTAAGGAA |
| | AAGACCCAGACCCTCCATAAGTTCATCCTTTTGTTCGCTGTGTTCGATG |
| | AAGGAAAGTCATGGCATTCCGAAACTAAGAACTCGCTGATGCAGGAC |
| | CGGGATGCCGCCTCAGCCCGCGCCTGGCCTAAAATGCATACAGTCAAC |
| | GGATACGTGAATCGGTCACTGCCCGGGCTCATCGGTTGTCACAGAAAG |
| | TCCGTGTACTGGCACGTCATCGGCATGGGCACTACGCCTGAAGTGCAC |
| | TCCATCTTCCTGGAAGGGCACACCTTCCTCGTGCGCAACCACCGCCAG |
| | GCCTCTCTGGAAATCTCCCCGATTACCTTTCTGACCGCCCAGACTCTGC |
| | TCATGGACCTGGGGCAGTTCCTTCTCTTCTGCCACATCTCCAGCCATCA |
| | GCACGACGGAATGGAGGCCTACGTGAAGGTGGACTCATGCCCGGAAG |
| | AACCTCAGTTGCGGATGAAGAACAACGAGGAGGCCGAGGACTATGAC |
| | GACGATTTGACTGACTCCGAGATGGACGTCGTGCGGTTCGATGACGAC |
| | AACAGCCCCAGCTTCATCCAGATTCGCAGCGTGGCCAAGAAGCACCC |
| | CAAAACCTGGGTGCACTACATCGCGGCCGAGGAAGAAGATTGGGACT |
| | ACGCCCCGTTGGTGCTGGCACCCGATGACCGGTCGTACAAGTCCCAGT |
| | ATCTGAACAATGGTCCGCAGCGGATTGGCAGAAAGTACAAGAAAGTG |
| | CGGTTCATGGCGTACACTGACGAAACGTTTAAGACCCGGGAGGCCATT |
| | CAACATGAGAGCGGCATTCTGGGACCACTGCTGTACGGAGAGGTCGG |
| | CGATACCCTGCTCATCATCTTCAAAAACCAGGCCTCCCGGCCTTACAA |
| | CATCTACCCTCACGGAATCACCGACGTGCGGCCACTCTACTCGCGGCG |
| | CCTGCCGAAGGGCGTCAAGCACCTGAAAGACTTCCCTATCCTGCCGGG |
| | CGAAATCTTCAAGTATAAGTGGACCGTCACCGTGGAGGACGGGCCCA |
| | CCAAGAGCGATCCTAGGTGTCTGACTCGGTACTACTCCAGCTTCGTGA |
| | ACATGGAACGGGACCTGGCATCGGGACTCATTGGACCGCTGCTGATCT |
| | GCTACAAAGAGTCGGTGGATCAACGCGGCAACCAGATCATGTCCGAC |
| | AAGCGCAACGTGATCCTGTTCTCCGTGTTTGATGAAAACAGATCCTGG |
| | TACCTCACTGAAAACATCCAGAGGTTCCTCCCAAACCCCGCAGGAGTG |
| | CAACTGGAGGACCCTGAGTTTCAGGCCTCGAATATCATGCACTCGATT |
| | AACGGTTACGTGTTCGACTCGCTGCAGCTGAGCGTGTGCCTCCATGAA |
| | GTCGCTTACTGGTACATTCTGTCCATCGGCGCCCAGACTGACTTCCTG |
| | AGCGTGTTCTTTTCCGGTTACACCTTTAAGCACAAGATGGTGTACGAA |
| | GATACCCTGACCCTGTTCCCTTTCTCCGGCGAAACGGTGTTCATGTCG |
| | ATGGAGAACCCGGGTCTGTGGATTCTGGGATGCCACAACAGCGACTTT |
| | CGGAACCGCGGAATGACTGCCCTGCTGAAGGTGTCCTCATGCGACAA |
| | GAACACCGGAGACTACTACGAGGACTCCTACGAGGATATCTCAGCCT |
| | ACCTCCTGTCCAAGAACAACGCGATCGAGCCGCGCAGCTTCAGCCAG |
| | AACACATCAGAGAGCGCCACCCCTGAAAGTGGTCCCGGGAGCGAGCC |
| | AGCCACATCTGGGTCGGAAACGCCAGGCACAAGTGAGTCTGCAACTC |
| | CCGAGTCCGGACCTGGCTCCGAGCCTGCCACTAGCGGCTCCGAGACTC |
| | CGGGAACTTCCGAGAGCGCTACACCAGAAAGCGGACCCGGAACCAGT |
| | ACCGAACCTAGCGAGGGCTCTGCTCCGGGCAGCCCAGCCGGCTCTCCT |
| | ACATCCACGGAGGAGGGCACTTCCGAATCCGCCACCCCGGAGTCAGG |
| | GCCAGGATCTGAACCCGCTACCTCAGGCAGTGAGACGCCAGGAACGA |
| | GCGAGTCCGCTACACCGGAGAGTGGGCCAGGGAGCCCTGCTGGATCT |
| | CCTACGTCCACTGAGGAAGGGTCACCAGCGGGCTCGCCCACCAGCAC |
| | TGAAGAAGGTGCCTCGAGCCCGCCTGTGCTGAAGAGGCACCAGCGAG |
| | AAATTACCCGGACCACCCTCCAATCGGATCAGGAGGAAATCGACTAC |
| | GACGACACCATCTCGGTGGAAATGAAGAAGGAAGATTTCGATATCTA |
| | CGACGAGGACGAAAATCAGTCCCCTCGCTCATTCCAAAAGAAAACTA |
| | GACACTACTTTATCGCCGCGGTGGAAAGACTGTGGGACTATGGAATGT |
| | CATCCAGCCCTCACGTCCTTCGGAACCGGGCCCAGAGCGGATCGGTGC |
| | CTCAGTTCAAGAAAGTGGTGTTCCAGGAGTTCACCGACGGCAGCTTCA |
| | CCCAGCCGCTGTACCGGGGAGAACTGAACGAACACCTGGGCCTGCTC |
| | GGTCCCTACATCCGCGCGGAAGTGGAGGATAACATCATGGTGACCTTC |
| | CGTAACCAAGCATCCAGACCTTACTCCTTCTATTCCTCCCTGATCTCAT |
| | ACGAGGAGGACCAGCGCCAAGGCGCCGAGCCCCGCAAGAACTTCGTC |
| | AAGCCCAACGAGACTAAGACCTACTTCTGGAAGGTCCAACACCATAT |
| | GGCCCCGACCAAGGATGAGTTTGACTGCAAGGCCTGGGCCTACTTCTC |
| | CGACGTGGACCTTGAGAAGGATGTCCATTCCGGCCTGATCGGGCCGCT |
| | GCTCGTGTGTCACACCAACACCCTGAACCCAGCGCATGGACGCCAGGT |
| | CACCGTCCAGGAGTTTGCTCTGTTCTTCACCATTTTTGACGAAACTAAG |
| | TCCTGGTACTTCACCGAGAATATGGAGCGAAACTGTAGAGCGCCCTGC |
| | AATATCCAGATGGAAGATCCGACTTTCAAGGAGAACTATAGATTCCAC |
| | GCCATCAACGGGTACATCATGGATACTCTGCCGGGGCTGGTCATGGCC |
| | CAGGATCAGAGGATTCGGTGGTACTTGCTGTCAATGGGATCGAACGA |
| | AAACATTCACTCCATTCACTTCTCCGGTCACGTGTTCACTGTGCGCAA |
| | GAAGGAGGAGTACAAGATGGCGCTGTACAATCTGTACCCCGGGGTGT |
| | TCGAAACTGTGGAGATGCTGCCGTCCAAGGCCGGCATCTGGAGAGTG |
| | GAGTGCCTGATCGGAGAGCACCTCCACGCGGGGATGTCCACCCTCTTC |
| | CTGGTGTACTCGAATAAGTGCCAGACCCCGCTGGGCATGGCCTCGGGC |
| | CACATCAGAGACTTCCAGATCACAGCAAGCGGACAATACGGCCAATG |
| | GGCGCCGAAGCTGGCCCGCTTGCACTACTCCGGATCGATCAACGCATG |
| | GTCCACCAAGGAACCGTTCTCGTGGATTAAGGTGGACCTCCTGGCCCC |
| | TATGATTATCCACGGAATTAAGACCCAGGGCGCCAGGCAGAAGTTCTC |
| | CTCCCTGTACATCTCGCAATTCATCATCATGTACAGCCTGGACGGGAA |
| | GAAGTGGCAGACTTACAGGGGAAACTCCACCGGCACCCTGATGGTCT |
| | TTTTCGGCAACGTGGATTCCTCCGGCATTAAGCACAACATCTTCAACC |
| | CACCGATCATAGCCAGATATATTAGGCTCCACCCCACTCACTACTCAA |
| | TCCGCTCAACTCTTCGGATGGAACTCATGGGGTGCGACCTGAACTCCT |
| | GCTCCATGCCGTTGGGGATGGAATCAAAGGCTATTAGCGACGCCCAG |
| | ATCACCGCGAGCTCCTACTTCACTAACATGTTCGCCACCTGGAGCCCC |
| | TCCAAGGCCAGGCTGCACTTGCAGGGACGGTCAAATGCCTGGCGGCC |
| | GCAAGTGAACAATCCGAAGGAATGGCTTCAAGTGGATTTCCAAAAGA |
| | CCATGAAAGTGACCGGAGTCACCACCCAGGGAGTGAAGTCCCTTCTG |
| | ACCTCGATGTATGTGAAGGAGTTCCTGATTAGCAGCAGCCAGGACGG |
| | GCACCAGTGGACCCTGTTCTTCCAAAACGGAAAGGTCAAGGTGTTCCA |
| | GGGGAACCAGGACTCGTTCACACCCGTGGTGAACTCCCTGGACCCCCC |
| | ACTGCTGACGCGGTACTTGAGGATTCATCCTCAGTCCTGGGTCCATCA |
| | GATTGCATTGCGAATGGAAGTCCTGGGCTGCGAGGCCCAGGACCTGT |
| | ACTGA |
| SEQ ID | ATRRYYLGAVELSWDYMQSDLGELPVDARFPPRVPKSFPFNTSVVYKKT |
| NO: 17 | LFVEFTDHLFNIAKPRPPWMGLLGPTIQAEVYDTVVITLKNMASHPVSLH |
| Amino acid | AVGVSYWKASEGAEYDDQTSQREKEDDKVFPGGSHTYVWQVLKENGP |
| sequence of | MASDPLCLTYSYLSHVDLVKDLNSGLIGALLVCREGSLAKEKTQTLHKFI |
| BDD | LLFAVFDEGKSWHSETKNSLMQDRDAASARAWPKMHTVNGYVNRSLPG |
| mature | LIGCHRKSVYWHVIGMGTTPEVHSIFLEGHTFLVRNHRQASLEISPITFLTA |
| human | QTLLMDLGQFLLFCHISSHQHDGMEAYVKVDSCPEEPQLRMKNNEEAED |
| FVIII | YDDDLTDSEMDVVRFDDDNSPSFIQIRSVAKKHPKTWVHYIAAEEEDWD |
| | YAPLVLAPDDRSYKSQYLNNGPQRIGRKYKKVRFMAYTDETFKTREAIQ |
| | HESGILGPLLYGEVGDTLLIIFKNQASRPYNIYPHGITDVRPLYSRRLPKGV |
| | KHLKDFPILPGEIFKYKWTVTVEDGPTKSDPRCLTRYYSSFVNMERDLAS |
| | GLIGPLLICYKESVDQRGNQIMSDKRNVILFSVFDENRSWYLTENIQRFLP |
| | NPAGVQLEDPEFQASNIMHSINGYVFDSLQLSVCLHEVAYWYILSIGAQT |
| | DFLSVFFSGYTFKHKMVYEDTLTLFPFSGETVFMSMENPGLWILGCHNSD |
| | FRNRGMTALLKVSSCDKNTGDYYEDSYEDISAYLLSKNNAIEPRSFSQNP |
| | PVLKRHQREITRTTLQSDQEEIDYDDTISVEMKKEDFDIYDEDENQSPRSF |
| | QKKTRHYFIAAVERLWDYGMSSSPHVLRNRAQSGSVPQFKKVVFQEFTD |
| | GSFTQPLYRGELNEHLGLLGPYIRAEVEDNIMVTFRNQASRPYSFYSSLIS |
| | YEEDQRQGAEPRKNFVKPNETKTYFWKVQHHMAPTKDEFDCKAWAYFS |
| | DVDLEKDVHSGLIGPLLVCHTNTLNPAHGRQVTVQEFALFFTIFDETKSW |
| | YFTENMERNCRAPCNIQMEDPTFKENYRFHAINGYIMDTLPGLVMAQDQ |
| | RIRWYLLSMGSNENIHSIHFSGHVFTVRKKEEYKMALYNLYPGVFETVE |
| | MLPSKAGIWRVECLIGEHLHAGMSTLFLVYSNKCQTPLGMASGHIRDFQI |
| | TASGQYGQWAPKLARLHYSGSINAWSTKEPFSWIKVDLLAPMIIHGIKTQ |
| | GARQKFSSLYISQFIIMYSLDGKKWQTYRGNSTGTLMVFFGNVDSSGIKH |
| | NIFNPPIIARYIRLHPTHYSIRSTLRMELMGCDLNSCSMPLGMESKAISDAQ |
| | ITASSYFTNMFATWSPSKARLHLQGRSNAWRPQVNNPKEWLQVDFQKT |
| | MKVTGVTTQGVKSLLTSMYVKEFLISSSQDGHQWTLFFQNGKVKVFQG |
| | NQDSFTPVVNSLDPPLLTRYLRIHPQSWVHQIALRMEVLGCEAQDLY |
| SEQ ID | ATGCAAATAGAGCTCTCCACCTGCTTCTTTCTGTGCCTTTTGCGATTCT |
| NO: 18 | GCTTTAGTGCCACCAGAAGATACTACCTGGGTGCAGTGGAACTGTCAT |
| Nucleic | GGGACTATATGCAAAGTGATCTCGGTGAGCTGCCTGTGGACGCAAGA |
| acid | TTTCCTCCTAGAGTGCCAAAATCTTTTCCATTCAACACCTCAGTCGTGT |
| sequence | ACAAAAAGACTCTGTTTGTAGAATTCACGGATCACCTTTTCAACATCG |
| encoding | CTAAGCCAAGGCCACCCTGGATGGGTCTGCTAGGTCCTACCATCCAGG |
| wild type | CTGAGGTTTATGATACAGTGGTCATTACACTTAAGAACATGGCTTCCC |
| human | ATCCTGTCAGTCTTCATGCTGTTGGTGTATCCTACTGGAAAGCTTCTGA |
| FVIII | GGGAGCTGAATATGATGATCAGACCAGTCAAAGGGAGAAAGAAGATG |
| | ATAAAGTCTTCCCTGGTGGAAGCCATACATATGTCTGGCAGGTCCTGA |
| | AAGAGAATGGTCCAATGGCCTCTGACCCACTGTGCCTTACCTACTCAT |
| | ATCTTTCTCATGTGGACCTGGTAAAAGACTTGAATTCAGGCCTCATTG |
| | GAGCCCTACTAGTATGTAGAGAAGGGAGTCTGGCCAAGGAAAAGACA |
| | CAGACCTTGCACAAATTTATACTACTTTTTGCTGTATTTGATGAAGGG |
| | AAAAGTTGGCACTCAGAAACAAAGAACTCCTTGATGCAGGATAGGGA |
| | TGCTGCATCTGCTCGGGCCTGGCCTAAAATGCACACAGTCAATGGTTA |
| | TGTAAACAGGTCTCTGCCAGGTCTGATTGGATGCCACAGGAAATCAGT |
| | CTATTGGCATGTGATTGGAATGGGCACCACTCCTGAAGTGCACTCAAT |
| | ATTCCTCGAAGGTCACACATTTCTTGTGAGGAACCATCGCCAGGCGTC |
| | CTTGGAAATCTCGCCAATAACTTTCCTTACTGCTCAAACACTCTTGATG |
| | GACCTTGGACAGTTTCTACTGTTTTGTCATATCTCTTCCCACCAACATG |
| | ATGGCATGGAAGCTTATGTCAAAGTAGACAGCTGTCCAGAGGAACCC |
| | CAACTACGAATGAAAAATAATGAAGAAGCGGAAGACTATGATGATGA |
| | TCTTACTGATTCTGAAATGGATGTGGTCAGGTTTGATGATGACAACTC |
| | TCCTTCCTTTATCCAAATTCGCTCAGTTGCCAAGAAGCATCCTAAAACT |
| | TGGGTACATTACATTGCTGCTGAAGAGGAGGACTGGGACTATGCTCCC |
| | TTAGTCCTCGCCCCCGATGACAGAAGTTATAAAAGTCAATATTTGAAC |
| | AATGGCCCTCAGCGGATTGGTAGGAAGTACAAAAAAGTCCGATTTAT |
| | GGCATACACAGATGAAACCTTTAAGACTCGTGAAGCTATTCAGCATGA |
| | ATCAGGAATCTTGGGACCTTTACTTTATGGGGAAGTTGGAGACACACT |
| | GTTGATTATATTTAAGAATCAAGCAAGCAGACCATATAACATCTACCC |
| | TCACGGAATCACTGATGTCCGTCCTTTGTATTCAAGGAGATTACCAAA |
| | AGGTGTAAAACATTTGAAGGATTTTCCAATTCTGCCAGGAGAAATATT |
| | CAAATATAAATGGACAGTGACTGTAGAAGATGGGCCAACTAAATCAG |
| | ATCCTCGGTGCCTGACCCGCTATTACTCTAGTTTCGTTAATATGGAGA |
| | GAGATCTAGCTTCAGGACTCATTGGCCCTCTCCTCATCTGCTACAAAG |
| | AATCTGTAGATCAAAGAGGAAACCAGATAATGTCAGACAAGAGGAAT |
| | GTCATCCTGTTTTCTGTATTTGATGAGAACCGAAGCTGGTACCTCACA |
| | GAGAATATACAACGCTTTCTCCCCAATCCAGCTGGAGTGCAGCTTGAG |
| | GATCCAGAGTTCCAAGCCTCCAACATCATGCACAGCATCAATGGCTAT |
| | GTTTTTGATAGTTTGCAGTTGTCAGTTTGTTTGCATGAGGTGGCATACT |
| | GGTACATTCTAAGCATTGGAGCACAGACTGACTTCCTTTCTGTCTTCTT |
| | CTCTGGATATACCTTCAAACACAAAATGGTCTATGAAGACACACTCAC |
| | CCTATTCCCATTCTCAGGAGAAACTGTCTTCATGTCGATGGAAAACCC |
| | AGGTCTATGGATTCTGGGGTGCCACAACTCAGACTTTCGGAACAGAGG |
| | CATGACCGCCTTACTGAAGGTTTCTAGTTGTGACAAGAACACTGGTGA |
| | TTATTACGAGGACAGTTATGAAGATATTTCAGCATACTTGCTGAGTAA |
| | AAACAATGCCATTGAACCAAGAAGCTTCTCCCAGAATTCAAGACACC |
| | CTAGCACTAGGCAAAAGCAATTTAATGCCACCACAATTCCAGAAAAT |
| | GACATAGAGAAGACTGACCCTTGGTTTGCACACAGAACACCTATGCCT |
| | AAAATACAAAATGTCTCCTCTAGTGATTTGTTGATGCTCTTGCGACAG |
| | AGTCCTACTCCACATGGGCTATCCTTATCTGATCTCCAAGAAGCCAAA |
| | TATGAGACTTTTTCTGATGATCCATCACCTGGAGCAATAGACAGTAAT |
| | AACAGCCTGTCTGAAATGACACACTTCAGGCCACAGCTCCATCACAGT |
| | GGGGACATGGTATTTACCCCTGAGTCAGGCCTCCAATTAAGATTAAAT |
| | GAGAAACTGGGGACAACTGCAGCAACAGAGTTGAAGAAACTTGATTT |
| | CAAAGTTTCTAGTACATCAAATAATCTGATTTCAACAATTCCATCAGA |
| | CAATTTGGCAGCAGGTACTGATAATACAAGTTCCTTAGGACCCCCAAG |
| | TATGCCAGTTCATTATGATAGTCAATTAGATACCACTCTATTTGGCAA |
| | AAAGTCATCTCCCCTTACTGAGTCTGGTGGACCTCTGAGCTTGAGTGA |
| | AGAAAATAATGATTCAAAGTTGTTAGAATCAGGTTTAATGAATAGCCA |
| | AGAAAGTTCATGGGGAAAAAATGTATCGTCAACAGAGAGTGGTAGGT |
| | TATTTAAAGGGAAAAGAGCTCATGGACCTGCTTTGTTGACTAAAGATA |
| | ATGCCTTATTCAAAGTTAGCATCTCTTTGTTAAAGACAAACAAAACTT |
| | CCAATAATTCAGCAACTAATAGAAAGACTCACATTGATGGCCCATCAT |
| | TATTAATTGAGAATAGTCCATCAGTCTGGCAAAATATATTAGAAAGTG |
| | ACACTGAGTTTAAAAAAGTGACACCTTTGATTCATGACAGAATGCTTA |
| | TGGACAAAAATGCTACAGCTTTGAGGCTAAATCATATGTCAAATAAA |
| | ACTACTTCATCAAAAAACATGGAAATGGTCCAACAGAAAAAAGAGGG |
| | CCCCATTCCACCAGATGCACAAAATCCAGATATGTCGTTCTTTAAGAT |
| | GCTATTCTTGCCAGAATCAGCAAGGTGGATACAAAGGACTCATGGAA |
| | AGAACTCTCTGAACTCTGGGCAAGGCCCCAGTCCAAAGCAATTAGTAT |
| | CCTTAGGACCAGAAAAATCTGTGGAAGGTCAGAATTTCTTGTCTGAGA |
| | AAAACAAAGTGGTAGTAGGAAAGGGTGAATTTACAAAGGACGTAGGA |
| | CTCAAAGAGATGGTTTTTCCAAGCAGCAGAAACCTATTTCTTACTAAC |
| | TTGGATAATTTACATGAAAATAATACACACAATCAAGAAAAAAAAAT |
| | TCAGGAAGAAATAGAAAAGAAGGAAACATTAATCCAAGAGAATGTA |
| | GTTTTGCCTCAGATACATACAGTGACTGGCACTAAGAATTTCATGAAG |
| | AACCTTTTCTTACTGAGCACTAGGCAAAATGTAGAAGGTTCATATGAC |
| | GGGGCATATGCTCCAGTACTTCAAGATTTTAGGTCATTAAATGATTCA |
| | ACAAATAGAACAAAGAAACACACAGCTCATTTCTCAAAAAAAGGGGA |
| | GGAAGAAAACTTGGAAGGCTTGGGAAATCAAACCAAGCAAATTGTAG |
| | AGAAATATGCATGCACCACAAGGATATCTCCTAATACAAGCCAGCAG |
| | AATTTTGTCACGCAACGTAGTAAGAGAGCTTTGAAACAATTCAGACTC |
| | CCACTAGAAGAAACAGAACTTGAAAAAAGGATAATTGTGGATGACAC |
| | CTCAACCCAGTGGTCCAAAAACATGAAACATTTGACCCCGAGCACCCT |
| | CACACAGATAGACTACAATGAGAAGGAGAAAGGGGCCATTACTCAGT |
| | CTCCCTTATCAGATTGCCTTACGAGGAGTCATAGCATCCCTCAAGCAA |
| | ATAGATCTCCATTACCCATTGCAAAGGTATCATCATTTCCATCTATTAG |
| | ACCTATATATCTGACCAGGGTCCTATTCCAAGACAACTCTTCTCATCTT |
| | CCAGCAGCATCTTATAGAAAGAAAGATTCTGGGGTCCAAGAAAGCAG |
| | TCATTTCTTACAAGGAGCCAAAAAAAATAACCTTTCTTTAGCCATTCT |
| | AACCTTGGAGATGACTGGTGATCAAAGAGAGGTTGGCTCCCTGGGGA |
| | CAAGTGCCACAAATTCAGTCACATACAAGAAAGTTGAGAACACTGTT |
| | CTCCCGAAACCAGACTTGCCCAAAACATCTGGCAAAGTTGAATTGCTT |
| | CCAAAAGTTCACATTTATCAGAAGGACCTATTCCCTACGGAAACTAGC |
| | AATGGGTCTCCTGGCCATCTGGATCTCGTGGAAGGGAGCCTTCTTCAG |
| | GGAACAGAGGGAGCGATTAAGTGGAATGAAGCAAACAGACCTGGAA |
| | AAGTTCCCTTTCTGAGAGTAGCAACAGAAAGCTCTGCAAAGACTCCCT |
| | CCAAGCTATTGGATCCTCTTGCTTGGGATAACCACTATGGTACTCAGA |
| | TACCAAAAGAAGAGTGGAAATCCCAAGAGAAGTCACCAGAAAAAAC |
| | AGCTTTTAAGAAAAAGGATACCATTTTGTCCCTGAACGCTTGTGAAAG |
| | CAATCATGCAATAGCAGCAATAAATGAGGGACAAAATAAGCCCGAAA |
| | TAGAAGTCACCTGGGCAAAGCAAGGTAGGACTGAAAGGCTGTGCTCT |
| | CAAAACCCACCAGTCTTGAAACGCCATCAACGGGAAATAACTCGTAC |
| | TACTCTTCAGTCAGATCAAGAGGAAATTGACTATGATGATACCATATC |
| | AGTTGAAATGAAGAAGGAAGATTTTGACATTTATGATGAGGATGAAA |
| | ATCAGAGCCCCCGCAGCTTTCAAAAGAAAACACGACACTATTTTATTG |
| | CTGCAGTGGAGAGGCTCTGGGATTATGGGATGAGTAGCTCCCCACATG |
| | TTCTAAGAAACAGGGCTCAGAGTGGCAGTGTCCCTCAGTTCAAGAAA |
| | GTTGTTTTCCAGGAATTTACTGATGGCTCCTTTACTCAGCCCTTATACC |
| | GTGGAGAACTAAATGAACATTTGGGACTCCTGGGGCCATATATAAGA |
| | GCAGAAGTTGAAGATAATATCATGGTAACTTTCAGAAATCAGGCCTCT |
| | CGTCCCTATTCCTTCTATTCTAGCCTTATTTCTTATGAGGAAGATCAGA |
| | GGCAAGGAGCAGAACCTAGAAAAAACTTTGTCAAGCCTAATGAAACC |
| | AAAACTTACTTTTGGAAAGTGCAACATCATATGGCACCCACTAAAGAT |
| | GAGTTTGACTGCAAAGCCTGGGCTTATTTCTCTGATGTTGACCTGGAA |
| | AAAGATGTGCACTCAGGCCTGATTGGACCCCTTCTGGTCTGCCACACT |
| | AACACACTGAACCCTGCTCATGGGAGACAAGTGACAGTACAGGAATT |
| | TGCTCTGTTTTTCACCATCTTTGATGAGACCAAAAGCTGGTACTTCACT |
| | GAAAATATGGAAAGAAACTGCAGGGCTCCCTGCAATATCCAGATGGA |
| | AGATCCCACTTTTAAAGAGAATTATCGCTTCCATGCAATCAATGGCTA |
| | CATAATGGATACACTACCTGGCTTAGTAATGGCTCAGGATCAAAGGAT |
| | TCGATGGTATCTGCTCAGCATGGGCAGCAATGAAAACATCCATTCTAT |
| | TCATTTCAGTGGACATGTGTTCACTGTACGAAAAAAAGAGGAGTATAA |
| | AATGGCACTGTACAATCTCTATCCAGGTGTTTTTGAGACAGTGGAAAT |
| | GTTACCATCCAAAGCTGGAATTTGGCGGGTGGAATGCCTTATTGGCGA |
| | GCATCTACATGCTGGGATGAGCACACTTTTTCTGGTGTACAGCAATAA |
| | GTGTCAGACTCCCCTGGGAATGGCTTCTGGACACATTAGAGATTTTCA |
| | GATTACAGCTTCAGGACAATATGGACAGTGGGCCCCAAAGCTGGCCA |
| | GACTTCATTATTCCGGATCAATCAATGCCTGGAGCACCAAGGAGCCCT |
| | TTTCTTGGATCAAGGTGGATCTGTTGGCACCAATGATTATTCACGGCA |
| | TCAAGACCCAGGGTGCCCGTCAGAAGTTCTCCAGCCTCTACATCTCTC |
| | AGTTTATCATCATGTATAGTCTTGATGGGAAGAAGTGGCAGACTTATC |
| | GAGGAAATTCCACTGGAACCTTAATGGTCTTCTTTGGCAATGTGGATT |
| | CATCTGGGATAAAACACAATATTTTTAACCCTCCAATTATTGCTCGAT |
| | ACATCCGTTTGCACCCAACTCATTATAGCATTCGCAGCACTCTTCGCAT |
| | GGAGTTGATGGGCTGTGATTTAAATAGTTGCAGCATGCCATTGGGAAT |
| | GGAGAGTAAAGCAATATCAGATGCACAGATTACTGCTTCATCCTACTT |
| | TACCAATATGTTTGCCACCTGGTCTCCTTCAAAAGCTCGACTTCACCTC |
| | CAAGGGAGGAGTAATGCCTGGAGACCTCAGGTGAATAATCCAAAAGA |
| | GTGGCTGCAAGTGGACTTCCAGAAGACAATGAAAGTCACAGGAGTAA |
| | CTACTCAGGGAGTAAAATCTCTGCTTACCAGCATGTATGTGAAGGAGT |
| | TCCTCATCTCCAGCAGTCAAGATGGCCATCAGTGGACTCTCTTTTTTCA |
| | GAATGGCAAAGTAAAGGTTTTTCAGGGAAATCAAGACTCCTTCACACC |
| | TGTGGTGAACTCTCTAGACCCACCGTTACTGACTCGCTACCTTCGAATT |
| | CACCCCCAGAGTTGGGTGCACCAGATTGCCCTGAGGATGGAGGTTCTG |
| | GGCTGCGAGGCACAGGACCTCTAC |
| SEQ ID | ATRRYYLGAVELSWDYMQSDLGELPVDARFPPRVPKSFPFNTSVVYKKT |
| NO: 19 | LFVEFTDHLFNIAKPRPPWMGLLGPTIQAEVYDTVVITLKNMASHPVSLH |
| Amino acid | AVGVSYWKASEGAEYDDQTSQREKEDDKVFPGGSHTYVWQVLKENGP |
| sequence of | MASDPLCLTYSYLSHVDLVKDLNSGLIGALLVCREGSLAKEKTQTLHKFI |
| wild type | LLFAVFDEGKSWHSETKNSLMQDRDAASARAWPKMHTVNGYVNRSLPG |
| human | LIGCHRKSVYWHVIGMGTTPEVHSIFLEGHTFLVRNHRQASLEISPITFLTA |
| mature | QTLLMDLGQFLLFCHISSHQHDGMEAYVKVDSCPEEPQLRMKNNEEAED |
| FVIII | YDDDLTDSEMDVVRFDDDNSPSFIQIRSVAKKHPKTWVHYIAAEEEDWD |
| protein | YAPLVLAPDDRSYKSQYLNNGPQRIGRKYKKVRFMAYTDETFKTREAIQ |
| | HESGILGPLLYGEVGDTLLIIFKNQASRPYNIYPHGITDVRPLYSRRLPKGV |
| | KHLKDFPILPGEIFKYKWTVTVEDGPTKSDPRCLTRYYSSFVNMERDLAS |
| | GLIGPLLICYKESVDQRGNQIMSDKRNVILFSVFDENRSWYLTENIQRFLP |
| | NPAGVQLEDPEFQASNIMHSINGYVFDSLQLSVCLHEVAYWYILSIGAQT |
| | DFLSVFFSGYTFKHKMVYEDTLTLFPFSGETVFMSMENPGLWILGCHNSD |
| | FRNRGMTALLKVSSCDKNTGDYYEDSYEDISAYLLSKNNAIEPRSFSQNS |
| | RHPSTRQKQFNATTIPENDIEKTDPWFAHRTPMPKIQNVSSSDLLMLLRQS |
| | PTPHGLSLSDLQEAKYETFSDDPSPGAIDSNNSLSEMTHFRPQLHHSGDM |
| | VFTPESGLQLRLNEKLGTTAATELKKLDFKVSSTSNNLISTIPSDNLAAGT |
| | DNTSSLGPPSMPVHYDSQLDTTLFGKKSSPLTESGGPLSLSEENNDSKLLE |
| | SGLMNSQESSWGKNVSSTESGRLFKGKRAHGPALLTKDNALFKVSISLLK |
| | TNKTSNNSATNRKTHIDGPSLLIENSPSVWQNILESDTEFKKVTPLIHDRM |
| | LMDKNATALRLNHMSNKTTSSKNMEMVQQKKEGPIPPDAQNPDMSFFK |
| | MLFLPESARWIQRTHGKNSLNSGQGPSPKQLVSLGPEKSVEGQNFLSEKN |
| | KVVVGKGEFTKDVGLKEMVFPSSRNLFLTNLDNLHENNTHNQEKKIQEE |
| | IEKKETLIQENVVLPQIHTVTGTKNFMKNLFLLSTRQNVEGSYDGAYAPV |
| | LQDFRSLNDSTNRTKKHTAHFSKKGEEENLEGLGNQTKQIVEKYACTTRI |
| | SPNTSQQNFVTQRSKRALKQFRLPLEETELEKRIIVDDTSTQWSKNMKHL |
| | TPSTLTQIDYNEKEKGAITQSPLSDCLTRSHSIPQANRSPLPIAKVSSFPSIRP |
| | IYLTRVLFQDNSSHLPAASYRKKDSGVQESSHFLQGAKKNNLSLAILTLE |
| | MTGDQREVGSLGTSATNSVTYKKVENTVLPKPDLPKTSGKVELLPKVHI |
| | YQKDLFPTETSNGSPGHLDLVEGSLLQGTEGAIKWNEANRPGKVPFLRV |
| | ATESSAKTPSKLLDPLAWDNHYGTQIPKEEWKSQEKSPEKTAFKKKDTIL |
| | SLNACESNHAIAAINEGQNKPEIEVTWAKQGRTERLCSQNPPVLKRHQRE |
| | ITRTTLQSDQEEIDYDDTISVEMKKEDFDIYDEDENQSPRSFQKKTRHYFI |
| | AAVERLWDYGMSSSPHVLRNRAQSGSVPQFKKVVFQEFTDGSFTQPLYR |
| | GELNEHLGLLGPYIRAEVEDNIMVTFRNQASRPYSFYSSLISYEEDQRQGA |
| | EPRKNFVKPNETKTYFWKVQHHMAPTKDEFDCKAWAYFSDVDLEKDV |
| | HSGLIGPLLVCHTNTLNPAHGRQVTVQEFALFFTIFDETKSWYFTENMER |
| | NCRAPCNIQMEDPTFKENYRFHAINGYIMDTLPGLVMAQDQRIRWYLLS |
| | MGSNENIHSIHFSGHVFTVRKKEEYKMALYNLYPGVFETVEMLPSKAGI |
| | WRVECLIGEHLHAGMSTLFLVYSNKCQTPLGMASGHIRDFQITASGQYG |
| | QWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIA |
| | RYIRLHPTHYSIRSTLRMELMGCDLNSCSMPLGMESKAISDAQITASSYFT |
| | NMFATWSPSKARLHLQGRSNAWRPQVNNPKEWLQVDFQKTMKVTGVT |
| | TQGVKSLLTSMYVKEFLISSSQDGHQWTLFFQNGKVKVFQGNQDSFTPV |
| | VNSLDPPLLTRYLRIHPQSWVHQIALRMEVLGCEAQDLY |

1. An isolated nucleic acid molecule comprising a nucleotide sequence having at least 85% sequence identity to SEQ ID NO:11, wherein the nucleotide sequence encodes a polypeptide with factor VIII (FVIII) activity.

2. The isolated nucleic acid molecule of claim 1, wherein the nucleotide sequence has at least 90%, at least 95%, at least 96%, at least 97%, at least 98%, at least 99%, or 100% sequence identity to SEQ ID NO: 11.

3.-4. (canceled)

5. The isolated nucleic acid molecule of claim 1, wherein the nucleotide sequence comprises a nucleotide sequence having at least 90%, at least 95%, at least 96%, at least 97%, at least 98%, at least 99%, or 100% sequence identity to nucleotides 58-4815 of SEQ ID NO: 11.

6. (canceled)

7. An isolated nucleic acid molecule comprising a nucleotide sequence having at least 85% sequence identity to SEQ ID NO: 14 or SEQ ID NO: 16, wherein the nucleotide sequence encodes a polypeptide with factor VIII (FVIII) activity.

8. The isolated nucleic acid molecule of claim 7, wherein the nucleotide sequence is at least 90% sequence identity, at least 95%, at least 96%, at least 97%, at least 98%, at least 99%, or 100% sequence identity to SEQ ID NO: 14.

9.-11. (canceled)

12. The isolated nucleic acid molecule of claim 7, wherein the genetic cassette comprises a nucleotide sequence having at least 90%, at least 95%, at least 96%, at least 97%, at least 98%, at least 99%, or 100% sequence identity to SEQ ID NO: 16.

13.-14. (canceled)

15. An isolated nucleic acid molecule of claim 1, comprising a genetic cassette expressing a Factor VIII (FVIII) polypeptide comprising:
i) a nucleotide sequence encoding a FVIII protein comprising a nucleic acid sequence having at least 85% sequence identity to SEQ ID NO: 11;
ii) a promoter controlling transcription of the nucleotide sequence, and
iii) a transcription termination sequence.

16. The isolated nucleic acid molecule of claim 15, wherein the promoter comprises one or more properties selected from the group consisting of:
(a) the promoter is a liver-specific promoter;
(b) the promoter is a mouse transthyretin (mTTR) promoter;
(c) the promoter is a mTTR482 promoter; and
(d) the promoter comprises the nucleotide sequence of SEQ ID NO: 9.

17.-19. (canceled)

20. The isolated nucleic acid molecule of claim 15, further comprising an enhancer element.

21. The isolated nucleic acid molecule of claim 20, wherein the enhancer element comprises one or more properties selected from the group consisting of:
(a) the enhancer element is a mTTR enhancer element;
(b) the enhancer element is a mTTR enhancer element comprises the nucleotide sequence of SEQ ID NO: 8;
(c) the enhancer element is an synthetic enhancer sequence; and
(d) the synthetic enhancer sequence comprises the nucleotide sequence of SEQ ID NO: 7.

22.-24. (canceled)

25. The isolated nucleic acid molecule of claim 15, further comprising:
(a) a polypurine track (PPT), optionally wherein the PPT comprises the nucleotide sequence of SEQ ID NO: 6;
(b) a human CMV promoter region sequence, optionally wherein the CMV promoter region sequence comprises the nucleotide sequence of SEQ ID NO: 1;
(c) a 5′ or 3′ long terminal repeat (LTR) sequence;
(d) a stem loop 4 sequence, optionally wherein the stem loop 4 sequence comprises the nucleotide sequence of SEQ ID NO: 4;
(e) a primer binding site for SL123, optionally wherein the primer binding site for SL 123 comprises the nucleotide sequence of SEQ ID NO: 3; and/or
(f) a primer binding site for RU5 region, optionally wherein the RU5 region sequence comprises the nucleotide sequence of SEQ ID NO: 2.

26.-36. (canceled)

37. The isolated nucleic acid molecule of claim 15, comprising a genetic cassette expressing a Factor VIII (FVIII) polypeptide, wherein the genetic cassette comprises, from 5′ to 3′:
(a) a 5′ long terminal repeat (LTR) sequence;
(b) a liver-specific modified mouse transthyretin (mTTR) promoter comprising the nucleotide sequence of SEQ ID NO: 9;
(c) a nucleotide sequence encoding a FVIII protein comprising a nucleic acid sequence having at least 85% sequence identity to SEQ ID NO: 11 or SEQ ID NO: 14, and
(d) a 3′ LTR sequence.

38. A vector comprising the isolated nucleic acid molecule of claim 1.

39. A host cell comprising the isolated nucleic acid molecule of claim 1 or a vector comprising the isolated nucleic acid molecule of claim 1.

40. A polypeptide produced by the host cell of claim 39.

41. (canceled)

42. A pharmaceutical composition comprising the isolated nucleic acid molecule of claim 1.

43. A pharmaceutical composition comprising the vector of claim 38 and a pharmaceutically acceptable excipient.

44. (canceled)

45. A method of increasing expression of a polypeptide with FVIII activity in a subject comprising administering a nucleic acid molecule comprising a nucleotide sequence having at least 80% sequence identity to SEQ ID NO: 11, SEQ ID NO: 14, or SEQ ID NO: 16.

46.-47. (canceled)

48. The method of claim 45, wherein the bleeding disorder is hemophilia A.
